(19) United States
(12) Patent Application Publication
(10) Pub. No.: US 2023/0095192 A1
(43) Pub. Date: Mar. 30, 2023

(54) SYNTHETIC AGONISTS OF DNA-PK AND THEIR USE

(71) Applicant: University of Washington, Seattle, WA (US)

(72) Inventors: Daniel Brewster STETSON, Seattle, WA (US); Katelyn BURLEIGH, Seattle, WA (US); Emily SCHUTSKY, Seattle, WA (US)

(21) Appl. No.: 17/759,210

(22) PCT Filed: Mar. 1, 2021
(86) PCT No.: PCT/US2021/020254

§ 371 Date: Jul. 21, 2022

Related U.S. Application Data
(60) Provisional application No. 62/984,462, filed on Mar. 3, 2020.

(51) Int. Cl. C12N 15/117; A61P 37/02

(52) U.S. Cl. CPC C12N 15/117; A61P 37/02; C12N 2310/17; C12N 2310/315; C12N 2310/332; C12N 2310/52

(57) ABSTRACT

Polynucleotide constructs are disclosed, that are potent activators of a STING-independent DNA sensing pathway (SIDSP), in which the DNA damage response protein DNA-PK is the sensor of the SIDSP, along with tire heat shock protein HSPA8 as a unique SIDSP target. The polynucleotide constructs of the disclosure are also potent activators of the cGAS-STING DNA sensing pathway. These pathways arm key components of the innate immune response that is important for antiviral immunity, contributes to specific autoimmune diseases, and mediate important aspects of antitumor immunity.

### CROSS REFERENCE

This application claims priority to U.S. Provisional Patent Ser. No. 62/984,462 filed Mar. 3, 2020, incorporated by reference herein in its entirety.

### FEDERAL FUNDING STATEMENT

This invention was made with government support under Grant No. R21 AI130940, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Mar. 1, 2021, having the file name “19-335-PCT_Sequence-Listing_ST25.txt” and is 55 kb in size.

### BACKGROUND

Detection of intracellular DNA by innate immune sensors activates potent antiviral and inflammatory responses. The DNA sensor cGAS and its signaling adaptor STING comprise one important pathway that triggers DNA-activated innate immunity. We recently discovered that human cells have a second, potent, DNA-activated antiviral pathway that is independent of cGAS-STING. The sensor of this STING-independent DNA sensing pathway (SIDSP) is the DNA-dependent protein kinase (DNA-PK). This raises the possibility of designing synthetic agonists that trigger the SIDSP and stimulate potent responses. We describe the unique features of these agonists in this application.

### SUMMARY

In one aspect, the disclosure provides polynucleotide constructs, comprising:

(a) a first polynucleotide at least 35 nucleotides in length, wherein the first polynucleotide has a 5′ end and a 3′ end;

(b) a second polynucleotide at least 35 nucleotides in length, wherein the first polynucleotide has a 5′ end and a 3′ end;

wherein the first polynucleotide and the second polynucleotide are base-paired to form a double-stranded region of the polynucleotide construct, wherein the double-stranded region is at least 25 contiguous base pairs in length. In one embodiment, one or more of the first polynucleotide, and the second polynucleotide comprise a single stranded region at the 5′ end and/or the 3′ end.

In another aspect, the disclosure provides polynucleotides comprising a nucleic acid sequence at least 80%, 85%, 90%, 91%, 92%, 93%, 94%, 95%, 96%, 97%, 98%, 99%, or 100% identical to the nucleic acid sequence selected from the group consisting of SEQ ID NOS:1-30, 33-36, 41-42, 44-45, 51, 53, 55, 59, 61, 81, 83, 85, 87, 89, 92, 105-108, 125-132, 135-140, 145-147, 149, 151, 154-165, 168-169, 174-181, 184-185, 190-191, 196-199, 206-207, 214-217, 220-221, and 224-22S, wherein (a) underlined residues are unpaired overhangs, (b) asterisks between bases denote individual phosphorothioate (PT) linkages, and (c) abasic residues are noted in brackets.

In a further aspect, the disclosure provides methods for treating autoimmune disorders, infections (such as viral infections), and tumors, comprising administering to a subject in need thereof an amount effective to treat the autoimmune disorders, infections (such as viral infections), and tumors, of the polynucleotide construct or pharmaceutical composition of any embodiment or combination of embodiments herein.

In another aspect, the disclosure provides methods for stimulating an immune response, comprising administering to a subject in need thereof an amount effective to stimulate an immune response in a subject in need thereof of the polynucleotide construct or pharmaceutical composition of any embodiment or combination of embodiments herein.

In one aspect, the disclosure provides kits comprising

(a) a first polynucleotide at least 35 nucleotides in length, wherein the first polynucleotide has a 5′ end and a 3′ end;

(b) a second polynucleotide at least 35 nucleotides in length, wherein the first polynucleotide has a 5′ end and a 3′ end;

wherein the first polynucleotide and the second polynucleotide are capable of base-pairing to form a double-stranded region of at least 25 contiguous base pairs in length; and

wherein optionally one or both of the first polynucleotide and the second polynucleotide comprise a single stranded region at the 5′ end and/or the 3′ end.

### DESCRIPTION OF THE FIGURES

FIG. 1 shows that nucleotide content of DNA overhangs alters DNA-PK activation. Double-stranded substrates wee designed to contain 35 base pairs of complementary DNA and either 3 bases or 5 bases of unpaired overhangs on both ends (Y-form DNA); these overhangs were mononucleotide in nature, comprising all As, Ts, Cs, or Gs (exact sequences listed below, with unpaired overhangs underlined). Duplex DNAs were annealed and transfected into primary human fibroblasts at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize phosphorylation events.

FIG. 2 shows that DNA-PK activation is dependent upon the size of the DNA ligands. Double-stranded substrates were designed to contain 15, 20, 25, 30, or 35 base pairs of complementary DNA and 5 bases of unpaired cytosine-containing overhangs on both ends (Y-form DNA). Duplex DNAs were annealed and transfected into primary human fibroblasts at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize phosphorylation events.

FIG. 3 shows that 35 bp phosphorothioate (PT) substrates are potent activators of DNA-PK, but do not induce IFNB via the SIDSP. Double-stranded substrates were designed to contain 35 base pairs of complementary DNA and 5 bases of unpaired cytosine-containing overhangs on both ends (Y-form DNA). Substrates were also designed with either canonical phosphodiester linkages between bases, or phosphorothioate linkages (PT). A) Duplex DNAs were annealed and transfected into primary human fibroblasts at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize the phosphorylation events.

FIG. 4 shows that longer (>=50 bp) double-stranded oligonucleotide substrates stimulate IFNB production via the SIDSP. Duplex oligos were designed either as blunt (B) or Y-form (Y) substrates (with 5 bp C-containing overhangs), of length 35, 45, 50, 55, 60, 65, and 80 bp, such that the length denoted refers to the length of the complementary, double-stranded regions and excludes the single-stranded overhangs (in the case of Y-form DNA). All duplex DNAs were tested against ISD100, a 100 bp blunt dsDNA. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 5A-B show that 65 bp phosphodiester ligands stimulate IFNB production via the SIDSP, but phosphorothioate ligands of the same size do not. (A) Duplex oligos were designed as Y-form (Y) 65 bp substrates with 5 bp C-containing overhangs, containing either phosphodiester linkages or phosphorothioate (PT) linkages across the double-stranded portion of the ligand. A) Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate. (B) Duplex DNAs were annealed and transfected into tert-immortalized human foreskin fibroblasts (pretreated with either DMSO or the DNA-PK inhibitor Nu-7441 at 2 uM) at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize the phosphorylation events.

FIG. 6 shows that phosphorothioate linkages in the middle of one or both strands of 65Y ligands inhibits SIDSP activation. Duplex oligos were designed as Y-form (Y) 65 bp substrates with 5 bp C-containing overhangs, containing either phosphodiester linkages or phosphorothioate (PT) linkages as denoted by solid lines. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate.

FIG. 7 shows that highest SIDSP activation is achieved with Y-form DNA, including both 5′ and 3′ overhangs on each end. Duplex oligos were designed as 65 bp substrates with either 5′, 3′, or single-sided 5 C-containing overhangs. A) Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate.

FIG. 8A-C shows that PT linkages display drastic position dependent effects, in which “middle” PT linkages inhibit SIDSP activation (A), whereas PT linkages at the end or the single-/double-stranded “boundary” region of the substrate promotes potent SIDSP activation (B-C). Duplex oligos were designed as Y-form (Y) 65 bp substrates with 5 bp C-containing overhangs, containing either phosphodiester linkages as denoted by black dashed lines or phosphorothioate (PT) linkages as denoted by solid lines or stars. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate.

FIG. 9 further shows the independent, additive effects of PT linkages at the single-stranded/double-stranded boundary region of Y-form duplexes. Duplex oligos were designed as Y-form (Y) 65 bp substrates with 5 bp C-containing overhangs, containing either phosphodiester linkages as denoted by black dashed lines or phosphorothioate (PT) linkages as denoted by solid lines or stars. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate. This figure summarizes the result of multiple different experiments; therefore, for accurate comparisons between results from different experiments, all values were normalized to those of 65YS/65YAS (1) from their experiment and plotted on the same graph.

FIG. 10 shows that the potent SIDSP ligands from FIGS. 8 and 9 also activate cGAS/STING. Duplex DNAs were annealed and transfected into tert-immortalized human foreskin fibroblasts at 4 ug/mL final concentration using lipofectamine reagent following a 1 hour pretreatment with either DMSO (1:2500 dilution) or the DNA-PK inhibitor Nu7441 at 2 uM. After 6 hours, cells were harvested in RIPA buffer and lysates were run through gel electrophoresis. Proteins were transferred to nitrocellulose membranes, blocked with 5% BSA in TBST, washed, and blotted with antibodies to recognize phosphorylation events.

FIG. 11 shows that combining PT linkages at both the termini and single-stranded/double-stranded boundary regions does not activate the SIDSP to a greater extent than with PT linkages at the boundary regions alone. Duplex oligos were designed as Y-form (Y) 65 bp substrates with 5 bp C-containing overhangs, containing either phosphodiester linkages or phosphorothioate (PT) linkages. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 12A-B shows the effect of overhang length (A) and content (B) on the SIDSP-induced interferon response in the context of 65 base pair oligo duplex ligands. Duplex oligos were designed as Y-form 65 bp substrates with 5′ and 3′ C-containing overhangs ranging from 1-12 bp in length (A) or with 5 bp overhangs of differing nucleotide content (B). Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 13 shows the effect of increasing the number of Y branches in a single ligand molecule on SIDSP activation. A) Oligos were designed as Y-form 65 bp substrates with 5 bp C-containing overhangs. DNAs were annealed either as a duplex (2 strands), triplex (3 strands), or quadruplex (4 strands), and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 14 shows the effect of GC content in the double-stranded portion of the SIDSP agonist. Oligos were designed as Y-form 65 bp substrates with 5 bp C-containing overhangs. Starting with two distinct sequences (“65Y” and “4XO”), minimal changes were made in order to drastically increase or decrease the GC content of the double-stranded portion. DNAs were annealed as a duplex and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 15 further shows the effect of GC content in the double-stranded portion of the SIDSP agonist. Oligos were designed as Y-form 65 bp substrates with 5 bp C-containing overhangs. DNAs were annealed as a duplex and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 16 shows the effect of abasic sites in the overhangs of the prototype SIDSP agonists. Oligos were designed as Y-form 65 bp substrates with 5 bp overhangs of either deoxycytosines (C), deoxythymines (T), or deoxyuracils (U). DNAs were annealed as a duplex, and then treated either with mock buffer or with Uracil DNA glycosylase (UDG) in UDG buffer for 10 minutes at 37 C, followed by a heat quench at 95 C for 10 minutes. Uracil DNA glycosylase inhibitor (UGI) was then added in excess to completely stop any further reaction. UDG treatment of DNAs containing deoxyuracils will generate abasic sites. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 17 shows the effect of specific abasic sites in the overhangs of the prototype SIDSP agonists. Oligos were designed as Y-form 65 bp substrates with 5 bp overhangs containing a mixture of deoxycytosines (C) and deoxyuracils (U). DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 18A-B shows the effect of natural versus synthetic abasic sites on the potency of prototype SIDSP agonists. Oligos were designed as Y-form 65 bp substrates with 5 bp overhangs containing either a mixture of dcoxycytosines (C) and deoxyuracils (U)-leftmost structure in FIG. 18B, denoting 1′ OH— or a mixture of deoxycytosines (C) and “dSpacer” modifications from IDT-rightmost structure in FIG. 18B, denoting lack of 1′ OH. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 19 shows the effect of abasic sites in otherwise inert prototype SIDSP agonists. Oligos were designed as Y-form 65 bp substrates with 5 bp overhangs containing either deoxycytosines (C), deoxythymines (T), or deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 20 shows the effect of abasic sites in specific locations in the overhangs of prototype SIDSP agonists. Oligos were designed as Y-form 65 bp substrates with 5 bp overhangs containing a mixture of deoxycytosines (C) and deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 21 shows the effect of abasic sites in the overhangs of shorter prototype SIDSP agonists. Oligos were designed as Y-form substrates where the double-stranded region was either 65 bp or 40 bp. All oligos contained 5 bp overhangs with a mixture of deoxycytosines (C) and deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPD14 loci. IFNB transcript levels were normalized to those of GAPDH for each condition. Experiment was performed in biological triplicate and technical duplicate.

FIG. 22 shows the effect of abasic sites at specific positions in the overhangs of prototype SIDSP agonists. Oligos were designed as 65 bp Y-form substrates containing 5 bp overhangs with a mixture of deoxycytosines (C) and deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 23 shows the effect of combining phosphorothioate linkages and abasic sites at specific positions in the overhangs of prototype SIDSP agonists. Oligos were designed as 65 bp Y-form substrates containing 5 bp overhangs with a mixture of deoxycytosines (C) and deoxyuracils (U) as denoted in the sequences below. Asterisks denote the locations of phosphorothioate linkages. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Dircectol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

FIG. 24A-C provides a cartoon representation of the (A) two-, (B) three-, and (C) four-prong embodiments of constructs of the disclosure.

FIG. 25 shows an exemplary chemical representation of the ring opening form of abasic sites.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutsheer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein. “about” means+/−5% of the recited parameter.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polynucleotide constructs, comprising:

(a) a first polynucleotide at least 35 nucleotides in length, wherein the first polynucleotide has a 5′ end and a 3′ end;

(b) a second polynucleotide at least 35 nucleotides in length, wherein the first polynucleotide has a 5′ end and a 3′ end;

wherein the first polynucleotide and the second polynucleotide are base-paired to form a double-stranded region of the polynucleotide construct, wherein the double-stranded region is at least 25 contiguous base pairs in length.

In one embodiment, one or both of the first polynucleotide and the second polynucleotide comprise a single stranded region at the 5′ end and/or the 3′ end.

As disclosed herein, the inventors have discovered that the polynucleotide constructs of the disclosure are potent activators of a STING-independent DNA sensing pathway (SIDSP) they recently identified, in which the DNA damage response protein DNA-PK is the sensor of the SIDSP, along with the heat shock protein HSPA8 as a unique SIDSP target. The polynucleotide constructs of the disclosure are also potent activators of the cGAS-STING DNA sensing pathway. These pathways are key components of the innate immune response that is important for antiviral immunity, contributes to specific autoimmune diseases, and mediates important aspects of antitumor immunity. Thus, the polynucleotide constructs of the disclosure can be used, for example, to treat autoimmune disorders, infections (such as viral infections), and tumors.

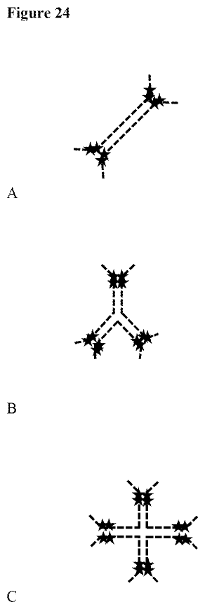

In one embodiment, the polynucleotide construct comprises the first and second polynucleotides (two prong embodiment). In other embodiments, the construct may comprise a third polynucleotide (three-prong embodiment) or a third polynucleotide and a fourth polynucleotide (four prong embodiment). FIG. 24 provides a cartoon representation of the two-, three-, and four-prong embodiments.

In the three prong embodiment, the construct further comprises:

(c) a third polynucleotide at least 35 nucleotides in length, wherein the third polynucleotide has a 5′ end and a 3′ end; wherein
(i) the first polynucleotide and the third polynucleotide are base-paired to form a second double-stranded region of the polynucleotide construct, wherein the second double-stranded region is at least 25 contiguous base pairs in length; and
(ii) the second polynucleotide and the third polynucleotide are base-paired to form a third double-stranded region of the polynucleotide construct, wherein the second double-stranded region is at least 25 contiguous base pairs in length.

In one embodiment, the third polynucleotide comprises a single stranded region at the 5′ end and/or the 3′ end.

In the four prong embodiment, the construct further comprises

(c) a third polynucleotide at least 35 nucleotides in length, wherein the third polynucleotide has a 5′ end and a 3′ end; and

(d) a fourth polynucleotide at least 35 nucleotides in length, wherein the fourth polynucleotide has a 5′ end and a 3′ end, wherein
(i) the first polynucleotide and the third polynucleotide are base-paired to form a second double-stranded region of the polynucleotide construct, wherein the second double-stranded region is at least 25 contiguous base pairs in length;
(ii) the second polynucleotide and the fourth polynucleotide are base-paired to form a third double-stranded region of the polynucleotide construct, wherein the third double-stranded region is at least 25 contiguous base pairs in length; and
(iii) the third polynucleotide and the fourth polynucleotide are base-paired to form a fourth double-stranded region of the polynucleotide construct, wherein the fourth double-stranded region is at least 25 contiguous base pairs in length.

In one embodiment, the third polynucleotide and/or the fourth polynucleotide may comprise a single stranded region at the 5′ end and/or the 3′ end.

The polynucleotides may comprise any residues capable of base-pairing as recited, including but not limited to deoxyadenosine (A), deoxythymidine (T), deoxycytosine (C), deoxyguanine (G), and deoxyuracil (U). Polynucleotides are each at least 35 nucleotides in length and can be of any suitable length. In one non-limiting embodiment, the first and second polynucleotides are polynucleotides and are independently between 35-250 nucleotides in length. The polynucleotides may be the same length, or may differ in length. In a specific embodiment, the polynucleotides are the same length.

The polynucleotides are base-paired to form one or more double-stranded regions of the polynucleotide construct, wherein the double-stranded region is at least 25 contiguous base pairs in length. In one embodiment, the double-stranded regions are perfectly double-stranded (i.e. no mismatches); in another embodiment, the double-stranded regions may include 1 or 2 mismatches.

The double-stranded regions may be of any suitable length. In one embodiment, the double stranded regions are between 25 contiguous base pairs in length and 200 contiguous base pairs in length. In various further embodiments, the double stranded regions are between 25 contiguous base pairs in length and 175 contiguous base pairs in length, 25 contiguous base pairs in length and 150 contiguous base pairs in length, 25 contiguous base pairs in length and 125 contiguous base pairs in length, 25 contiguous base pairs in length and 100 contiguous base pairs in length, 25 contiguous base pairs in length and 80 contiguous base pairs in length, 35 contiguous base pairs in length and 200 contiguous base pairs in length, 35 contiguous base pairs in length and 175 contiguous base pairs in length, 35 contiguous base pairs in length and 150 contiguous base pairs in length, 35 contiguous base pairs in length and 125 contiguous base pairs in length, 35 contiguous base pairs in length and 100 contiguous base pairs in length, 35 contiguous base pairs in length and 80 contiguous base pairs in length, 45 contiguous base pairs in length and 200 contiguous base pairs in length, 45 contiguous base pairs in length and 175 contiguous base pairs in length, 45 contiguous base pairs in length and 150 contiguous base pairs in length, 45 contiguous base pairs in length and 125 contiguous base pairs in length, 45 contiguous base pairs in length and 100 contiguous base pairs in length, 45 contiguous base pairs in length and 80 contiguous base pairs in length. In specific embodiments, the double stranded regions are at least 55 contiguous base pairs, at least 60 contiguous base pairs, or at least 65 contiguous base pairs. In other specific embodiments, the double stranded regions are between 55 contiguous base pairs in length and 75 contiguous base pairs in length, between 60 contiguous base pairs in length and 75 contiguous base pairs in length, between 55 contiguous base pairs in length and 70 contiguous base pairs in length, between 60 contiguous base pairs in length and 70 contiguous base pairs in length, or about 55, 56, 57, 58, 59, 60, 61, 62, 63, 64, 65, 66, 67, 68, 69, 70, 71, 72, 73, 74, or 75 contiguous base pairs in length. In other embodiments, the double stranded region is between 30 contiguous base pairs in length and 50 contiguous base pairs in length for three prong and four prong embodiments.

The double stranded regions may comprise any sequence of residues suitable for an intended purpose. In one embodiment, the double stranded regions have a GC nucleotide content of at least 10%, 20%, 30%, 40%, 50%, or more. In another embodiment, the double stranded regions have a GC nucleotide content of 20% or less, 15% or less, 10% or less, 9% or less, 8% or less, 7% or less, 6% or less, 5% or less, 4% or less, 3% or less, 2% or less, 1% or less, or 0%. As shown in the examples that follow, constructs with a low GC content in the double stranded region exhibit particularly good SIDSP stimulatory activity.

In the polynucleotide constructs of the disclosure, one or both of the first polynucleotide and the second polynucleotide may comprise a single stranded region at the 5′ end and/or the 3′ end. In various embodiments:
The first polynucleotide has a single stranded region at its 5′ end;
The first polynucleotide has a single stranded region at its 3′ end:
The first polynucleotide has a single stranded region at its 5′ end and its 3′ end;
The second polynucleotide has a single stranded region at its 5′ end;
The second polynucleotide has a single stranded region at its 3′ end;
The second polynucleotide has a single stranded region at its 5′ end and its 3′ end;
The first polynucleotide and the second polynucleotide each have a single stranded region at their 5′ ends;
The first polynucleotide and the second polynucleotide each have a single stranded region at their 3′ ends;
The first polynucleotide has a single stranded region at its 5′ end and the second polynucleotide has a single stranded region at its 3′ end;
The first polynucleotide has a single stranded region at its 3′ end and the second polynucleotide has a single stranded region at its 5′ end; or
The first polynucleotide and the second polynucleotide each have a single stranded region at their 5′ ends and a single stranded region at their 3′ ends.

Similarly, the third and fourth polynucleotides, when present, may comprise a single stranded region at their 5′ ends and/or their 3′ ends.

In a specific embodiment, both the first polynucleotide and the second polynucleotide comprise a single stranded region at each of the 5′ end and the 3′ end, and wherein, when present, both the third polynucleotide and the fourth polynucleotide comprise a single stranded region at each of the 5′ end and the 3′ end.

Any suitable length of single stranded region may be present in the polynucleotide constructs. In various embodiments, each single stranded region may independently be 1, 2, 3, 4, 5, 6, 7, 8, 9, 10, 11, or 12 nucleotides. In various embodiments, each single stranded region may be between 3-12, 3-11, 3-10, 3-9, 3-8, 3-7, 3-6, 3-5, 3-4, 4-12, 4-11, 4-10, 4-9, 4-8, 4-7, 4-6, 4-5, 5-12, 5-11, 5-10, 5-9, 5-8, 5-7, 5-6, 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 nucleotides in length. Each single stranded region may be the same length, or the lengths of different single stranded regions may differ. In one embodiment, each single stranded region is the same length. In one specific embodiment, each single stranded region is between 3 nucleotides and 7 nucleotides in length, or 3, 4, 5, 6, or 7 nucleotides in length. In another specific embodiment, each single stranded region is between 3 nucleotides and 5 nucleotides in length, or 3, 4, or 5 nucleotides in length.

Any suitable nucleotide composition of the single stranded regions may be present in the polynucleotide constructs. In one embodiment, each single stranded region comprises at least one pyrimidine. In another embodiment, the single stranded regions include 1, 2, 3, 4, 5, 6, 7, 8, 9, 10, 11, or 12 pyrimidines. In a further embodiment, each single stranded region comprises at least one cytosine. In another embodiment, each single stranded region includes 1, 2, 3, 4, 5, 6, 7, 8, 9, 10, 11, or 12 deoxycytosines.

In one specific embodiment, each single stranded region comprises all pyrimidines. In another specific embodiment, each single stranded region comprises all deoxycytosines.

In another embodiment, one or more of the single stranded regions one or more deoxyuracil residues. As described in the examples that follow, such deoxyuracil-containing polynucleotides can be treated with, for example, uracil DNA glycosylase (UDG), to produce abasic sites where deoxyuracil residues had been present. Thus, such deoxyuracil-containing polynucleotides may be used, for example, as intermediates to generate polynucleotides containing one or more abasic sites. Such abasic-site containing polynucleotides are shown in the examples as potent activators of a SIDSP. In one embodiment, 2, 3, 4, or more of each single stranded region comprise one or more deoxyuracil residue, or each single stranded region comprise one or more deoxyuracil residue. In another embodiment, the deoxyuracil residues are present at the residue in the single stranded region that abuts the double stranded region (also referred to herein as the “inner residue” of the single stranded region). In a further embodiment, a deoxyuracil residue is present in the middle of 1, 2, 3, 4, or more of the single stranded regions.

In further specific embodiments, 1, 2, 3, 4, or more of each single stranded region comprise one or more abasic site, or each single stranded region comprise one or more abasic site. As used herein, an “abasic site” is a residue in which the DNA sugar-phosphate backbone is intact, but the sugar is not linked to a DNA base. Specifically, the N-glycosidic bond between the 1′ position of the sugar and the base is cleaved, releasing the base and leaving an abasic site. The resulting sugar can exist in a furanose ring conformation or can undergo a ring-opening reaction to exist in an open-chain free aldehyde or free alcohol conformation. In one embodiment, the abasic site is generated by uracil DNA glycosylase. However, any suitable procedure for generating abasic sites may be used, including but not limited to those disclosed in published PCT application WO 2016/164363 and U.S. Pat. No. 6,586,586, each incorporated by reference herein in its entirety. Non-limiting examples of such procedures are detailed in Table 1.

TABLE 1
Adapted from WO2016164363A1 and Jacobs AL, et al. (2012) “DNA
glycosylases: in DNA repair and beyond.” Chromosoma 121(1): 1-20.

| Glycosylase | Substrates |
|---|---|
| Uracil DNA glycosylase (UDG) | Uracil, 5-fluorouracil, isodiauric acid, |
| | 5-hydroxyuracil, alloxan |
| Thymine DNA glycosylase (TDG) | 5-formylcytosine (5fC), 5-carboxylcytosine |
| | (5caC) |
| G/T(U) mismatch DNA glycosylase | G/G, A/G, T/C, T/U, and U/C mismatches, |
| (e.g. MUG, MBD4) | uracil mismatch |
| Alkylbase-DNA glycosylases | 3-methyl guanine, O2-alkylcytosine, |
| (e.g. MPG for 3-methyladenine; | 5-formyluracil, 5-hydroxymethyluracil, |
| OGG1 for 8-oxoguanine) | hypoxanthine, N6-ethenoadenine, |
| | N4-ethenocytosine, 7-chloroethyl-guanine, |
| | 3-methyladenine, 8-oxoguanine |
| 5-methylcytosine-DNA glycosylase | T in G/T mismatch, 5-methylcytosine |
| (e.g. DEMETER) | |
| Single-strand selective monofunctional | Uracil, 5-hydroxyuracil, |
| uracil-DNA glycosylase (SMUG1) | 5-hydroxymethyluracil, 5-formyluracil |

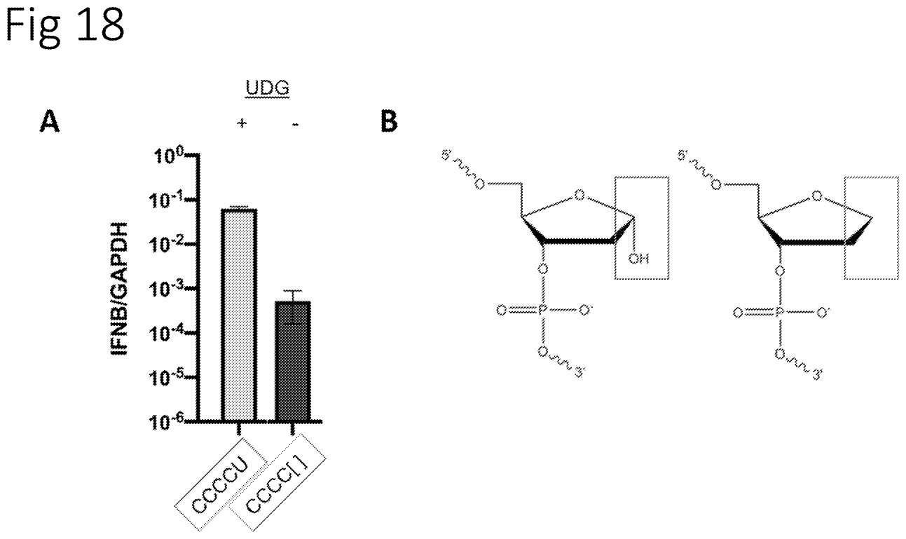

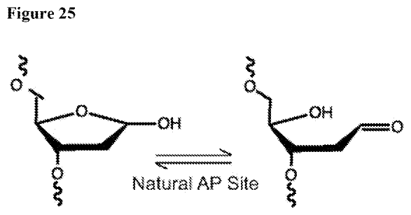

In one embodiment the abasic site has the structure shown in FIG. 18B, left panel. In a further embodiment, the abasic site has the structure shown in FIG. 25, right panel. FIG. 25 shows an exemplary chemical representation of the ring opening form of the abasic sites (Wang, W et al. (2018) PNAS 115(11): E2538-E2545. DOI: 10.1073/pnas.1722050115)

In another embodiment, the one or more abasic sites are present at the residue in the single stranded region that abuts the double stranded region (also referred to herein as the “inner residue” of the single stranded region). In another embodiment, an abasic site is present in the middle of 1, 2, 3, 4, or more of the single stranded regions. As described in the examples that follow, abasic residue-containing constructs in which the abasic residues are present at the single stranded/double stranded boundary or in the middle of the single stranded regions provide the greatest effect on SIDSP activity.

The polynucleotide constructs may include phosphorothioate linkages between nucleotides at one or more boundaries of the single stranded region and the double stranded region. The “boundaries” are the linkage between the terminal residue in the double-stranded region, and the first residue in the single stranded region of the polynucleotides. Thus, there are four such boundaries in the two prong embodiment, six such boundaries in the three prong embodiment, and eight such boundaries in the four prong embodiment, in various embodiments, the polynucleotide constructs include phosphorothioate linkages between nucleotides at 1, 2, 3, 4, 5, 6, 7, or all 8 boundaries of the single stranded region and the double stranded region. In other embodiments, the polynucleotide constructs may include phosphorothioate linkages between nucleotides in 1, 2, 3, 4, 5, 6, 7, or all 8 of the single stranded regions. In one specific embodiment, the construct comprises one or more phosphorothioate linkages between nucleotides at all boundaries of the single stranded region and the double stranded region. In a further specific embodiment, the construct comprises one or more phosphorothioate linkages between nucleotides within one or more of the single stranded regions. In another specific embodiment, the polynucleotide constructs do not include phosphorothioate-linkages between residues that are each in the double stranded regions. In another specific embodiment, the construct does not include phosphorothioate-linkages between nucleotides in the double-stranded regions other than at the one or more boundaries of the single stranded region and the double stranded region.

In one specific embodiment of the polynucleotide constructs,

(i) the first polynucleotide and the second polynucleotide are of equal length and are base-paired to form a perfectly double stranded region of between 55 contiguous base pairs in length and 75 contiguous base pairs, or between 55 contiguous base pairs in length and 70 contiguous base pairs, or between 60 contiguous base pairs in length and 75 contiguous base pairs, or between 60 contiguous base pairs in length and 70 contiguous base pairs, or 55, 56, 57, 58, 59, 60, 61, 62, 63, 64, 65, 66, 67, 68, 69, 70, 71, 72, 73, 74, or 75 contiguous base pairs;

(ii) GC content in the double stranded region is 20% or less, 15% or less, 10% or less, 9% or less, 8% or less, 7% or less, 6% or less, 5% or less, 4% or less, 3% or less, 2% or less, 1% or less, or 0%;

(iii) the first polynucleotide comprises a single stranded region at the 5′ end and the 3′ end, wherein each single stranded region is between 3 nucleotides and 7 nucleotides in length or between 3 nucleotides and 5 nucleotides in length;

(iv) the second polynucleotide comprises a single stranded region at the 5′ end and the 3′ end, wherein each single stranded region is between 3 nucleotides and 7 nucleotides in length or between 3 nucleotides and 5 nucleotides in length;

(v) there are no phosphorothioate-linkages between residues that are each in the double stranded regions;

(v) wherein one or more of the following is true:
(A) each single stranded region comprises all pyrimidines, preferably comprising all deoxycytosines, and there is a phosphorothioate linkages between nucleotides at all boundaries of the single stranded region and the double stranded region;
(B) each single stranded region comprises one or more abasic site, wherein (i) abasic sites are present at the residue in each single stranded region that abuts the double stranded region, and/or (ii) an abasic site is present in the middle of each of the single stranded regions.

In various further embodiments, the polynucleotide constructs comprise a first polynucleotide and a second polynucleotide comprising nucleic acid sequences at least 80%, 85%, 90%, 91%, 92%, 93%, 94%, 95%, 96%, 97%, 98%, 99%, or 100% identical to the nucleic acid sequence selected from the following combinations, wherein (a) underlined residues are unpaired overhangs, (b) asterisks between bases denote individual phosphorothioate (PT) linkages, and (c) abasic residues are noted in brackets:
SEQ ID NO:1-2:
SEQ ID NO:3-4;
SEQ ID NO:5-6;
SEQ ID NO:7-8;
SEQ ID NO:9-10
SEQ ID NO: 11-12;
SEQ ID NO: 13-14;
SEQ ID NO:15-16;
SEQ ID NO:17-18;
SEQ ID NO: 19-20;
SEQ ID NO:21-22;
SEQ ID NO:23-24
SEQ ID NO:25-26;
SEQ ID NO:27-28;
SEQ ID NO:29-30;
SEQ ID NO:35-36;
SEQ ID NO: 35 and SEQ ID NO:32;
SEQ ID NO:41-42;
SEQ ID NO: 41 and SEQ ID NO:44;
SEQ ID NO: 46 and SEQ ID NO:42;
SEQ ID NO: 46 and SEQ ID NO:44;
SEQ ID NO:51 and SEQ ID NO:22:
SEQ ID NO:53 and SEQ ID NO:22;
SEQ ID NO:55 and SEQ ID NO:22;
SEQ ID NO:59 and SEQ ID NO:22;
SEQ ID NO:61 and SEQ ID NO:22;
SEQ ID NO: 59 and SEQ ID NO:36;
SEQ ID NO: 61 and SEQ ID NO:36;
SEQ ID NO: 53 and SEQ ID NO:36;
SEQ ID NO: 55 and SEQ ID NO:36;
SEQ ID NO:81 and SEQ ID NO:22;
SEQ ID NO:83 and SEQ ID NO:22;
SEQ ID NO:85 and SEQ ID NO:22;
SEQ ID NO:87 and SEQ ID NO:22;
SEQ ID NO:89 and SEQ ID NO:22;
SEQ ID NO:51 and SEQ ID NO:92;
SEQ ID NO: 81 and SEQ ID NO:92;
SEQ ID NO: 83 and SEQ ID NO:92;
SEQ ID NO: 85 and SEQ ID NO:92;
SEQ ID NO: 87 and SEQ ID NO:92;
SEQ ID NO: 89 and SEQ ID NO:92:
SEQ ID NO: 55 and SEQ ID NO:92;
SEQ ID NO:105-106;
SEQ ID NO:107-108;
SEQ ID NO:125-126;
SEQ ID NO:127-128
SEQ ID NO:129-130;
SEQ ID NO: 131-132;
SEQ ID NO: 135-136;
SEQ ID NO:137-138;
SEQ ID NO:139-140:
SEQ ID NO:154-155;
SEQ ID NO:156-157;
SEQ ID NO:160-161;
SEQ ID NO:162-163;
SEQ ID NO:164-165;
SEQ ID NO:168-169;
SEQ ID NO:174-175;
SEQ ID NO: 176-177;
SEQ ID NO: 178-179;
SEQ ID NO:180-181;
SEQ ID NO:184-185;
SEQ ID NO:190-191;
SEQ ID NO:196-197:
SEQ ID NO:198-199:
SEQ ID NO:206-207;
SEQ ID NO:214-215;
SEQ ID NO:216-217;
SEQ ID NO:220-221;
SEQ ID NO:224-225;
SEQ ID NO:145-147 (Triplex); and
SEQ ID NO: 145, 146, 149, and 151 (Quadruplex).

In other embodiments, the polynucleotide constructs comprise a first polynucleotide and a second polynucleotide comprising a nucleic acid sequence at least 80%, 85%, 90%, 91%, 92%, 93%, 94%, 95%, 96%, 97%, 98%, 99%, or 100% identical to the nucleic acid sequence selected from the following combinations, wherein (a) underlined residues are unpaired overhangs, (b) asterisks between bases denote individual phosphorothioate (PT) linkages, and (c) abasic residues are noted in brackets:
SEQ ID NO:3-4;
SEQ ID NO:7-8;
SEQ ID NO: 19-20;
SEQ ID NO:21-22;
SEQ ID NO:25-26;
SEQ ID NO:27-28;
SEQ ID NO:41-42;
SEQ ID NO: 41 and SEQ ID NO:44;
SEQ ID NO: 46 and SEQ ID NO:42;
SEQ ID NO: 46 and SEQ ID NO:44;
SEQ ID NO:53 and SEQ ID NO:22;
SEQ ID NO:55 and SEQ ID NO:22;
SEQ ID NO:59 and SEQ ID NO:22;
SEQ ID NO:61 and SEQ ID NO:22;
SEQ ID NO: 59 and SEQ ID NO:36;
SEQ ID NO: 61 and SEQ ID NO:36;
SEQ ID NO: 53 and SEQ ID NO:36;
SEQ ID NO: 55 and SEQ ID NO:36;
SEQ ID NO:83 and SEQ ID NO:22;
SEQ ID NO:85 and SEQ ID NO:22;
SEQ ID NO:87 and SEQ ID NO:22;
SEQ ID NO:89 and SEQ ID NO:22;
SEQ ID NO: 51 and SEQ ID NO:92;
SEQ ID NO: 81 and SEQ ID NO:92;
SEQ ID NO: 83 and SEQ ID NO:92;
SEQ ID NO: 85 and SEQ ID NO:92;
SEQ ID NO: 87 and SEQ ID NO:92;
SEQ ID NO: 89 and SEQ ID NO:92;
SEQ ID NO: 55 and SEQ ID NO:92;
SEQ ID NO:105-106;
SEQ ID NO:107-108;
SEQ ID NO:125-126;
SEQ ID NO:129-130;
SEQ ID NO:131-132;
SEQ ID NO:135-136;
SEQ ID NO:137-138;
SEQ ID NO: 156-157;
SEQ ID NO:160-161;
SEQ ID NO:162-163;
SEQ ID NO:164-165;
SEQ ID NO:168-169;
SEQ ID NO:174-175;
SEQ ID NO:176-177;
SEQ ID NO: 178-179;
SEQ ID NO:180-181;
SEQ ID NO:190-191;
SEQ ID NO:196-197;
SEQ ID NO: 198-199;
SEQ ID NO:214-215;
SEQ ID NO:216-217;
SEQ ID NO:220-221;
SEQ ID NO:224-225;
SEQ ID NO:145-147 (Triplex); and
SEQ ID NO: 145, 146, 149, and 151 (Quadruplex).

In further embodiments, the polynucleotide constructs comprise a first polynucleotide and a second polynucleotide comprising a nucleic acid sequence at least 80%, 85%, 90%, 91%, 92%, 93%, 94%, 95%, 96%, 97%, 98%, 99%, or 100% identical to the nucleic acid sequence selected from the following pairs, wherein (a) underlined residues are unpaired overhangs, (b) asterisks between bases denote individual phosphorothioate (PT) linkages, and (c) abasic residues are noted in brackets:
SEQ ID NO:53 and SEQ ID NO:22;
SEQ ID NO:55 and SEQ ID NO:22;
SEQ ID NO: 55 and SEQ ID NO:36;
SEQ ID NO: 83 and SEQ ID NO:92;
SEQ ID NO: 89 and SEQ ID NO:92;
SEQ ID NO: 55 and SEQ ID NO:92;
SEQ ID NO:105-106;
SEQ ID NO:107-108;
SEQ ID NO:125-126;
SEQ ID NO:160-161;
SEQ ID NO:162-163;
SEQ ID NO:168-169;
SEQ ID NO:180-181;
SEQ ID NO:190-191;
SEQ ID NO:216-217;
SEQ ID NO:220-221; and
SEQ ID NO:224-225.

In another embodiment, the disclosure provides individual polynucleotides of the disclosure (i.e.: first, second, third, or fourth polynucleotides), which can be used, for example, to prepare the polynucleotide constructs of the present disclosure. In one embodiment, the polynucleotides comprise a nucleic acid sequence at least 80%, 85%, 90%, 91%, 92%, 93%, 94%, 95%, 96%, 97%, 98%, 99%, or 100% identical to the nucleic acid sequence selected from the group consisting of SEQ ID NOS:1-30, 33-36, 41-42, 44-45, 51, 53, 55, 59, 61, 81, 83, 85, 87, 89, 92, 105-108, 125-132, 135-140, 145-147, 149, 151, 154-165, 168-169, 174-181, 184-185, 190-191, 196-199, 206-207, 214-217, 220-221, and 224-225, wherein (a) underlined residues are unpaired overhangs, (b) asterisks between bases denote individual phosphorothioate (PT) linkages, and (c) abasic residues are noted in brackets.

In a further aspect, the disclosure provides expression vectors comprising the polynucleotides of the disclosure operatively linked to a suitable control sequence. “Expression vector” includes vectors that operatively link a nucleic acid coding region or gene to any control sequences capable of effecting expression of the polynucleotides of the disclosure. “Control sequences” operably linked to the polynucleotides of the disclosure are nucleic acid sequences capable of effecting the expression of the polynucleoides. The control sequences need not be contiguous with the polynuclcotides, so long as they function to direct the expression thereof. Thus, for example, intervening untranslated yet transcribed sequences can be present between a promoter sequence and the polynucleotides and the promoter sequence can still be considered “operably linked” to the polynucleotides. Other such control sequences include, but are not limited to, polyadenylation signals, termination signals, etc. Such expression vectors can be of any type, including but not limited plasmid and viral-based expression vectors. The control sequence used to drive expression of the disclosed polynucleotides in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). In various embodiments, the expression vector may comprise a plasmid, viral-based vector, or any other suitable expression vector.

In another aspect, the disclosure provides host cells that comprise the polynucleotides or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another embodiment, the disclosure provides pharmaceutical composition comprising the polynucleotide constructs, polynucleotides, kits, expression vectors, and/or cells of any embodiment or combination of embodiments herein.

The pharmaceutical compositions of the disclosure can be used, for example, in the methods of the disclosure described below. The pharmaceutical composition may comprise in addition to the polynucleotide construct of the disclosure (a) a lyoprotectant; (b) a surfactant; (c) a bulking agent; (d) a tonicity adjusting agent; (c) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polynucleotide construct may be the sole active agent in the pharmaceutical composition, or the composition may further comprise one or more other active agents suitable for an intended use.

The polynucleotide construct may be used for any suitable purpose, as described in detail herein. In various non-limiting embodiments, the purpose may include treating autoimmune disorders, infections (such as viral infections), and tumors.

In another aspect, the disclosure provides methods for treating autoimmune disorders, infections (such as bacterial or viral infections), and tumors, comprising administering to a subject in need thereof an amount effective to treat the autoimmune disorders, infections (such as viral infections), and tumors, of the polynucleotide construct or pharmaceutical composition of any embodiment or combination of embodiments of the disclosure.

In another aspect, the disclosure provides methods for treating autoimmune disorders, infections (such as bacterial or viral infections), and tumors, comprising administering to a subject in need thereof an amount effective to stimulate an immune response in a subject in need thereof of the polynucleotide construct or pharmaceutical composition of any embodiment or combination of embodiments of the disclosure. In this embodiment, the polynucleotide construct or pharmaceutical composition may be used, for example, as an adjuvant to stimulate an immune response in a subject in need thereof, such as a subject in need of treatment for autoimmune disorders, infections (such as bacterial or viral infections), and tumors. Such methods may further comprise treating the subject with one or more additional therapeutics as appropriate for the subject, including but not limited to other antiviral therapeutics, anti-tumor therapeutics (including but not limited to checkpoint inhibitors such as PD1, PD-L1, CTLA4 inhibitors, etc.)

As used herein, “treat” or “treating” means accomplishing one or more of the following: (a) reducing the severity of the disease; (b) limiting or preventing development of symptoms, including flares, characteristic of the disease; (c) inhibiting worsening of symptoms characteristic of the disease; (d) limiting or preventing recurrence of the disease or symptoms in subjects that were previously symptomatic for.

The amount of therapeutics(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular therapeutics(s), etc. Dosage amounts will typically be in the range of from about 0.001 mg/kg/day, 0.001 mg/kg/day or 0.01 mg/kg/day to about 100 mg/kg/day, but may be higher or lower, depending upon, among other factors, the activity of the active therapeutic, the bioavailability of the therapeutic, its metabolism kinetics and other pharmacokinetic properties, the mode of administration and various other factors. Dosage amount and interval may be adjusted individually to provide plasma levels of the therapeutic(s) and/or active metabolite compound(s) which are sufficient to maintain therapeutic or prophylactic effect. For example, the therapeutics may be administered once per week, several times per week (e.g. every other day), once per day or multiple times per day, depending upon, among other things, the mode of administration, the specific indication being treated and the judgment of the prescribing medical personnel.

In another aspect, the disclosure provides kits comprising (a) a first polynucleotide of any embodiment the disclosure, and (b) a second polynucleotide of any embodiment of the disclosure. In this embodiment, the first and second polynucleotides may be base paired or not base paired. Such kits may optionally comprise (c) a third polynucleotide of any embodiment of the disclosure, and (d) a fourth polynucleotide of any embodiment of the disclosure. In one such embodiment, the kits comprise

(a) a first polynucleotide at least 35 nucleotides in length, wherein the first polynucleotide has a 5′ end and a 3′ end;

(b) a second polynucleotide at least 35 nucleotides in length, wherein the first polynucleotide has a 5′ end and a 3′ end;

wherein the first polynucleotide and the second polynucleotide are capable of base-pairing to form a double-stranded region of at least 25 contiguous base pairs in length; and

wherein optionally one or both of the first polynucleotide and the second polynucleotide comprise a single stranded region at the 5′ end and/or the 3′ end; and

(c) optionally a third polynucleotide at least 35 nucleotides in length, wherein the third polynucleotide has a 5′ end and a 3′ end; wherein
(i) the first polynucleotide and the third polynucleotide are capable of base pairing to form a second double-stranded region of at least 25 contiguous base pairs in length; and
(ii) the second polynucleotide and the third polynucleotide are capable of base pairing to form a third double-stranded region at least 25 contiguous base pairs in length;

wherein optionally the third polynucleotide may comprise a single stranded region at the 5′ end and/or the 3′; or

(d) optionally, (i) a third polynucleotide at least 35 nucleotides in length, wherein the third polynucleotide has a 5′ end and a 3′ end; and (ii) a fourth polynucleotide at least 35 nucleotides in length, wherein the fourth polynucleotide has a 5′ end and a 3′ end, wherein
(A) the first polynucleotide and the third polynucleotide are capable of base pairing to form a second double-stranded region at least 25 contiguous base pairs in length;
(B) the second polynucleotide and the fourth polynucleotide are capable of base pairing to form a third double-stranded region at least 25 contiguous base pairs in length; and
(C) the third polynucleotide and the fourth polynucleotide are capable of base pairing to form a fourth double-stranded region of at least 25 contiguous base pairs in length

wherein the third polynucleotide and/or the fourth polynucleotide may optionally comprise a single stranded region at the 5′ end and/or the 3′ end.

All embodiments of the polynucleotide constructs disclosed above are suitable for use in the kits of the disclosure. As will be understood by those of skill in the art, the first and second polynucleotides in the kit may each be single stranded (i.e.: not base-paired), which can then be combined under appropriate condition to promote base pairing and formation of constructs of the disclosure prior to an intended use. In these embodiments, reference is made to double-stranded regions “when formed” or “capable of being formed”, and will be understood to refer to those portions of the polynucleotides that will base pair to form the constructs of the disclosure. Thus, for example, in one embodiment each double-stranded region when formed is perfectly double-stranded. In another embodiment, each double stranded region when formed is between 25 contiguous base pairs in length and 200 contiguous base pairs in length. In further embodiments, the double stranded region when formed is between 35 contiguous base pairs in length and 110 contiguous base pairs in length, or between 45 contiguous base pairs in length and 80 contiguous base pairs in length for two prong embodiments, or between 30 contiguous base pairs in length and 50 contiguous base pairs in length for three prong and 4 prong embodiments. In one embodiment, the double stranded region when formed is between 45 contiguous base pairs in length and 110 contiguous base pairs in length, or between 45 contiguous base pairs in length and 80 contiguous base pairs in length for two prong embodiments.

In one embodiment, both the first polynucleotide and the second polynucleotide comprise a single stranded region at the 5′ end and/or the 3′ end, and wherein, when present, both the third polynucleotide and the fourth polynucleotide comprise a single stranded region at the 5′ end and/or the 3′ end. In another embodiment, both the first polynucleotide and the second polynucleotide comprise a single stranded region at each of the 5′ end and the 3′ end, and wherein, when present, both the third polynucleotide and the fourth polynucleotide comprise a single stranded region at each of the 5′ end and the 3′ end. In a further embodiment, each single stranded region is between 3 nucleotides and 12 nucleotides in length, or between 3 nucleotides and 7 nucleotides in length, or between 3 nucleotides and 5 nucleotides in length. In other embodiments each single stranded region comprises at least one pyrimidine; each single stranded region comprises all pyrimidines; each single stranded region comprises at least one deoxycytosine; and/or each single stranded region comprises all deoxycytosines.

In various further embodiments, one or more phosphorothioate linkages between nucleotides at 1, 2, 3, 4, 5, 6, 7, or 8 boundaries of the single stranded region and the double stranded region when formed; one or more phosphorothioate linkages between nucleotides at all boundaries of the single stranded region and the double stranded region when formed; one or more phosphorothioate linkages between nucleotides within one or more of the single stranded regions; and/or there are phosphorothioate linkages in the double-stranded regions when formed other than at the one or more boundaries of the single stranded region and the double stranded region, when formed.

In one embodiment, the double stranded regions, when formed, have a GC nucleotide content of at least 10%, 20%, 30%, 40%, 50%, or more. In another embodiment, the double stranded regions when formed have a GC nucleotide content of 20% or less, 15% or less, 10% or less, 9% or less, 8% or less, 7% or less, 6% or less, 5% or less, 4% or less, 3% or less, 2% or less, 1% or less, or 0%.

In various other embodiments, 1, 2, 3, 4, or more of each single stranded region comprise one or more deoxyuracil residue; each single stranded region comprise one or more deoxyuracil residue; the one or more deoxyuracil residues are present at the residue in the single stranded region that abuts the double stranded region; and/or a deoxyuracil residues is present in the middle of 1, 2, 3, 4, or more of the single stranded region.

In other embodiments, 1, 2, 3, 4, or more of each single stranded region comprise one or more abasic site; each single stranded region comprise one or more abasic site; the one or more abasic sites are present at the residue in the single stranded region that abuts the double stranded region, when formed, and/or an abasic site is present in the middle of 1, 2, 3, 4, or more of the single stranded regions.

In a specific embodiment of the kits of the disclosure,

(i) the first polynucleotide and the second polynucleotide are of equal length and, when base-paired, form a perfectly double stranded region of between 55 contiguous base pairs in length and 75 contiguous base pairs, or between 55 contiguous base pairs in length and 70 contiguous base pairs, or between 60 contiguous base pairs in length and 75 contiguous base pairs, or between 60 contiguous base pairs in length and 70 contiguous base pairs, or 55, 56, 57, 58, 59, 60, 61, 62, 63, 64, 65, 66, 67, 68, 69, 70, 71, 72, 73, 74, or 75 contiguous base pairs;

(ii) GC; content in the double stranded region, when formed, is 20% or less, 15% or less, 10% or less, 9% or less, 8% or less, 7% or less, 6% or less, 5% or less, 4% or less, 3% or less, 2% or less, 1% or less, or 0%;

(iii) the first polynucleotide comprises a single stranded region at the 5′ end and the 3′ end, wherein each single stranded region is between 3 nucleotides and 7 nucleotides in length or between 3 nucleotides and 5 nucleotides in length;

(iv) the second polynucleotide comprises a single stranded region at the 5′ end and the 3′ end, wherein each single stranded region is between 3 nucleotides and 7 nucleotides in length or between 3 nucleotides and 5 nucleotides in length;

(v) there are no phosphorothioate-linkages between residues that are each in the double stranded regions, when formed;

(v) wherein one or more of the following is true:
(A) each single stranded region comprises all pyrimidines, preferably comprising all deoxycytosines, and there is a phosphorothioate linkages between nucleotides at all boundaries of the single stranded region and the double stranded region;
(B) each single stranded region comprises one or more abasic site, wherein (i) abasic sites are present at the residue in each single stranded region that abuts the double stranded region, and/or (ii) an abasic site is present in the middle of each of the single stranded regions.

In further embodiments, the polynucleotides of the kits comprise a combination or pair of polynucleotides as described above for the constructs of the disclosure.

### EXAMPLES

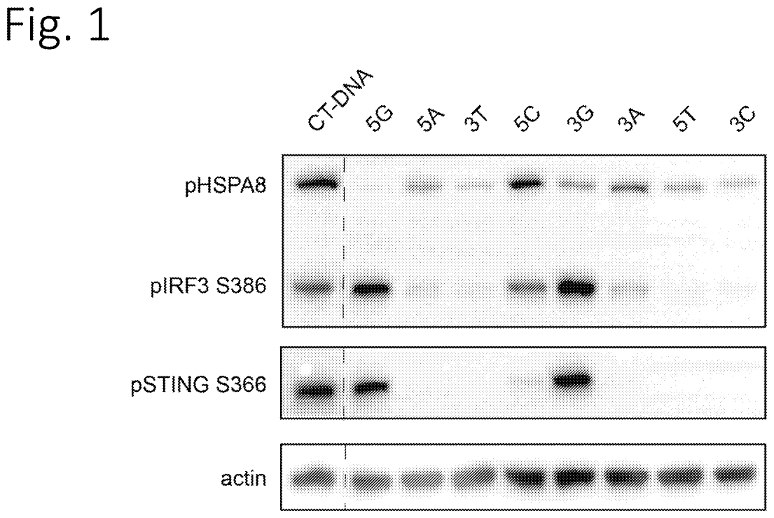

FIG. 1 shows that nucleotide content of DNA overhangs alters DNA-PK activation. Double-stranded substrates were designed to contain 35 base pairs of complementary DNA and either 3 bases or 5 bases of unpaired overhangs on both ends (Y-form DNA): these overhangs were mononucleotide in nature, comprising all As, Ts, Cs, or Gs (exact sequences listed below, with unpaired overhangs underlined). Duplex DNAs were annealed and transfected into primary human fibroblasts at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize the following phosphorylation events:

Phosphorylated HSPA8 (Ser638) is a readout for DNA-PK/SIDSP activation. Phosphorylated IRF3 (Ser386) is a readout for both cGAS/STING and DNA-PK/SIDSP activation. Phosphorylated STING (Ser366) is a readout for cGAS/STING activation.

### Sequences Tested (FIG. 1):

5T:
(SEQ ID NO: 226)
TTTTTAGGTCCCACCAGGGTCCACCTGGGGTCACCTGGGATTTTT/
(SEQ ID NO: 227)
TTTTTTCCCAGGTGACCCCAGGTGGACCCTGGTGGGACCTTTTTT
5G:
(SEQ ID NO: 228)
GGGGGAGGTCCCACCAGGGTCCACCTGGGGTCACCTGGGAGGGGG/
(SEQ ID NO: 229)
GGGGGTCCCAGGTGACCCCAGGTGGACCCTGGTGGGACCTGGGGG
3T:
(SEQ ID NO: 230)
TTTAGGTCCCACCAGGGTCCACCTGGGGTCACCTGGGATTT/
(SEQ ID NO: 231)
TTTTCCCAGGTGACCCCAGGTGGACCCTGGTGGGACCTTTT
3G:
(SEQ ID NO: 232)
GGGAGGTCCCACCAGGGTCCACCTGGGGTCACCTGGGAGGG/
(SEQ ID NO: 233)
GGGTCCCAGGTGACCCCAGGTGGACCCTGGTGGGACCTGGG
5A:
(SEQ ID NO: 1)
AAAAAAGGTCCCACCAGGGTCCACCTGGGGTCACCTGGGAAAAAA/
(SEQ ID NO: 2)
AAAAATCCCAGGTGACCCCAGGTGGACCCTGGTGGGACCTAAAAA
5C:
(SEQ ID NO: 3)
CCCCCAGGTCCCACCAGGGTCCACCTGGGGTCACCTGGGACCCCC/
(SEQ ID NO: 4)
CCCCCTCCCAGGTGACCCCAGGTGGACCCTGGTGGGACCTCCCCC
3A:
(SEQ ID NO: 5)
AAAAGGTCCCACCAGGGTCCACCTGGGGTCACCTGGGAAAA/
(SEQ ID NO: 6)
AAATCCCAGGTGACCCCAGGTGGACCCTGGTGGGACCTAAA
3C:
(SEQ ID NO: 7)
CCCAGGTCCCACCAGGGTCCACCTGGGGTCACCTGGGACCC/
(SEQ ID NO: 8)
CCCTCCCAGGTGACCCCAGGTGGACCCTGGTGGGACCTCCC

From this experiment, we conclude that Y-form DNA substrates activate the SIDSP, with pyrimidine-containing overhangs activating DNA-PK more potently than purine-containing overhangs.

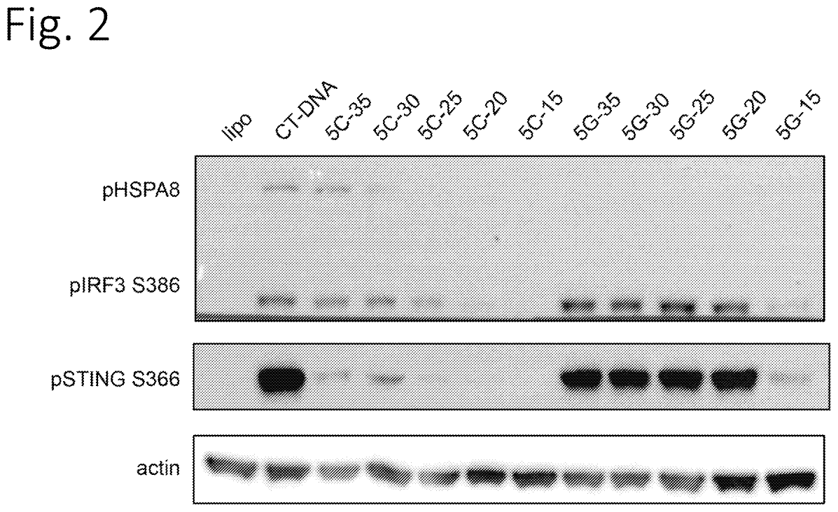

FIG. 2 shows that DNA-PK activation is dependent upon the size of the DNA ligands. Double-stranded substrates were designed to contain 15, 20, 25, 30, or 35 base pairs of complementary DNA and 5 bases of unpaired cytosine-containing overhangs on both ends (Y-form DNA). Duplex DNAs were annealed and transfected into primary human fibroblasts at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize the following phosphorylation events:

Phosphorylated HSPA8 (Ser638) is a readout for DNA-PK/SIDSP activation. Phosphorylated IRF3 (Ser386) is a readout for both cGAS % STING and DNA-PK/SIDSP activation. Phosphorylated STING (Ser366) is a readout for cGAS/STING activation.

### Sequences Tested (FIG. 2):

5C-35:
(SEQ ID NO: 3)
CCCCCAGGTCCCACCAGGGTCCACCTGGGGTCACCTGGGACCCCC/
(SEQ ID NO: 4)
CCCCCTCCCAGGTGACCCCAGGTGGACCCTGGTGGGACCTCCCCC
5C-30:
(SEQ ID NO: 236)
CCCCCACACCAGGGTCCACCTGGGGTCACCTGGGACCCCC/
(SEQ ID NO: 237)
CCCCCTCCCAGGTGACCCCAGGTGGACCCTGGTGTCCCCC
5C-25:
(SEQ ID NO: 238)
CCCCCAGGGTCCACCTGGGGTCACCTGGGACCCCC/
(SEQ ID NO: 239)
CCCCCTCCCAGGTGACCCCAGGTGGACCCTCCCCC
5C-20:
(SEQ ID NO: 240)
CCCCCACACCTGGGGTCACCTGGGACCCCC/
(SEQ ID NO: 241)
CCCCCTCCCAGGTGACCCCAGGTGTCCCCC
5C-15:
(SEQ ID NO: 242)
CCCCCTGGGGTCACCTGGGACCCCC/
(SEQ ID NO: 243)
CCCCCTCCCAGGTGACCCCACCCCC
5G-35:
(SEQ ID NO: 228)
GGGGGAGGTCCCACCAGGGTCCACCTGGGGTCACCTGGGAGGGGG/
(SEQ ID NO: 229)
GGGGGTCCCAGGTGACCCCAGGTGGACCCTGGTGGGACCTGGGGG
5G-30:
(SEQ ID NO: 246)
GGGGGACACCAGGGTCCACCTGGGGTCACCTGGGAGGGGG/
(SEQ ID NO: 247)
GGGGGTCCCAGGTGACCCCAGGTGGACCCTGGTGTGGGGG
5G-25:
(SEQ ID NO: 248)
GGGGGAGGGTCCACCTGGGGTCACCTGGGAGGGGG/
(SEQ ID NO: 249)
GGGGGTCCCAGGTGACCCCAGGTGGACCCTGGGGG
5G-20:
(SEQ ID NO: 250)
GGGGGACACCTGGGGTCACCTGGGAGGGGG/
(SEQ ID NO: 251)
GGGGGTCCCAGGTGACCCCCAGGTGTGGGGG
5G-15:
(SEQ ID NO: 252)
GGGGGTGGGGTCACCTGGGAGGGGG/
(SEQ ID NO: 253)
GGGGGTCCCAGGTGACCCCAGGGGG

From this experiment, we conclude that Y-form DNA substrates need to be at least 30 nucleotides to activate both cGAS/STING and the DNA-PK/SIDSP.

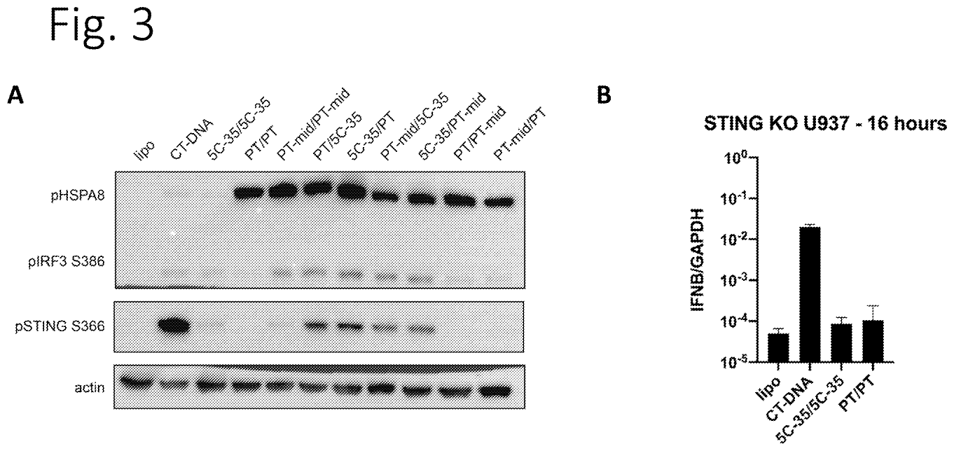

FIG. 3 shows that 35 bp phosphorothioate (PT) substrates are potent activators of DNA-PK, but do not induce IFNB via the SIDSP. Double-stranded substrates were designed to contain 35 base pairs of complementary DNA and 5 bases of unpaired cytosine-containing overhangs on both ends (Y-form DNA). Substrates were also designed with either canonical phosphodiester linkages between bases, or phosphorothioate linkages (PT). A) Duplex DNAs were annealed and transfected into primary human fibroblasts at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize the following phosphorylation events:

Phosphorylated HSPA8 (Ser638) is a readout for DNA-PK/SIDSP activation. Phosphorylated IRF3 (Ser386) is a readout for both cGAS/STING and DNA-PK/SIDSP activation. Phosphorylated STING (Ser366) is a readout for cGAS/STING activation. B) Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate.

Sequences tested (FIG. 3) (where asterisks between bases denote individual phosphorothioate (PT) linkages):

5C-35 top:
(SEQ ID NO: 3)
CCCCCAGGTCCCACCAGGGTCCACCTGGGGTCACCTGGGACCCCC/
5C-35 bottom:
(SEQ ID NO: 4)
CCCCCTCCCAGGTGACCCCAGGTGGACCCTGGTGGGACCTCCCCC
PT top:
(SEQ ID NO: 256)
CCCCCA*G*G*T*C*C*C*A*C*C*A*G*G*G*T*C*C*A*C*C*T*G*G*G*G*T*C*A*C
*C*T*G*G*G*ACCCCC
PT bottom:
(SEQ ID NO: 257)
CCCCCT*C*C*C*A*G*G*T*G*A*C*C*C*C*A*G*G*T*G*G*A*C*C*C*T*G*G*T*G
*G*G*A*C*C*TCCCCC
PT mid top:
(SEQ ID NO: 258)
CCCCCAGGTCCCACCAG*G*G*T*C*C*A*C*C*T*G*G*G*GTCACCTGGGACCCCC
PT mid bottom:
(SEQ ID NO: 259)
CCCCCTCCCAGGTGAC*C*C*C*C*A*G*G*T*G*G*A*C*C*CTGGTGGGACCTCCCCC

From this experiment, we conclude that phosphorothioate-containing substrates are potent activators of DNA-PK (as read out by HSPA8 phosphorylation); however, these 35 bp sequences (both phosphodiester-(5C-35) and phosphorothioate (PT)-containing) are insufficient to stimulate the production of IFNB.

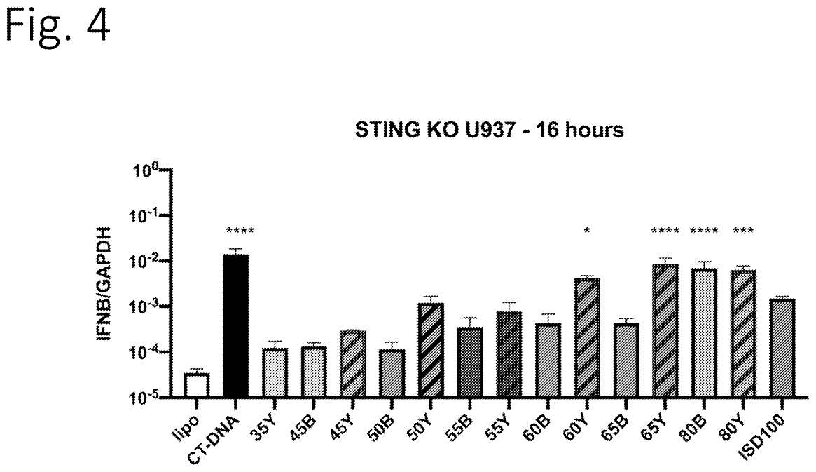

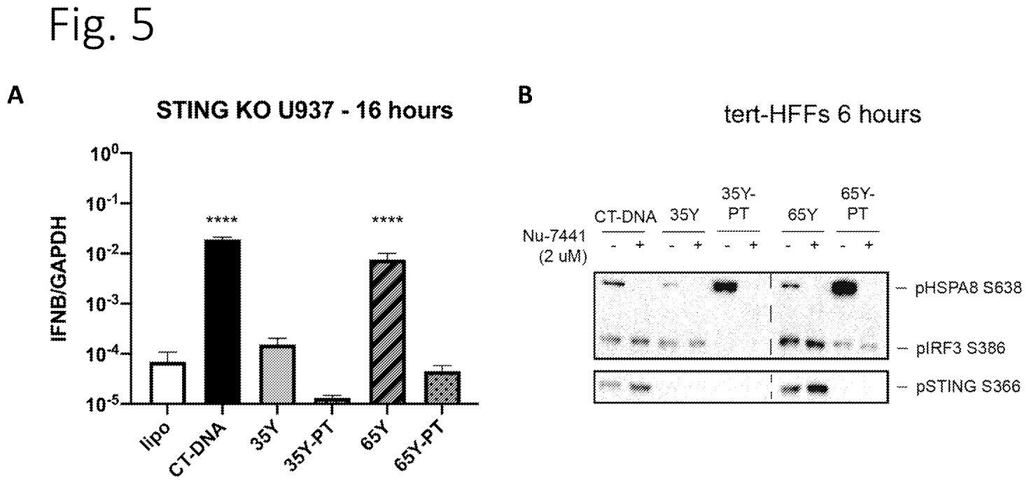

FIG. 4 shows that longer (>=50 bp) double-stranded oligonucleotide substrates stimulate IFNB production via the SIDSP. Duplex oligos were designed either as blunt (B) or Y-form (Y) substrates (with 5 bp C-containing overhangs), of length 35, 45, 50, 55, 60, 65, and 80 bp, such that the length denoted refers to the length of the complementary, double-stranded regions and excludes the single-stranded overhangs (in the case of Y-form DNA). All duplex DNAs were tested against ISD100, a 100 bp blunt dsDNA. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Sequences Tested (FIG. 4):

35Y:
(SEQ ID NO: 3)
CCCCCAGGTCCCACCAGGCTCCACCTGGGGTCACCTGGGACCCCC/
(SEQ ID NO: 4)
CCCCCTCCCAGGTGACCCCAGGTGGACCCTGGTGGGACCTCCCCC
45B:
(SEQ ID NO: 262)
TAGTGATTATCTAGACATACATCTAGTACATGTCTAGTCAGTATA/
(SEQ ID NO: 263)
TATACTGACTAGACATGTACTAGATGTATGTCTAGATAATCACTA
50B:
(SEQ ID NO: 264)
ATCTAGTGATTATCTAGACATACATCTAGTACATGTCTAGTCAGTATCTA/
(SEQ ID NO: 265)
TAGATACTGACTAGACATGTACTAGATGTATGTCTAGATAATCACTAGAT
45Y;
(SEQ ID NO: 9)
CCCCCTAGTGATTATCTAGACATACATCTAGTACATGTCTAGTCAGTATACCCCC/
(SEQ ID NO: 10)
CCCCCTATACTGACTAGACATGTACTAGATGTATGTCTAGATAATCACTACCCCC
50Y:
(SEQ ID NO: 11)
CCCCCATCTAGTGATTATCTAGACATACATCTAGTACATGTCTAGTCAGTATCTACCCCC/
(SEQ ID NO: 12)
CCCCCTAGATACTGACTAGACATGTACTAGATGTATGTCTAGATAATCACTAGATCCCCC
55B:
(SEQ ID NO: 13)
AGTATCTAGTGATTATCTAGACATACATCTAGTACATGTCTAGTCAGTATCTAGT/
(SEQ ID NO: 14)
ACTAGATACTGACTAGACATGTACTAGATGTATGTCTAGATAATCACTAGATACT
55Y:
(SEQ ID NO: 15)
CCCCCAGTATCTAGTGATTATCTAGACATACATCTAGTACATGTCTAGTCAGTATCTAGTCC
CCC/
(SEQ ID NO: 16)
CCCCCACTAGATACTGACTAGACATGTACTAGATGTATGTCTAGATAATCACTAGATACTCC
CCC
60B:
(SEQ ID NO: 17)
TCAGTATCTAGTGATTATCTAGACATACATCTAGTACATGTCTAGTCAGTATCTAGTGAT/
(SEQ ID NO: 18)
ATCACTAGATACTGACTAGACATGTACTAGATGTATGTCTAGATAATCACTAGATACTGA
60Y:
(SEQ ID NO: 19)
CCCCCTCAGTATCTAGTGATTATCTAGACATACATCTAGTACATGTCTAGTCAGTATCTAGT
GATCCCCC/
(SEQ ID NO: 20)
CCCCCATCACTAGATACTGACTAGACATGTACTAGATGTATGTCTAGATAATCACTAGATA
CTGACCCCC
65Y:
(SEQ ID NO: 21)
CCCCCAGTCAGTATCTAGTGATTATCTAGACATACATCTAGTACATGTCTAGTCAGTA
TCTAGTGATTATCCCCC/
(SEQ ID NO: 22)
CCCCCATAATCACTAGATACTGACTAGACATGTACTAGATGTATGTCTAGATAATCACTAGA
TACTGACTCCCCC
65B:
(SEQ ID NO: 23)
AGTCAGTATCTAGTGATTATCTAGACATACATCTAGTACATGTCTAGTCAGTATCTAGTGAT
TAT/
(SEQ ID NO: 24)
ATAATCACTAGATACTGACTAGACATGTACTAGATGTATGTCTAGATAATCACTAGATACTG
ACT
80Y:
(SEQ ID NO: 25)
CCCCCAATGTCTAGTCAGTATCTAGTGATTATCTAGACATACATCTAGTACATGTCTAGTCA
GTATCTAGTGATTATCTAGACATCCCCC/
(SEQ ID NO: 26)
CCCCCATGTCTAGATAATCACTAGATACTGACTAGACATGTACTAGATGTATGTCTAGATAA
TCACTAGATACTGACTAGACATTCCCCC
80B:
(SEQ ID NO: 27)
AATGTCTAGTCAGTATCTAGTGATTATCTAGACATACATCTAGTACATGTCTAGTCAGTATC
TAGTGATTATCTAGACAT/
(SEQ ID NO: 28)
ATGTCTAGATAATCACTAGATACTGACTAGACATGTACTAGATGTATGTCTAGAT
AATCACTAGATACTGAOTAGACATT
ISD100:
(SEQ ID NO: 29)
ACATCTAGTACATGTCTAGTCAGTATCTAGTGATTATCTAGACATACATCTAGTACATGTCT
AGTCAGTATCTAGTGATTATCTAGACATGGACTCATCC/
(SEQ ID NO: 30)
GGATGAGTCCATGTCTAGATAATCACTAGATACTGACTAGACATGTACTAGATGT
ATGTCTAGATAATCACTAGATACTGACTAGACATGTACTAGATGT

From this experiment, we conclude that SIDSP activation requires >=45 bp double-stranded DNA, with the optimal length at 65 base pairs. Also, both blunt and Y-form DNA can activate the SIDSP, though Y-form is a more potent agonist, especially for shorter ligands.

FIG. 5 shows that 65 bp phosphodiester ligands stimulate IFNB production via the SIDSP, but phosphorothioate ligands of the same size do not.

Duplex oligos were designed as Y-form (Y) 65 bp substrates with 5 bp C-containing overhangs, containing either phosphodiester linkages or phosphorothioate (PT) linkages across the double-stranded portion of the ligand. A) Duplex DNAs were annealed and transfected into STING deficient U937 cells at 4 ug/m final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate. B) Duplex DNAs were annealed and transfected into tert-immortalized human foreskin fibroblasts (pretreated with either DMSO or the DNA-PK inhibitor Nu-7441 at 2 uM) at 4 ug/mL final concentration using lipofectamine reagent. After 6 hours, cells were harvested in RIPA buffer and lysates were run through gel electrophoresis. Proteins were transferred to nitrocellulose membranes, blocked with 5% BSA in TBST, washed, and blotted with antibodies to recognize the following phosphorylation events:

Phosphorylated HSPA8 (Ser638) is a readout for DNA-PK/SIDSP activation. Phosphorylated IRF3 (Ser386) is a readout for both cGAS/STING and DNA-PK/SIDSP activation. Phosphorylated STING (Ser366) is a readout for cGAS/STING activation.

Sequences tested (FIG. 5) (where asterisks between bases denote individual phosphorothioate (PT) linkages):

35Y:
(SEQ ID NO: 3)
CCCCCAGGTCCCACCAGGGTCCACCTGGGGTCACCTGGGACCCCC/
(SEQ ID NO: 4)
CCCCCTCCCAGGTGACCCCAGGTGGACCCTGGTGGGACCTCCCCC
35Y-PT:
(SEQ ID NO: 256)
CCCCCA*G*G*T*C*C*C*A*C*C*A*G*G*G*T*C*C*A*C*C*T*G*G*G*G*T*C*A*C*
*C*T*G*G*G*ACCCCC/
(SEQ ID NO: 257)
CCCCCT*C*C*C*A*G*G**G*A*C*C*C*C*A*G*GT*G*G*A*C*C*C*T
*G*G*T*G*G*G*A*C*C*TCCCCC
65Y-PT:
(SEQ ID NO: 266)
CCCCCA*G*T*C*A*G*T*A*T*C*T*A*G*T*G*A*T*T*A*T*C*T*A*G*A*C*A*T*A
*C*A*T*C*T*A*G*T*A*C*A*T*G*T*C*T*A*G*T*C*A*G*T*A*T*C*T*A*G*T*G
*A*T*T*A*TCCCCC/
(SEQ ID NO: 267)
CCCCCA*T*A*A*T*C*A*C*T*A*G*A*T*A*C*T*G*A*C*T*A*G*A*C*A*T*G*T*A
*C*T*A*G*A*T*G*T*A*T*G*T*C*TA*G*A*T*A*A*T*C*A*C*T*A*G*A*T*A*C
*T*G*A*C*TCCCCC
65Y:
(SEQ ID NO: 21)
CCCCCAGTCAGTATCTAGTGATTATCTAGACATACATCTAGTACATGTCTAGTCAGTA
TCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC

From this experiment, we conclude that while 65 bp Y-form DNA activates the SIDSP, including phosphorothioate linkages across the double-stranded portion of the substrate fails to promote IFNB production via the SIDSP. However, both phosphodiester- and phosphorothioate-containing 65Y substrates activate DNA-PK (strong pHSPA8 that is sensitive to DNA-PK inhibitor).

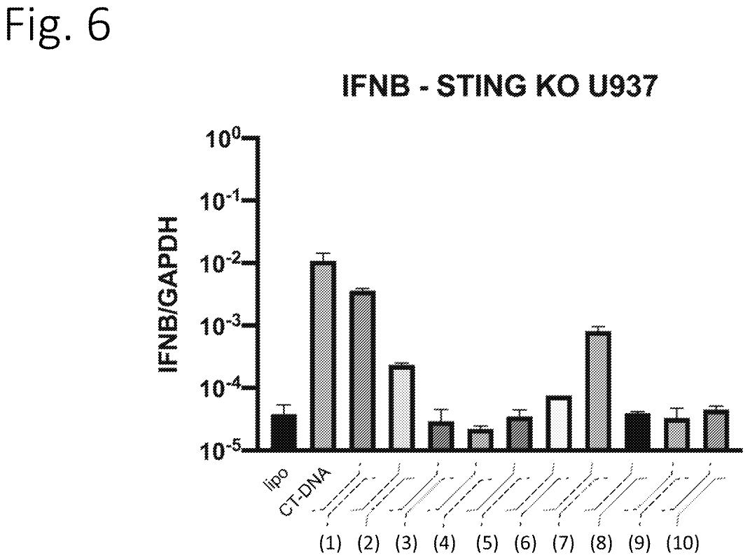

FIG. 6 shows that phosphorothioate linkages in the middle of one or both strands of 65Y ligands inhibits SIDSP activation. Duplex oligos were designed as Y-form (Y) 65 bp substrates with 5 bp C-containing overhangs, containing either phosphodiester linkages or phosphorothioate (PT) linkages as denoted by red solid lines. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was perforned in both biological and technical duplicate.

### Substrates Tested:

*in figure, PT linkages are schematized in red, solid lines, while phosphodiester linkages are schematized in black, dashed lines. In the sequences below, PT linkages are denoted by asterisks.

(3):
(SEQ ID NO: 268)
CCCCCAGTCAG*T*A*T*C*T*A*G*T*G*A*T*T*A*T*C*T*A*G*A*C*A*T*A*
C*A*T*C*T*A*G*T*A*C*A*T*G*T*C*T*A*G*T*C*A*G*T*A**C*T*A*G*T*GA
TTATCCCCC/
(SEQ ID NO: 269)
CCCCCATAATC*A*C*T*A*G*A*T*A*C*T*G*A*C*T*A*G*A*C*A*T*G*
T*A*C*T*A*G*A**G*T*A*T*G**C*T*A*G*A*T*A*A**C*A*C*T*A*G*A*T*
A*CTGACTCCCCC
(4):
(SEQ ID NO: 266)
CCCCCA*G*T*C*A*G*T*A*T*C*T*A*G*T*G*A*T*T*A*T*C*T*A*G*A*C*A
*T*A*C*A*T*C**A*G*T*A*C*A**G*T*C**A*G**C*A*G*T*A*T*C**A*G
*T*G*A*T*T*A*TCCCCC/
(SEQ ID NO: 22)
CCCCCATAATCACTAGATACTGACTAGACATGTACTAGATGTATGTCTAGATAA
TCACTAGATACTGACTCCCCC
(5):
(SEQ ID NO: 270)
C*C*C*C*C*A*G*T*C*A*G*T*A*T*C*T*A*G*T*G*A*T*T*A*T*C*T*A*G*
A*C*A**A*C*A**C**A*G*T*A*C*A*T*G**C*T*A*G*T*C*A*G*T*A**C*
T*A*G*T*G*A*T*T*A*T*C*C*C*C*C/
(SEQ ID NO: 22)
CCCCCATAATCACTAGATACTGACTAGACATGTACTAGATGTATGTCTAGATAA
TCACTAGATACTGACTCCCCC
(6):
(SEQ ID NO: 35)
C*C*C*C*C*A*G*T*C*A*GTATCTAGTGATTATCTAGACATACATCTAGTACATGT
CTAGTCAGTATCTAGTG*A*T*T*A*T*C*C*C*C*C/
(SEQ ID NO: 269)
CCCCCATAATC*A*C*T*A*G*A*T*A*C*T*G*A*C*T*A*G*A*C*A*T*G*T
*A*C*T*A*G*A**G*T*A*T*G*T*C*T*A*G*A**A*A**C*A*C*T*A*G*A*T*A
*CTGACTCCCCC
(8):
(SEQ ID NO: 35)
C*C*C*C*C*A*G*T*C*A*GTATCTAGTGATTATCTAGACATACATCTAGTACATGT
CTAGTCAGTATCTAGTG*A*T*T*A*T*C*C*C*C*C/
(SEQ ID NO: 271)
C*C*C*C*C*A*T*A*A*T*C*A*C*T*A*G*A*T*A*C*T*G*A*C*P*A*G*A
*C*A**G*T*A*C*T*A*G*A*T*G**A**G**C*T*A*G*A*T*A*A*T*C*A*C*T
*A*G*A*T*A*C*T*G*A*C*T*C*C*C*C*C
(9):
(SEQ ID NO: 268)
CCCCCAGTCAG*T*A*T*C*T*A*G*T*G*A*T*T*A*T*C*T*A*G*A*C*A*T*A*
C*A*T*C**A*G**A*C*A**G*T*C**A*G**C*A*G*T*A**C**A*G*T*GA
TTATCCCCC/
(SEQ ID NO: 22)
CCCCCATAATCACTAGATACTGACTAGACATGTACTAGATGTATGTCTAGATAA
TCACTAGATACTGACTCCCCC
(10):
(SEQ ID NO: 268)
CCCCCAGTCAG*T*A*T*C*T*A*G*T*G*A*T*T*A*T*C*T*A*G*A*C*A*T*A
*C*A*T*C**A*G*T*A*C*A**G**C**A*G*T*C*A*G*T*A**C*T*A*G*T*G
ATTATCCCCC/
(SEQ ID NO: 271)
C*C*C*C*C*A*T*A*A*T*C*A*C*T*A*G*A*T*A*C*T*G*A*C*T*A*G*
A*C*A-*G*T*A*C*T*A*G*A*T*G*T*A*T*G*T*C*T*A*G*A*-A*A*T*C*A*C*
T*A*G*A*T*A*C*T*G*A*C*T*C*C*C*C*C
(1):
(SEQ ID NO: 21)
CCCCCAGTCAGTATCTAGTGATTATCTAGACATACATCTAGTACATGTCTAGTCAGTA
TCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(2):
(SEQ ID NO: 35)
C*C*C*C*C*A*G*T*C*A*GTATCTAGTGATTATCTAGACATACATCTAGTACATGT
CTAGTCAGTATCTAGTG*A*T*T*A*T*C*C*C*C*C/
(SEQ ID NO: 36)
C*C*C*C*C*A*T*A*A*T*CACTAGATACTGACTAGACATGTACTAGATGTATG
TCTAGATAATCACTAGATAC*T*G*A*C*T*C*C*C*C*C
(7):
(SEQ ID NO: 35)
C*C*C*C*C*A*G*T*C*A*GTATCTAGTGATTATCTAGACATACATCTAGTACATGT
CTAGTCAGTATCTAGTG*A*T*T*A*T*C*C*C*C*C/
(SEQ ID NO: 22)
CCCCCATAATCACTAGATACTGACTAGACATGTACTAGATGTATGTCTAGATAAT
CACTAGATACTGACTCCCCC

From this experiment, we conclude that the presence of PT linkages in the middle of one or both strands of the substrate inhibits activation of the SIDSP/production of IFNB. The two conditions in which we see “intermediate” phenotypes (IFNB signal between lipo and (1)) are (2) and (8), which include only phosphodiester linkages in the middle of both strands of the substrate.

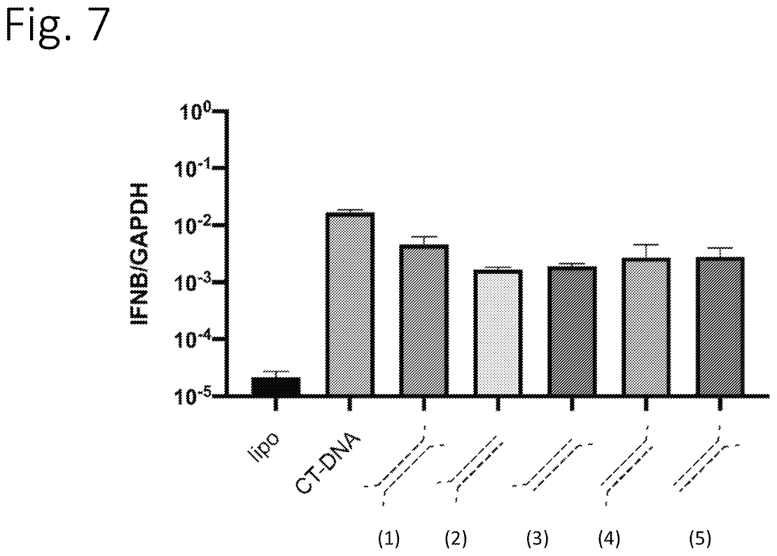

FIG. 7 shows that highest SIDSP activation is achieved with Y-form DNA, including both 5′ and 3′ overhangs on each end. Duplex oligos were designed as 65 bp substrates with either 5′, 3′, or single-sided 5C-containing overhangs. A) Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate.

### Substrates Tested:

(where underlined residues represent unpaired overhang regions)

(1):
(SEQ ID NO: 21)
CCCCCAGTCAGTATCTAGTGATTATCTAGACATACATCTAGTACATGTCTAGTCAGTA
TCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(2):
(SEQ ID NO: 41)
CCCCCAGTCAGTATCTAGTGATTATCTAGACATACATCTAGTACATGTCTAGTCAGTA
TCTAGTGATTAT/
(SEQ ID NO: 42)
ATAATCACTAGATACTGACTAGACATGTACTAGATGTATGTCTAGATAATCACTA
GATACTGACTCCCCC
(3):
(SEQ ID NO: 41)
CCCCCAGTCAGTATCTAGTGATTATCTAGACATACATCTAGTACATGTCTAGTCAGTA
TCTAGTGATTAT/
(SEQ ID NO: 44)
CCCCCATAATCACTAGATACTGACTAGACATGTACTAGATGTATGTCTAGATAAT
CAGTAGATAGTGACT
(4):
(SEQ ID NO: 45)
AGTCAGTATCTAGTGATTATCTAGACATACATCTAGTACATGTCTAGTCAGTATCTAG
TGATTATCCCCC/
(SEQ ID NO: 42)
ATAATCACTAGATACTGACTAGACATGTACTAGATGTATGTCTAGATAATCACTA
GATACTGACTCCCCC
(5):
(SEQ ID NO: 45)
AGTCAGTATCTAGTGATTATCTAGACATACATCTAGTACATGTCTAGTCAGTATCTAG
TGATTATCCCCC/
(SEQ ID NO: 44)
CCCCCAAATCACAGATACTGACTAGACATGTACTAGATGTATGTCTAGATAAT
CACTAGATACTGACT

From this experiment, we conclude that potent SIDSP activation can be achieved with either 5′ or 3′ overhangs, or with overhangs only on one side, however, the most potent SIDSP activation is achieved with Y-form DNA ((1)), in which both 5′ and 3′ unpaired overhangs are present on both sides of the substrate.

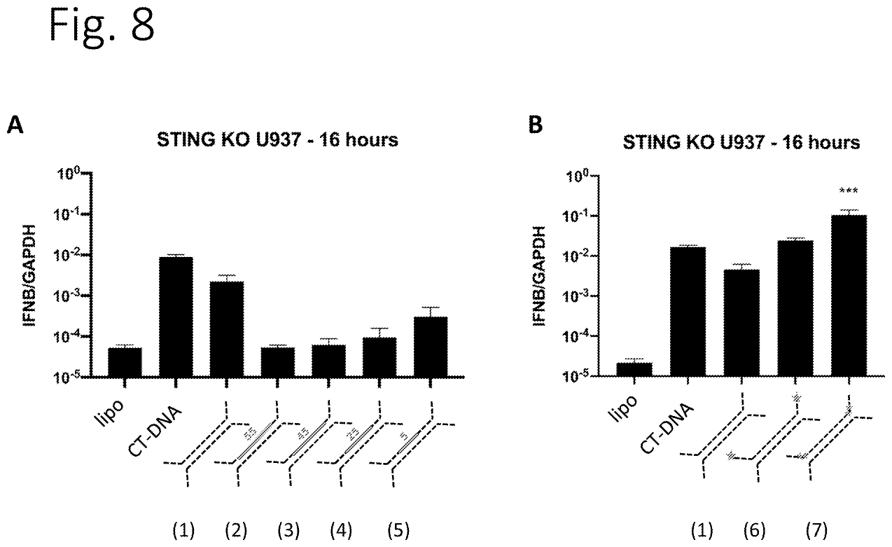

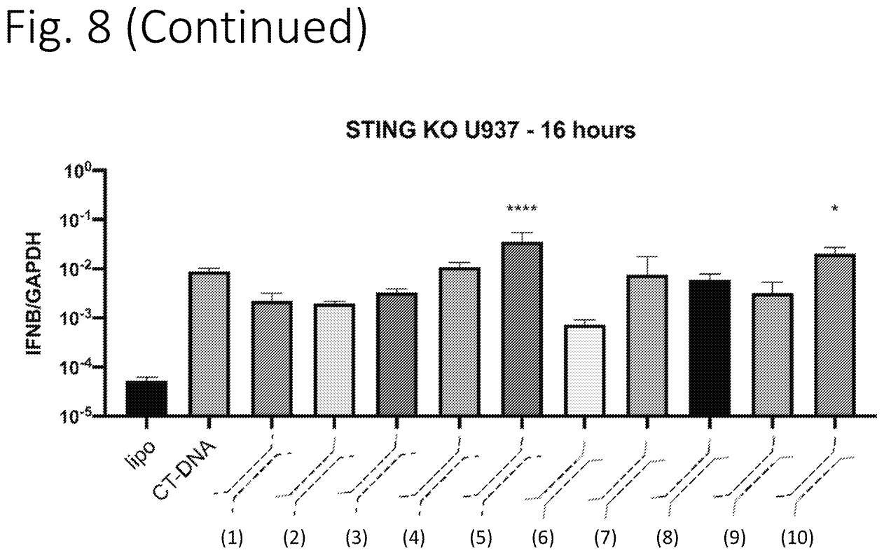

FIG. 8 shows that PT linkages display drastic position dependent effects, in which “middle” PT linkages inhibit SIDSP activation, whereas PT linkages at the end or the single-/double-stranded “boundary” region of the substrate promotes potent SIDSP activation. Duplex oligos were designed as Y-form (Y) 65 bp substrates with 5 bp C-containing overhangs, containing either phosphodiester linkages as denoted by black dashed lines or phosphorothioate (PT) linkages as denoted by red solid lines or red stars. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate.

Substrates Tested:

*in figure, PT linkages are schematized in red, solid lines (or dots), while phosphodiester linkages are schematized in black, dashed lines. In the sequences below, PT linkages are denoted by asterisks.

### 8A and 8B:

(2):
(SEQ ID NO: 268)
CCCCCAGTCAG*T*A*T*C*T*A*G*T*G*A*T*T*A*T*C*T*A*G *A*C*A*T*A*
C*A*T*C* T*A*G*T*A*C *A**G**0**A*G**C*A*G*T*A*T*C **A*G*T*GA
TTATCCCCC/
(SEQ ID NO: 22)
CCCCCATAATCACTAGATACTGACTAGACATGTACTAGATGTATGTCTAGATAA
TCACTAGATACTGACTCCCCC
(3):
(SEQ ID NO: 272)
CCCCCAGTCAGTATCT*A*G*T*G*A*T*T*A*T*C*T*A*G*A*C*A*T*A*C*A*T
*C*T*A*G*T*A*C*A*T*G*T*C*T*A*G*T*C*A*G*T*A*T*C*TAGTGATTATCCCCC/
(SEQ ID NO: 22)
CCCCCATAATCACTAGATACTGACTAGACATGTACTAGATGTATGTCTAGATAA
TCACTAGATACTGACTCCCCC
(4):
(SEQ ID NO: 273)
CCCCCAGTCAGTATCTAGTGATTATC*T*A*G*A*C*A*T*A*C*A*T*C*T*A*G*T
*A*C*A*T*G*T*C*T*AGTCAGTATCTAGTGATTATCCCCC/
(SEQ ID NO: 22)
CCCCCATAATCACTAGATACTGACTAGACATGTACTAGATGTATGTCTAGATAA
TCACTAGATACTGACTCCCCC
(1):
(SEQ ID NO: 21)
CCCCCAGTCAGTATCTAGTGATTATCTAGACATACATCTAGTACATGTCTAGTCAGTA
TCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(5):
(SEQ ID NO: 51)
CCCCCAGTCAGTATCTAGTGATTATCTAGACATACA*T*C*T*A*GTACATGTCTAGT
CAGTATCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(6):
(SEQ ID NO: 53)
C*CCCCAGTCAGTATCTAGTGATTATCTAGACATACATCTAGTACATGTCTAGTCAGT
ATCTAGTGATTATCCCC*C/
(SEQ ID NO: 22)
CCCCCATAATCACTAGATACTGACTAGACATGTACTAGATGTATGTCTAGATAAT
CACTAGATACTGACTCCCCC
(7):
(SEQ ID NO: 55)
CCCCC*A*GTCAGTATCTAGTGATTATCTAGACATACATCTAGTACATGTCTAGTCAG
TATCTAGTGATTA*T*CCCCC/
(SEQ ID NO: 22)
CCCCCATAATCACTAGATACTGACTAGACATGTACTAGATGTATGTCTAGATAAT
CACTAGATACTGACTCCCCC
8C:
(1):
(SEQ ID NO: 21)
CCCCCAGTCAGTATCTAGTGATTATCTAGACATACATCTAGTACATGTCTAGTCAGTA
TCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(2):
(SEQ ID NO: 59)
C*C*C*C*C*AGTCAGTATCTAGTGATTATCTAGACATACATCTAGTACATGTCTAGT
CAGTATCTAGTGATTAT*C*C*C*C*C/
(SEQ ID NO: 22)
CCCCCATAATCACTAGATACTGACTAGACATGTACTAGATGTATGTCTAGATAAT
CACTAGATACTGACTCCCCC
(3):
(SEQ ID NO: 61)
C*C*C*CCAGTCAGTATCTAGTGATTATCTAGACATACATCTAGTACATGTCTAGTCA
GTATCTAGTGATTATCC*GC*C/
(SEQ ID NO: 22)
CCCCCAAATCACAGATACTGACTAGACATGTACTAGATGTATGTCTAGATAAT
CACTAGATACTGACTCCCCC
(4):
(SEQ ID NO: 53)
C*CCCCAGTCAGTATCTAGTGATTATCTAGACATACATCTAGTACATGTCTAGTCAGT
ATCTAGTGATTATCCCC*C/
(SEQ ID NO: 22)
CCCCCATAATCACTAGATACTGACTAGACATGTACTAGATGTATGTCTAGATAAT
CACTAGATACTGACTCCCCC
(5):
(SEQ ID NO: 55)
CCCCC*A*GTCAGTATCTAGTGATTATCTAGACATACATCTAGTACATGTCTAGTCAG
TATCTAGTGATTA*T*CCCCC/
(SEQ ID NO: 22)
CCCCCATAATCACTAGATACTGACTAGACATGTACTAGATGTATGTCTAGATAAT
CACTAGATACTGACTCCCCC
(6):
(SEQ ID NO: 35)
C*C*C*C*C*A*G*T*C*A*GTATCTAGTGATTATCTAGACATACATCTAGTACATGT
CTAGTCAGTATCTAGTG*A*T*T*A*T*C*C*C*C*C/
(SEQ ID NO: 36)
C*C*C*C*C*A*T*A*A*T*CACTAGATACTGACTAGACATGTACTAGATGTATG
TCTAGATAATCACTAGATAC*T*G*A*C*T*C*C*C*C*C
(7):
((SEQ ID NO: 59)
C*C*C*C*C*AGTCAGTATCTAGTGATTATCTAGACATACATCTAGTACATGTCTAGT
CAGTATCTAGTGATTAT*C*C*C*C*C/
(SEQ ID NO: 36)
C*C*C*C*C*A*T*A*A*T*CACTAGATACTGACTAGACATGTACTAGATGTATG
TCTAGATAATCACTAGATAC*T*G*A*C*T*G*C*C*C*C*C
(8):
(SEQ ID NO: 61)
C*C*C*CCAGTCAGTATCTAGTGATTATCTAGACATACATCTAGTACATGTCTAGTCA
GTATCTAGTGATTATCC*C*C*C/
(SEQ ID NO: 36)
C*C*C*C*C*A*T*A*A*T*CACTAGATACTGACTAGACATGTACTAGATGTATG
TCTAGATAATCACTAGATAC*T*G*A*C*T*C*C*C*C*C
(9):
(SEQ ID NO: 53)
C*CCCCAGTCAGTATCTAGTGATTATCTAGACATACATCTAGTACATGTCTAGTCAGT
ATCTAGTGATTATCCCC*C/
(SEQ ID NO: 36)
C*C*C*C*C*A*T*A*A*T*CACTAGATACTGACTAGACATGTACTAGATGTATG
TCTAGATAATCACTAGATAC*T*G*A*C*T*C*C*C*C*C
(10):
(SEQ ID NO: 55)
CCCCC*A*GTCAGTATCTAGTGATTATCTAGACATACATCTAGTACATGTCTAGTCA
GTATCTAGTGATTA*T*CCCCC/
(SEQ ID NO: 36)
C*C*C*C*C*A*T*A*A*T*CACTAGATACTGACTAGACATGTACTAGATGTATG
TCTAGATAATCACTAGATAC*T*G*A*C*T*C*C*C*C*C

From this experiment, we conclude that PT linkages in the middle of Y-form substrates inhibit the SIDSP, even with just 5 PT linkages (8A:(5)). However, adding PT linkages at the very end (8B:(6)) or at the single-stranded/double-stranded boundary region (8B:(7)) potentiates the SIDSP, even more so than a fully phosphodiester substrate ((I)). Boundary PT linkages also overcome limitations of less ideal substrates on the opposite strand (C:(10)).

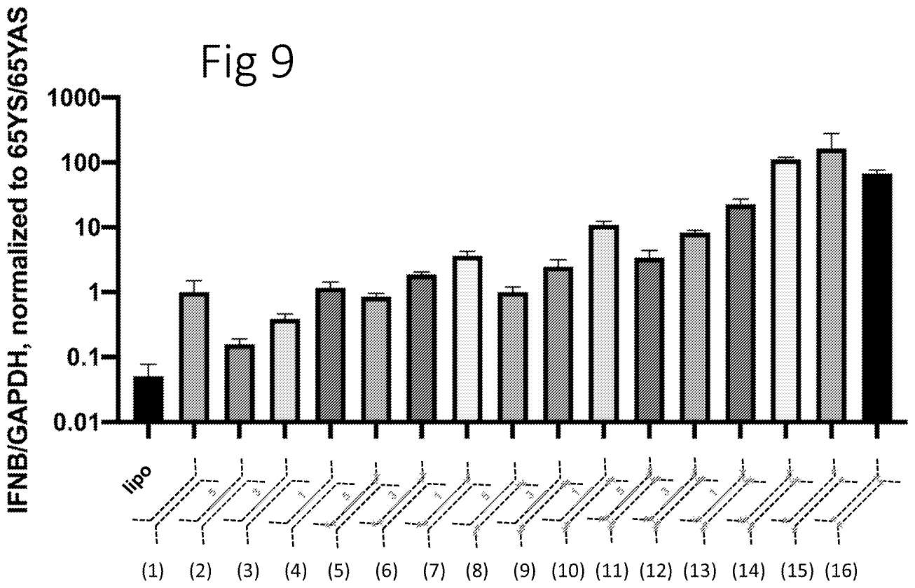

FIG. 9 further shows the independent, additive effects of PT linkages at the single-stranded/double-stranded boundary region of Y-form duplexes. Duplex oligos were designed as Y-form (Y) 65 bp substrates with 5 bp C-containing overhangs, containing either phosphodiester linkages as denoted by black dashed lines or phosphorothioate (PT) linkages as denoted by red solid lines or red stars. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in both biological and technical duplicate. This figure summarizes the result of multiple different experiments; therefore, for accurate comparisons between results from different experiments, all values were normalized to those of 65YS/65YAS (1) from their experiment and plotted on the same graph.

Substrates Tested:

*in figure, PT linkages are schematized in red, solid lines (or stars), while phosphodiester linkages are schematized in black, dashed lines. Numbers denote the number of PT linkages in the middle of the substrate, if applicable. In the sequences below, PT linkages are denoted by asterisks.

(1):
(SEQ ID NO: 21)
CCCCCAGTCAGTATCTAGTGATTATCTAGACATACATCTAGTACATGTCTAGTCAGTA
TCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(2):
(SEQ ID NO: 51)
CCCCCAGTCAGTATCTAGTGATTATCTAGACATACA*T*C*T*A*GTACATGTCTAGT
CAGTATCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(3):
(SEQ ID NO: 81)
CCCCCAGTCAGTATCTAGTGATTATCTAGACATACAT*C*T*AGTACATGTCTAGTCA
GTATCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(4):
(SEQ ID NO: 83)
CCCCCAGTCAGTATCTAGTGATTATCTAGACATACATC*TAGTACATGTCTAGTCAGT
ATCTAGTGATTATCCCCC/
(SEQ ID NO: 22)
CCCCCATAATCACTAGATACTGACTAGACATGTACTAGATGTATGTCTAGATAAT
CACTAGATACTGACTCCCCC
(5):
(SEQ ID NO: 85)
CCCCC*A*GTCAGTATCTAGTGATTATCTAGACATACA*T*C*T*A*GTACATGTCTA
GTCAGTATCTAGTGATTA*T*CCCCC/
(SEQ ID NO: 22)
CCCCCATAATCACTAGATACTGACTAGACATGTACTAGATGTATGTCTAGATAAT
CACTAGATACTGACTCCCCC
(6):
(SEQ ID NO: 87)
CCCCC*A*GTCAGTATCTAGTGATTATCTAGACATACAT*C*T*AGTACATGTCTAGT
CAGTATCTAGTGATTA*T*CCCCC/
(SEQ ID NO: 22)
CCCCCATAATCACTAGATACTGACTAGACATGTACTAGATGTATGTCTAGATAAT
CACTAGATACTGACTCCCCC
(7):
(SEQ ID NO: 89)
CCCCC*A*GTCAGTATCTAGTGATTATCTAGACATACATC*TAGTACATGTCTAGTCA
GTATCTAGTGATTA*T*CCCCC/
(SEQ ID NO: 22)
CCCCCATAATCACTAGATACTGACTAGACATGTACTAGATGTATGTCTAGATAAT
CACTAGATACTGACTCCCCC
(8):
(SEQ ID NO: 51)
CCCCCAGTCAGTATCTAGTGATTATCTAGACATACA*T*C*T*A*GTACATGTCTAGT
CAGTATCTAGTGATTATCCCCC/
(SEQ ID NO: 92)
CCCCC*A*TAATCACTAGATACTGACTAGACATGTACTAGATGTATGTCTAGATA
ATCACTAGATACTGAC*T*CCCCC
(9):
(SEQ ID NO: 81)
CCCCCAGTCAGTATCTAGTGATTATCTAGACATACAT*C*T*AGTACATGTCTAGTCA
GTATCTAGTGATTATCCCCC/
(SEQ ID NO: 92)
CCCCC*A*TAATCACTAGATACTGACTAGACATGTACTAGTACATGTCTAGTCAG
ATCACTAGATACTGAC*T*CCCCC
(10):
(SEQ ID NO: 83)
CCCCCAGTCAGTATCTAGTGATTATCTAGACATACATC*TAGTACATGTCTAGTCAG
TATCTAGTGATTATCCCCC/
(SEQ ID NO: 92)
CCCCC*A*TAATCACTAGATACTGACTAGACATGTACTAGATGTATGTCTAGATA
ATCACTAGATACTGAC*T*CCCCC
(11):
(SEQ ID NO: 85)
CCCCC*A*GTCAGTATCTAGTGATTATCTAGACATACA*T*C*T*A*GTACATGTCT
AGTCAGTATCTAGTGATTA*T*CCCCC/
(SEQ ID NO: 92)
CCCCC*A*TAATCACTAGATACTGACTAGACATGTACTAGATGTATGTCTAGATA
ATCACTAGATACTGAC*T*CCCCC
(12):
(SEQ ID NO: 87)
CCCCC*A*GTCAGTATCTAGTGATTATCTAGACATACAT*C*T*AGTACATGTCTAG
TCAGTATCTAGTGATTA*T*CCCCC/
(SEQ ID NO: 92)
CCCCC*A*TAATCACTAGATACTGACTAGACATGTACTAGATGTATGTCTAGATA
ATCACTAGATACTGAC*T*CCCCC
(13):
(SEQ ID NO: 89)
CCCCC*A*GTCAGTATCTAGTGATTATCTAGACATACATC*TAGTACATGTCTAGTC
AGTATCTAGTGATTA*T*CCCCC/
(SEQ ID NO: 92)
CCCCC*A*TAATCACTAGATACTGACTAGACATGTACTAGATGTATGTCTAGATA
ATCACTAGATACTGAC*T*CCCCC
(14):
(SEQ ID NO: 55)
CCCCC*A*GTCAGTATCTAGTGATTATCTAGACATACATCTAGTACATGTCTAGTCA
GTATCTAGTGATTA*T*CCCCC/
(SEQ ID NO: 92)
CCCCC*A*TAATCACTAGATACTGACTAGACATGTACTAGATGTATGTCTAGATA
ATCACTAGATACTGAC*T*CCCCC
(15):
(SEQ ID NO: 105)
CCCCC*AGTCAGTATCTAGTGATTATCTAGACATACATCTAGTACATGTCTAGTCAG
TATCTAGTGATTAT*CCCCC/
(SEQ ID NO: 106)
CCCCC*ATAATCACTAGATACTGACTAGACATGTACTAGATGTATGTCTAGATA
ATCACTAGATACTGACT*CCCCC
(16):
(SEQ ID NO: 107)
CCCCCA*GTCAGTATCTAGTGATTATCTAGACATACATCTAGTACATGTCTAGTCAG
TATCTAGTGATTA*TCCCCC/
(SEQ ID NO: 108)
CCCCCA*TAATCACTAGATACTGACTAGACATGTACTAGATGTATGTCTAGATA
ATCACTAGATACTGAC*TCCCCC

From this experiment, we conclude that adding boundary PT linkages on either strand increases SIDSP activation, and that having boundary PT linkages on both strands ((14), (15), (16)) gives rise to the most potent ligands, stimulating IFNB production via the SIDSP at levels 25-100×above the same substrate without PT linkages ((1)). Also, having single PT linkages at the boundaries ((15), (16)) is just as potent as having two PT linkages at each boundary ((14)).

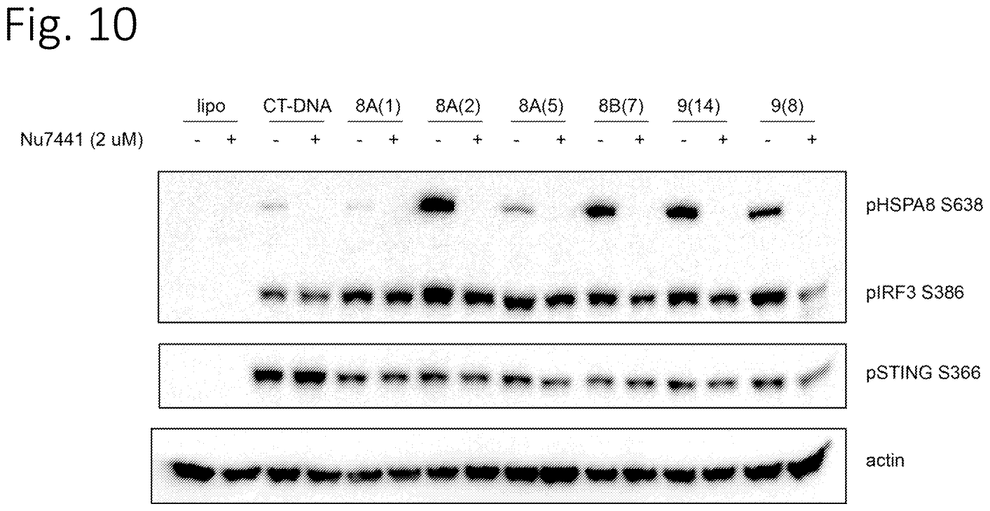

FIG. 10 shows that the potent SIDSP ligands from FIGS. 8 and 9 also activate cGAS/STING. Duplex DNAs were annealed and transfected into tert-immortalized human foreskin fibroblasts at 4 ug/mL final concentration using lipofectamine reagent following a 1 hour pretreatment with either DMSO (1:2500 dilution) or the DNA-PK inhibitor Nu7441 at 2 uM, After 6 hours, cells were harvested in RIPA buffer and lysates were run through gel electrophoresis. Proteins were transferred to nitrocellulose membranes, blocked with 5% BSA in TBST, washed, and blotted with antibodies to recognize the following phosphorylation events: Phosphorylated HSPA8 (Ser638) is a readout for DNA-PK/SIDSP activation. Phosphorylated IRF3 (Ser386) is a readout for both cGAS/STING and DNA-PK/SIDSP activation. Phosphorylated STING (Ser366) is a readout for cGAS/STING activation.

### Substrates Tested:

8A(2):
(SEQ ID NO: 268)
CCCCCAGTCAG*T*A*T*C*T*A*G*T*G*A*T*T*A*T*C*T*A*G*A*
C*A*T*A*C*A**C*T*A*G*T*A*C*A*T*G*T*C*T*A*G*T*C*A*
G*T*A*T*C*T*A*G*T*GATTATCCCCC
(SEQ ID NO: 22)
/CCCCCATAATCACTAGATACTGACTAGACATGTACTAGATGTATGTCTA
GATAATCACTAGATACTGACTCCCCC
8A(1):
(SEQ ID NO: 21)
CCCCCAGTCAGTATCTAGTGATTATCTAGACATACATCTAGTACATGTCT
AGTCAGTATCTAGTGATTATCCCCC
(SEQ ID NO: 22)
/CCCCCATAATCACTAGATACTGACTAGACATGTACTAGATGTATGTCTA
GATAATCACTAGATACTGACTCCCCC
8A(5):
(SEQ ID NO: 51)
CCCCCAGTCAGTATCTAGTGATTATCTAGACATACA*T*C*T*A*GTACA
TGTCTAGTCAGTATCTAGTGATTATCCCCC
(SEQ ID NO: 22)
/CCCCCATAATCACTAGATACTGACTAGACATGTACTAGATGTATGTCTA
GATAATCACTAGATACTGACTCCCCC
8B(7):
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 22)
/CCCCCATAATCACTAGATACTGACTAGACATGTACTAGATGTATGTCTA
GATAATCACTAGATACTGACTCCCCC
9(14):
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC
9(8):
(SEQ ID NO: 51)
CCCCCAGTCAGTATCTAGTGATTATCTAGACATACA*T*C*T*A*GTACA
TGTCTAGTCAGTATCTAGTGATTATCCCCC
(SEQ ID NO: 92)
/CCCCC*A*TAATCACTAGATACTGACTAGACATGTACTAGATGTATGTC
TAGATAATCACTAGATACTGAC*T*CCCCC

From this experiment, we conclude that the potent SIDSP ligands from FIGS. 8 and 9 also activate cGAS/STING, as evidenced by pSTING S366 signal. Also, as expected, the pHSPA8 S638 signal in each case is completely dependent on DNA-PK, as evidence by complete loss with inhibitor pretreatment.

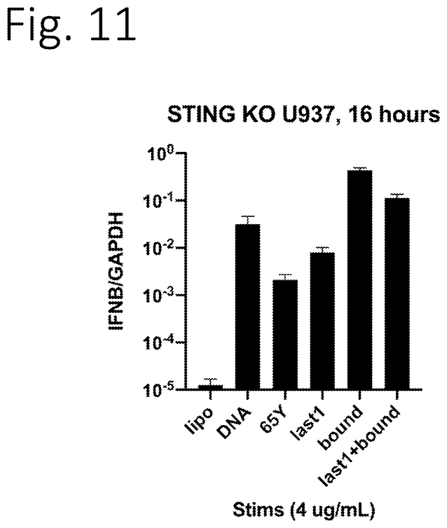

FIG. 11 shows that combined PT linkages at both the termini and single-stranded/double-stranded boundary regions does not activate the SIDSP to a greater extent than with PT linkages at the boundary regions alone. Duplex oligos were designed as Y-form (Y) 65 bp substrates with 5 bp C-containing overhangs, containing either phosphodiester linkages or phosphorothioate (PT) linkages. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

65Y:
(SEQ ID NO: 21)
CCCCCAGTCAGTATCTAGTGATTATCTAGACATACATCTAGTACATGTCT
AGTCAGTATCTAGTGATTATCCCCC
(SEQ ID NO: 22)
/CCCCCATAATCACTAGATACTGACTAGACATGTACTAGATGTATGTCTA
GATAATCACTAGATACTGACTCCCCC
last1:
(SEQ ID NO: 53)
C*CCCCAGTCAGTATCTAGTGATTATCTAGACATACATCTAGTACATGTC
TAGTCAGTATCTAGTGATTATCCCC*C
(SEQ ID NO: 22)
/CCCCCATAATCACTAGATACTGACTAGACATGTACTAGATGTATGTCTA
GATAATCACTAGATACTGACTCCCCC
bound:
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC
last1+bound:
(SEQ ID NO: 125)
C*CCCC*A*GTCAGTATCTAGTGATTATCTAGACATACATCTAGTACATG
TCTAGTCAGTATCTAGTGATTA*T*CCCC*C
(SEQ ID NO: 126)
/C*CCCC*A*TAATCACTAGATACTGACTAGACATGTACTAGATGTATGT
CTAGATAATCACTAGATACTGAC*T*CCCC*C

From this experiment, we conclude that the addition of a terminal PT linkage in combination with boundary PT linkages does not enhance SIDSP activation over boundary PT linkages alone.

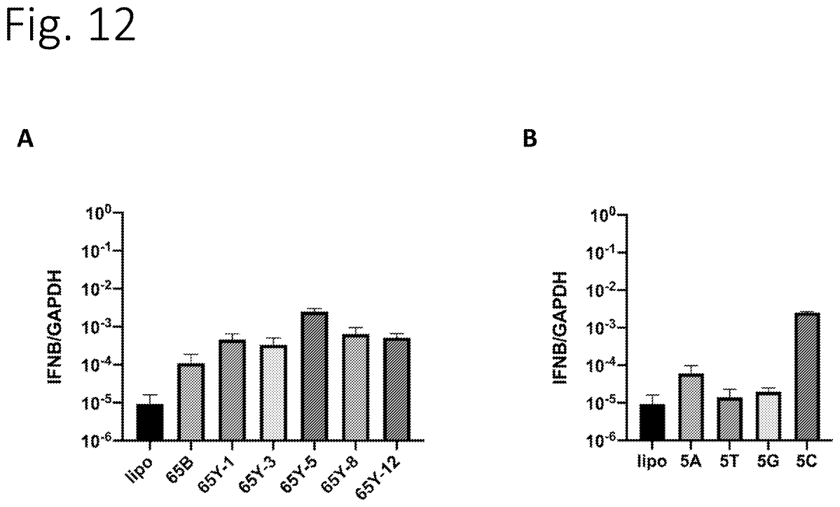

FIG. 12 shows the effect of overhang length and content on the SIDSP-induced interferon response in the context of 65 base pair oligo duplex ligands. A) Duplex oligos were designed as Y-form 65 bp substrates with 5′ and 3′ C-containing overhangs ranging from 1-12 bp in length. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

65B:
(SEQ ID NO: 127)
AGTCAGTATCTAGTGATTATCTAGACATACATCTAGTACATGTCTAGTCA
GTATCTAGTGATTAT
(SEQ ID NO: 128)
/ATAATCACTAGATACTGACTAGACATGTACTAGATGTATGTCTAGATAA
TCACTAGATACTGACT
65Y-1:
(SEQ ID NO: 129)
CAGTCAGTATCTAGTGATTATCTAGACATACATCTAGTACATGTCTAGTC
AGTATCTAGTGATTATC
(SEQ ID NO: 130)
/CATAATCACTAGATACTGACTAGACATGTACTAGATGTATGTCTAGATA
ATCACTAGATACTGACTC
65Y-3:
(SEQ ID NO: 131)
CCCAGTCAGTATCTAGTGATTATCTAGACATACATCTAGTACATGTCTAG
TCAGTATCTAGTGATTATCCC
(SEQ ID NO: 132)
/CCCATAATCACTAGATACTGACTAGACATGTACTAGATGTATGTCTAGA
TAATCACTAGATACTGACTCCC
65Y-5:
(SEQ ID NO: 21)
CCCCCAGTCAGTATCTAGTGATTATCTAGACATACATCTAGTACATGTCT
AGTCAGTATCTAGTGATTATCCCCC
(SEQ ID NO: 22)
/CCCCCATAATCACTAGATACTGACTAGACATGTACTAGATGTATGTCTA
GATAATCACTAGATACTGACTCCCCC
65Y-8:
(SEQ ID NO: 135)
CCCCCCCCAGTCAGTATCTAGTGATTATCTAGACATACATCTAGTACATG
TCTAGTCAGTATCTAGTGATTATCCCCCCCC
(SEQ ID NO: 136)
/CCCCCCCCATAATCACTAGATACTGACTAGACATGTACTAGATGTATGT
CTAGATAATCACTAGATACTGACTCCCCCCCC
65Y-12:
(SEQ ID NO: 137)
CCCCCCCCCCCCAGTCAGTATCTAGTGATTATCTAGACATACATCTAGTA
CATGTCTAGTCAGTATCTAGTGATTATCCCCCCCCCCCC
(SEQ ID NO: 138)
/CCCCCCCCCCCCATAATCACTAGATACTGACTAGACATGTACTAGATGT
ATGTCTAGATAATCACTAGATACTGACTCCCCCCCCCCCC

From this experiment, we conclude that overhangs of any length increase IFNB production above the corresponding blunt oligo duplex, with 5 bp overhangs being optimal.

B) Duplex oligos were designed as Y-form 65 bp substrates with 5 bp overhangs containing As, Ts, Gs, or Cs. Duplex DNAs were annealed and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

5T:
(SEQ ID NO: 274)
TTTTTAGTCAGTATCTAGTGATTATCTAGACATACATCTAGTACATGTCT
AGTCAGTATCTAGTGATTATTTTTT
(SEQ ID NO: 275)
/TTTTTATAATCACTAGATACTGACTAGACATGTACTAGATGTATGTCTA
GATAATCACTAGATACTGACTTTTTT
5G:
(SEQ ID NO: 276)
GGGGGAGTCAGTATCTAGTGATTATCTAGACATACATCTAGTACATGTCT
AGTCAGTATCTAGTGATTATGGGGG
(SEQ ID NO: 277)
/GGGGGATAATCACTAGATACTGACTAGACATGTACTAGATGTATGTCTA
GATAATCACTAGATACTGACTGGGGG
5A:
(SEQ ID NO: 139)
AAAAAAGTCAGTATCTAGTGATTATCTAGACATACATCTAGTACATGTCT
AGTCAGTATCTAGTGATTATAAAAA
(SEQ ID NO: 140)
/AAAAAATAATCACTAGATACTGACTAGACATGTACTAGATGTATGTCTA
GATAATCACTAGATACTGACTAAAAA
5C:
(SEQ ID NO: 21)
CCCCCAGTCAGTATCTAGTGATTATCTAGACATACATCTAGTACATGTCT
AGTCAGTATCTAGTGATTATCCCCC
(SEQ ID NO: 22)
/CCCCCATAATCACTAGATACTGACTAGACATGTACTAGATGTATGTCTA
GATAATCACTAGATACTGACTCCCCC

From this experiment, we confirm that cytosine-containing overhangs on 65 bp duplexes activate the SIDSP most potently, with some activation resulting from 65 bp duplexes with adenine-containing overhangs.

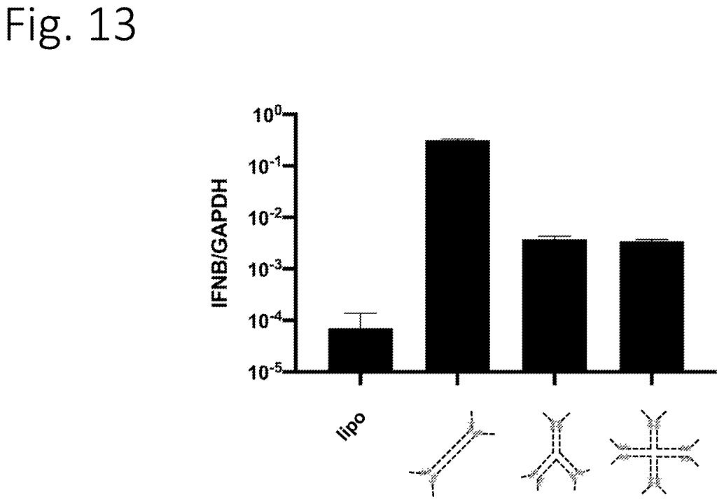

FIG. 13 shows the effect of increasing the number of Y branches in a single ligand molecule on SIDSP activation. A) Oligos were designed as Y-form 65 bp substrates with 5 bp C-containing overhangs. DNAs were annealed either as a duplex (2 strands), triplex (3 strands), or quadruplex (4 strands), and transfected into STING deficient U937 cells at 4 ug/mL final concentration using lipofectamine reagent. After 16 hours, cells were harvested in Trizol and RNA was purified using the Zymo Directrol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

Duplex:
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC
Triplex:
(SEQ ID NO: 145)
CCCCC*A*GTCAGTATCTAGTGATTATCTAGACATACATCTAGTACATGT
CTAGTCAGTATGTAGTGATTA*T*CCCCC
(SEQ ID NO: 146)
/CCCCC*A*TAATCACTACATACTGACTAGACATGTACTAGACTCTGTTT
CAAGAGAATAACTTGAGATTCA*A*CCCCC
(SEQ ID NO: 147)
/CCCCC*T*TGAATCTCAAGTTATTCTCTTGAAACAGAGTATGTATGTCT
AGATAATCACTAGATACTGACT*T*CCCCC
Quadruplex:
(SEQ ID NO: 145)
CCCCC*A*GTCAGTATCTAGTGATTATCTAGACATACATCTAGTACATGT
CTAGTCAGTATGTAGTGATTA*T*CCCCC
(SEQ ID NO: 149)
/CCCCC*A*TGTGAGAATGTACTGTTGTTCAAGTCAAACTTATGTATGTC
TAGATAATCACTAGATACTGAC*T*CCCCC
(SEQ ID NO: 146)
/CCCCC*A*TAATCACTACATACTGACTAGACATGTACTAGACTCTGTTT
CAAGAGAATAACTTGAGATTCA*A*CCCCC
(SEQ ID NO: 151)
/CCCCC*T*TGAATCTCAAGTTATTCTCTTGAAACAGAGTAAGTTTGACT
TGAACAACAGTACATTCTCACA*T*CCCCC

From this experiment, we conclude that 3- or 4-pronged Y-form substrates do not increase SIDSP activation over that of traditional Y-form duplexes.

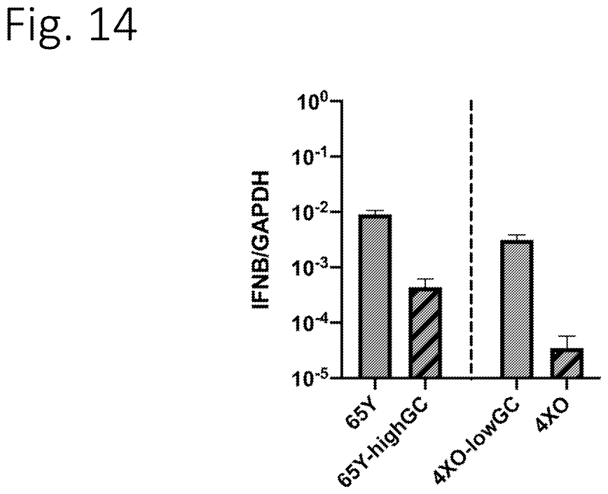

FIG. 14 shows the effect of GC content in the double-stranded portion of the SIDSP agonist. Oligos were designed as Y-form 65 bp substrates with 5 bp C-containing overhangs. Starting with two distinct sequences (“65Y” and “4XO”), minimal changes were made in order to drastically increase or decrease the GC content of the double-stranded portion. DNAs were annealed as a duplex and transfected into STING deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

4XO:
(SEQ ID NO: 278)
CCCCCTGGGTGAACCAGCAGGTGGGCAAAGATGCAGTCCTAGCAATGTAA
TCGTCAAGCTTTATGCCGTTCCCCC
(GC content of ds region: 49%)
(SEQ ID NO: 279)
/CCCCCAACGGCATAAAGCTTGACGATTACATTGCTAGGACTGCATCTTT
GCCCACCTGCTGGTTCACCCACCCCC
65Y:
(SEQ ID NO: 21)
CCCCCAGTCAGTATCTAGTGATTATCTAGACATACATCTAGTACATGTCT
AGTCAGTATCTAGTGATTATCCCCC
(GC content of ds region: 32%)
(SEQ ID NO: 22)
/CCCCCATAATCACTAGATACTGACTAGACATGTACTAGATGTATGTCTA
GATAATCACTAGATACTGACTCCCCC
65Y-highGC:
(SEQ ID NO: 154)
CCCCCAGTCAGCCTCCAGTGAGCATCTAGACATGCATCTAGTGCCTGTCC
AGCCAGCCTCTGGTGATTATCCCCC
(GC content of ds region: 52%)
(SEQ ID NO: 155)
/CCCCCATAATCACCAGAGGCTGGCTGGACAGGCACTAGATGCATGTCTA
GATGCTCACTGGAGGCTGACTCCCCC
4XO-lowGC:
(SEQ ID NO: 156)
CCCCCTGAATGAACAATCATCTATAGACAGATACAGTATTATCAATGTAA
TGTACAATCTTTATGAGCTTCCCCC
(GC content of ds region: 28%
(SEQ ID NO: 157)
/CCCCCAAGCTCATAAAGATTGTACATTACATTGATAATACTGTATCTGT
CTATAGATGATTGTTCATTCACCCCC

From this experiment, we conclude that low GC content in the double-stranded region of our agonist designs more potently stimulates the SIDSP, whereas high GC content decreases their potency.

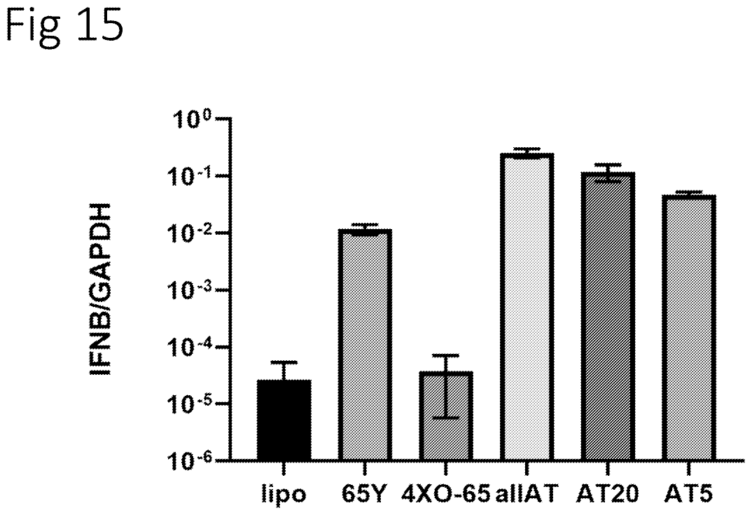

FIG. 15 further shows the effect of GC content in the double-stranded portion of the SIDSP agonist. Oligos were designed as Y-form 65 bp substrates with 5 bp C-containing overhangs. DNAs were annealed as a duplex and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

4XO-65:
(SEQ ID NO: 278)
CCCCCTGGGTGAACCAGCAGGTGGGCAAAGATGCAGTCCTAGCAATGTAA
TCGTCAAGCTTTATGCCGTTCCCCC
(GC content of ds region: 49%)
(SEQ ID NO: 279)
/CCCCCAACGGCATAAAGCTTGACGATTACATTGCTAGGACTGCATCTTT
GCCCACCTGCTGGTTCACCCACCCCC
allAT:
(SEQ ID NO: 160)
CCCCCATATATATATATATATATATATATATATATATATATATATATATA
TATATATATATATATATATACCCCC
(GC content of ds region: 0%)
(SEQ ID NO: 161)
/CCCCCTATATATATATATATATATATATATATATATATATATATATATA
TATATATATATATATATATATCCCCC
AT20:
(SEQ ID NO: 162)
CCCCCATATATATATATATATATATACATATATATATATATATATATAGA
TATATATATATATATATATACCCCC
(GC content of ds region: 3%)
(SEQ ID NO: 163)
/CCCCCTATATATATATATATATATATCTATATATATATATATATATATG
TATATATATATATATATATATCCCCC
AT5:
(SEQ ID NO: 164)
CCCCCATATACTATATGATATAGTATATCATATACTATATGATATAGTAT
ATCATATACTATATGATATACCCCC
(GC content of ds region: 15%)
(SEQ ID NO: 165)
/CCCCCTATATCATATAGTATATGATATACTATATCATATAGTATATGAT
ATACTATATCATATAGTATATCCCCC

From this experiment, we conclude that GC content as low as 0% in the double-stranded region of our agonist designs more potently stimulates the SIDSP.

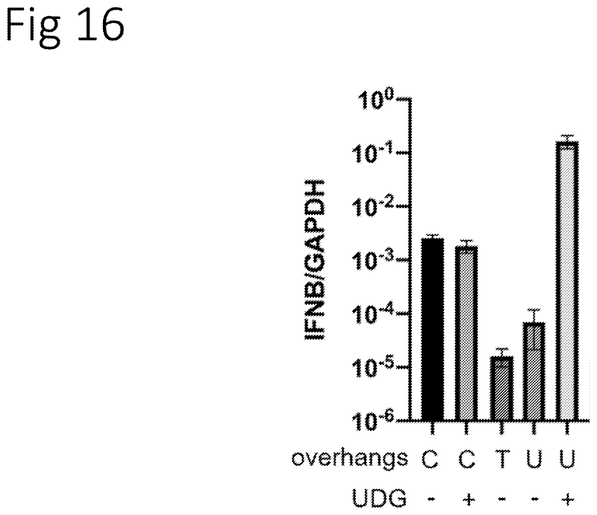

FIG. 16 shows the effect of abasic sites in the overhangs of the prototype SIDSP agonists. Oligos were designed as Y-form 65 bp substrates with 5 bp overhangs of either deoxycytosines (C), deoxythymines (T), or deoxyuracils (U). DNAs were annealed as a duplex, and then treated either with mock buffer or with Uracil DNA glycosylase (UDG) in UDG buffer for 10 minutes at 37 C, followed by a heat quench at 95 C for 10 minutes. Uracil DNA glycosylase inhibitor (UGI) was then added in excess to completely stop any further reaction. UDG treatment of DNAs containing deoxyuracils will generate abasic sites. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested: (Names Denote Overhang Content)

“T”:
(SEQ ID NO: 274)
TTTTTAGTCAGTATCTAGTGATTATCTAGACATACATCTAGTACATGTCT
AGTCAGTATCTAGTGATTATTTTTT
(SEQ ID NO: 275)
/TTTTTATAATCACTAGATACTGACTAGACATGTACTAGATGTATGTCTA
GATAATCACTAGATACTGACTTTTTT
“U”:
(SEQ ID NO: 280)
UUUUUAGTCAGTATCTAGTGATTATCTAGACATACATCTAGTACATGTCT
AGTCAGTATCTAGTGATTATUUUUU
(SEQ ID NO: 281)
/UUUUUATAATCACTAGATACTGACTAGACATGTACTAGATGTATGTCTA
GATAATCACTAGATACTGACTUUUUU
“C”:
(SEQ ID NO: 21)
CCCCCAGTCAGTATCTAGTGATTATCTAGACATACATCTAGTACATGTCT
AGTCAGTATCTAGTGATTATCCCCC
(SEQ ID NO: 22)
/CCCCCATAATCACTAGATACTGACTAGACATGTACTAGATGTATGTCTA
GATAATCACTAGATACTGACTCCCCC
″U″ (+UDG):
(SEQ ID NO: 168)
[abasic][abasic][abasic][abasic][abasic]AGTCAGTATC
TAGTGATTATCTAGACATACATCTAGTACATGTCTAGTCAGTATCTAGTG
ATTAT[abasic][abasic][abasic][abasic][abasic]
(SEQ ID NO: 169)
/[abasic][abasic][abasic][abasic][abasic]ATAATCACT
AGATACTGACTAGACATGTACTAGATGTATGTCTAGATAATCACTAGATA
CTGACT[abasic][abasic][abasic][abasic][abasic]

From this experiment, we conclude that abasic sites in the overhangs, generated from UDG treatment of deoxyuracil-containing DNA, effect highly stimulatory SIDSP agonists. However, deoxyuracil in the overhangs alone does not activate the SIDSP.

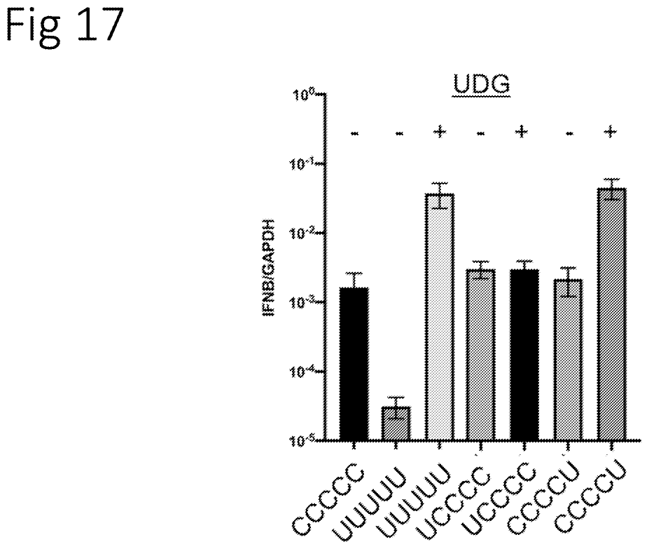

FIG. 17 shows the effect of specific abasic sites in the overhangs of the prototype SIDSP agonists. Oligos were designed as Y-form 65 bp substrates with 5 bp overhangs containing a mixture of deoxycytosines (C) and deoxyuracils (U). DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested: (Names Denote Overhang Content)

“UUUUU”:
(SEQ ID NO: 280)
UUUUUAGTCAGTATCTAGTGATTATCTAGACATACATCTAGTACATGTCT
AGTCAGTATCTAGTGATTATUUUUU
(SEQ ID NO: 281)
/UUUUUATAATCACTAGATACTGACTAGACATGTACTAGATGTATGTCTA
GATAATCACTAGATACTGACTUUUUU
“CCCCC”:
(SEQ ID NO: 21)
CCCCCAGTCAGTATCTAGTGATTATCTAGACATACATCTAGTACATGTCT
AGTCAGTATCTAGTGATTATCCCCC
(SEQ ID NO: 22)
/CCCCCATAATCACTAGATACTGACTAGACATGTACTAGATGTATGTCTA
GATAATCACTAGATACTGACTCCCCC
“UUUUU”: (+UDG):
(SEQ ID NO: 168)
[abasic][abasic][abasic][abasic][abasic]AGTCAGTATC
TAGTGATTATCTAGACATACATCTAGTACATGTCTAGTCAGTATCTAGTG
ATTAT[abasic][abasic][abasic][abasic][abasic]
(SEQ ID NO: 169)
/[abasic][abasic][abasic][abasic][abasic]ATAATCACT
AGATACTGACTAGACATGTACTAGATGTATGTCTAGATAATCACTAGATA
CTGACT[abasic][abasic][abasic][abasic][abasic]
“UCCCC”:
(SEQ ID NO: 174)
UCCCCAGTCAGTATCTAGTGATTATCTAGACATACATCTAGTACATGTCT
AGTCAGTATCTAGTGATTATCCCCU
(SEQ ID NO: 175)
/UCCCCATAATCACTAGATACTGACTAGACATGTACTAGATGTATGTCTA
GATAATCACTAGATACTGACTCCCCU
“UCCCC” (+UDG):
(SEQ ID NO: 176)
[abasic]CCCCAGTCAGTATCTAGTGATTATCTAGACATACATCTAGTA
CATGTCTAGTCAGTATCTAGTGATTATCCCC[abasic]
(SEQ ID NO: 177)
/[abasic]CCCCATAATCACTAGATACTGACTAGACATGTACTAGATGT
ATGTCTAGATAATCACTAGATACTGACTCCCC[abasic]
“CCCCU”:
(SEQ ID NO: 178)
CCCCUAGTCAGTATCTAGTGATTATCTAGACATACATCTAGTACATGTCT
AGTCAGTATCTAGTGATTATUCCCC
(SEQ ID NO: 179)
/CCCCUATAATCACTAGATACTGACTAGACATGTACTAGATGTATGTCTA
GATAATCACTAGATACTGACTUCCCC
“CCCCU” (+UDG):
(SEQ ID NO: 180)
CCCC[abasic]AGTCAGTATCTAGTGATTATCTAGACATACATCTAGTA
CATGTCTAGTCAGTATCTAGTGATTAT[abasic]CCCC
(SEQ ID NO: 181)
/CCCC[abasic]ATAATCACTAGATACTGACTAGACATGTACTAGATGT
ATGTCTAGATAATCACTAGATACTGACT[abasic]CCCC

From this experiment, we conclude that abasic sites in the overhangs, generated from UDG treatment of deoxyuracil-containing DNA, effect highly stimulatory SIDSP agonists. Specifically, abasic sites at the single-stranded/double-stranded boundary region effect the most stimulatory activity.

FIG. 18 shows the effect of natural versus synthetic abasic sites on the potency of prototype SIDSP agonists. Oligos were designed as Y-form 65 bp substrates with 5 bp overhangs containing either a mixture of deoxycytosines (C) and deoxyuracils (U)-leftmost structure in FIG. 18B, denoting 1′ OH— or a mixture of deoxycytosines (C) and “dSpacer” modifications from IDT-rightmost structure in FIG. 18B, pink box denoting lack of 1′ OH. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested: (Names Denote Overhang Content)

“CCCCU” (+UDG):
(SEQ ID NO: 180)
CCCC[abasic]AGTCAGTATCTAGTGATTATCTAGACATACATCTAGTA
CATGTCTAGTCAGTATCTAGTGATTAT[abasic]CCCC
(SEQ ID NO: 181)
/CCCC[abasic]ATAATCACTAGATACTGACTAGACATGTACTAGATGT
ATGTCTAGATAATCACTAGATACTGACT[abasic]CCCC
“CCCC[ ]”:
(SEQ ID NO: 184)
CCCC[dSpacer]AGTCAGTATCTAGTGATTATCTAGACATACATCTAGT
ACATGTCTAGTCAGTATCTAGTGATTAT[dSpacer]CCCC
(SEQ ID NO: 185)
/CCCC[dSpacer]ATAATCACTAGATACTGACTAGACATGTACTAGATG
TATGTCTAGATAATCACTAGATACTGACT[dSpacer]CCCC

From this experiment, we conclude that “natural” abasic sites, generated by Uracil DNA glycosylase (UDG) treatment of deoxyuracils in DNA, are potent agonists of the SIDSP. However, “synthetic” abasic sites, lacking the 1′ OH on the sugar ring, are not potent SIDSP agonists.

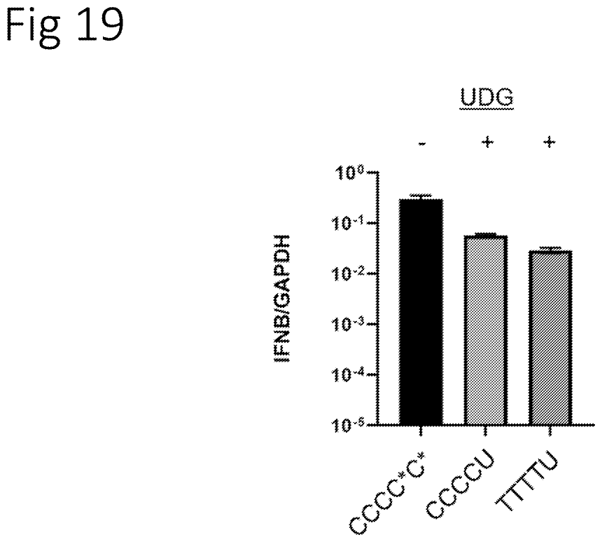

FIG. 19 shows the effect of abasic sites in otherwise inert prototype SIDSP agonists. Oligos were designed as Y-form 65 bp substrates with 5 bp overhangs containing either deoxycytosines (C), deoxythymines (T), or deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested: (Names Denote Overhang Content)

“CCCC*C*”:
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC (asterisks denote
locations of phosphorothioate linkages)
“CCCCU” (+UDG) :
(SEQ ID NO: 180)
CCCC[abasic]AGTCAGTATCTAGTGATTATCTAGACATACATCTAGTA
CATGTCTAGTCAGTATCTAGTGATTAT[abasic]CCCC
(SEQ ID NO: 181)
/CCCC[abasic]ATAATCACTAGATACTGACTAGACATGTACTAGATGT
ATGTCTAGATAATCACTAGATACTGACT[abasic]CCCC
“TTTTU” (+UDG) :
(SEQ ID NO: 190)
TTTT[abasic]AGTCAGTATCTAGTGATTATCTAGACATACATCTAGTA
CATGTCTAGTCAGTATCTAGTGATTAT[abasic]TTTT
(SEQ ID NO: 191)
/TTTT[abasic]ATAATCACTAGATACTGACTAGACATGTACTAGATGT
ATGTCTAGATAATCACTAGATACTGACT[abasic]TTTT

From this experiment, we conclude that the presence of abasic sites (specifically, those at the single-stranded/double-stranded boundary position) is sufficient to make previously inert 65 bp Y-overhang DNA (e.g. ST overhangs, FIG. 12B) into potent agonists.

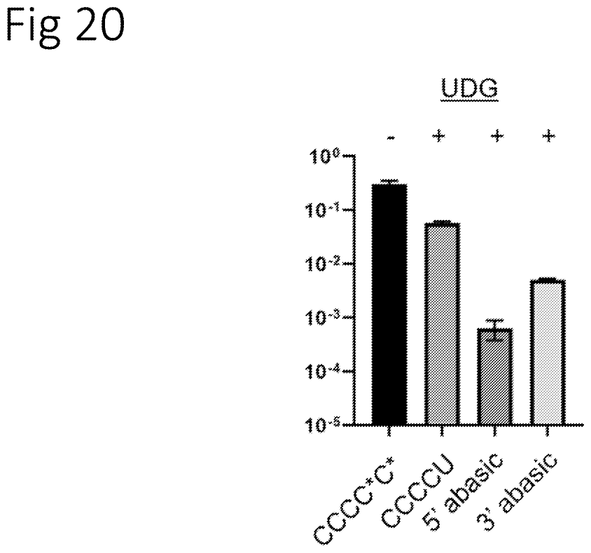

FIG. 20 shows the effect of abasic sites in specific locations in the overhangs of prototype SIDSP agonists. Oligos were designed as Y-form 65 bp substrates with 5 bp overhangs containing a mixture of deoxycytosines (C) and deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction, DNAs were then reannealed and transfected into STING-deficient U937 cells at 4 ug/mL final concentration using lipofectaniine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

“CCCC*C*”:
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC (asterisks denote
locations of phosphorothioate linkages)
“CCCCU” (+UDG):
(SEQ ID NO: 180)
CCCC[abasic]AGTCAGTATCTAGTGATTATCTAGACATACATCTAGTA
CATGTCTAGTCAGTATCTAGTGATTAT[abasic]CCCC
(SEQ ID NO: 181)
/CCCC[abasic]ATAATCACTAGATACTGACTAGACATGTACTAGATGT
ATGTCTAGATAATCACTAGATACTGACT[abasic]CCCC
5′ abasic (+UDG):
(SEQ ID NO: 196)
CCCC[abasic]AGTCAGTATCTAGTGATTATCTAGACATACATCTAGTA
CATGTCTAGTCAGTATCTAGTGATTATCCCCC
(SEQ ID NO: 197)
/CCCC[abasic]ATAATCACTAGATACTGACTAGACATGTACTAGATGT
ATGTCTAGATAATCACTAGATACTGACTCCCCC
3′ abasic (+UDG):
(SEQ ID NO: 198)
CCCCCAGTCAGTATCTAGTGATTATCTAGACATACATCTAGTACATGTCT
AGTCAGTATCTAGTGATTAT[abasic]CCCC
(SEQ ID NO: 199)
/CCCCCATAATCACTAGATACTGACTAGACATGTACTAGATGTATGTCTA
GATAATCACTAGATACTGACT[abasic]CCCC

From this experiment, we conclude having one abasic site in each strand is intermediately potent, but having two abasic sites per strand (one in each overhang—CCCCU) is the most potent.

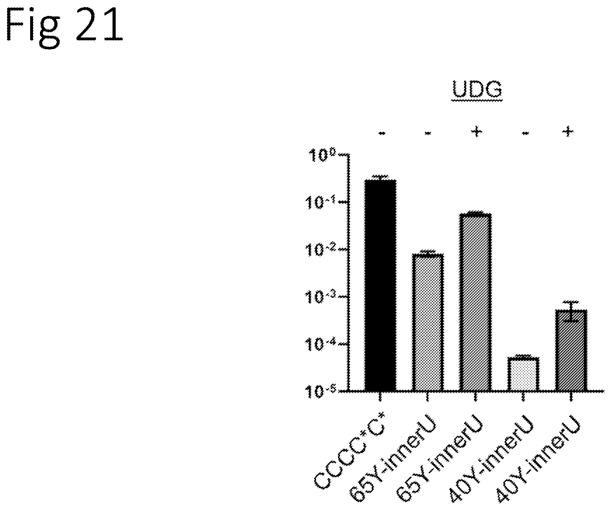

FIG. 21 shows the effect of abasic sites in the overhangs of shorter prototype SIDSP agonists. Oligos were designed as Y-form substrates where the double-stranded region was either 65 bp or 40 bp. All oligos contained 5 bp overhangs with a mixture of deoxycytosines (C) and deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

40Y-innerU:
(SEQ ID NO: 282)
CCCCUAGTCAGTATCTAGTGATTATAGTCAGTATCTAGTGATTATUCCCC
(SEQ ID NO: 283)
/CCCCUATAATCACTAGATACTGACTATAATCACTAGATACTGACTUCCC
C
“CCCC*C*”:
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC (asterisks denote
locations of phosphorothioate linkages)
65Y-innerU:
(SEQ ID NO: 178)
CCCCUAGTCAGTATCTAGTGATTATCTAGACATACATCTAGTACATGTCT
AGTCAGTATCTAGTGATTATUCCCC
(SEQ ID NO: 179)
/CCCCUATAATCACTAGATACTGACTAGACATGTACTAGATGTATGTCTA
GATAATCACTAGATACTGACTUCCCC
65Y-innerU (+UDG):
(SEQ ID NO: 180)
CCCC[abasic]AGTCAGTATCTAGTGATTATCTAGACATACATCTAGTA
CATGTCTAGTCAGTATCTAGTGATTAT[abasic]CCCC
(SEQ ID NO: 181)
/CCCC[abasic]ATAATCACTAGATACTGACTAGACATGTACTAGATGT
ATGTCTAGATAATCACTAGATACTGACT[abasic]CCCC
40Y-innerU (+UDG):
(SEQ ID NO: 206)
CCCC[abasic]AGTCAGTATCTAGTGATTATAGTCAGTATCTAGTGATT
AT[abasic]CCCC
(SEQ ID NO: 207)
/CCCC[abasic]ATAATCACTAGATACTGACTATAATCACTAGATACTG
ACT[abasic]CCCC

From this experiment, we conclude that having abasic sites in the overhangs of shorter (>=35 bp) SIDSP agonists increase the potency of these agonists to generate an interferon response.

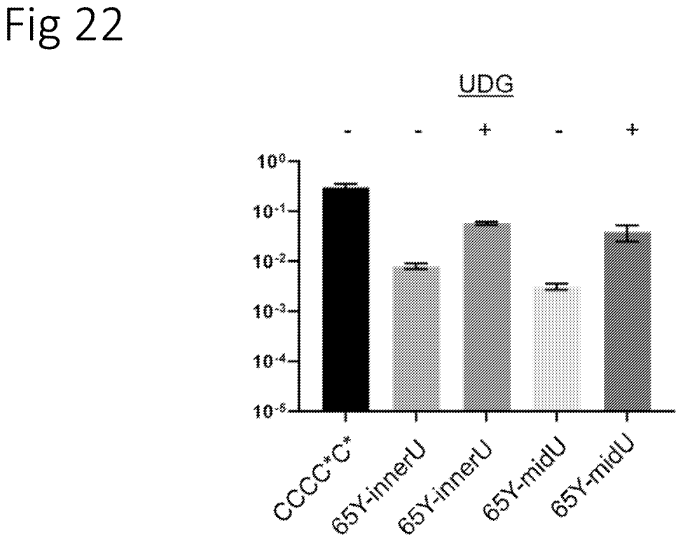

FIG. 22 shows the effect of abasic sites at specific positions in the overhangs of prototype SIDSP agonists. Oligos were designed as 65 bp Y-form substrates containing 5 bp overhangs with a mixture of deoxycytosines (C) and deoxyuracils (U) as denoted in the sequences below. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

“CCCC*C*”:
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC (Note: asterisks
denote locations of phosphorothioate linkages)
65Y-innerU:
(SEQ ID NO: 178)
CCCCUAGTCAGTATCTAGTGATTATCTAGACATACATCTAGTACATGTCT
AGTCAGTATCTAGTGATTATUCCCC
(SEQ ID NO: 179)
/CCCCUATAATCACTAGATACTGACTAGACATGTACTAGATGTATGTCTA
GATAATCACTAGATACTGACTUCCCC
65Y-innerU (+UDG):
(SEQ ID NO: 180)
CCCC[abasic]AGTCAGTATCTAGTGATTATCTAGACATACATCTAGTA
CATGTCTAGTCAGTATCTAGTGATTAT[abasic]CCCC
(SEQ ID NO: 181)
/CCCC[abasic]ATAATCACTAGATACTGACTAGACATGTACTAGATGT
ATGTCTAGATAATCACTAGATACTGACT[abasic]CCCC
65Y-midU:
(SEQ ID NO: 214)
CCUCCAGTCAGTATCTAGTGATTATCTAGACATACATCTAGTACATGTCT
AGTCAGTATCTAGTGATTATCCUCC
(SEQ ID NO: 215)
/CCUCCATAATCACTAGATACTGACTAGACATGTACTAGATGTATGTCTA
GATAATCACTAGATACTGACTCCUCC
65Y-midU (+UDG):
(SEQ ID NO: 216)
CC[abasic]CCAGTCAGTATCTAGTGATTATCTAGACATACATCTAGTA
CATGTCTAGTCAGTATCTAGTGATTATCC[abasic]CC
(SEQ ID NO: 217)
/CC[abasic]CCATAATCACTAGATACTGACTAGACATGTACTAGATGT
ATGTCTAGATAATCACTAGATACTGACTCC[abasic]CC

From this experiment, we concluded that abasic sites either at the single-stranded/double-stranded boundary position (“inner”) or in the middle of the single-stranded overhang effect potent SIDSP agonism.

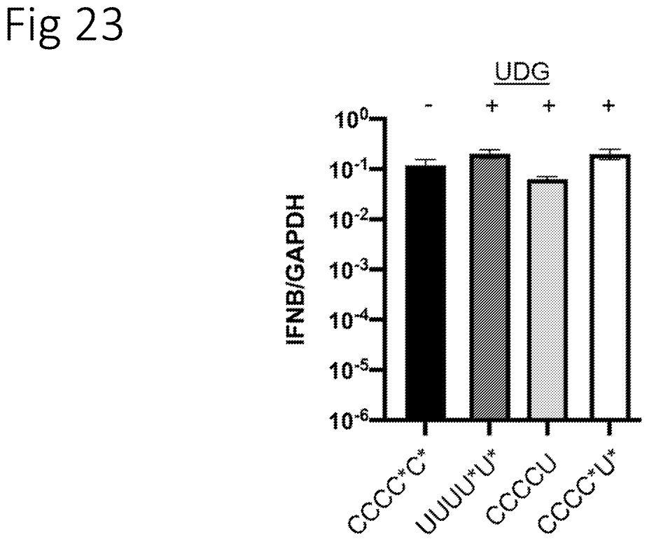

FIG. 23 shows the effect of combining phosphorothioate linkages and abasic sites at specific positions in the overhangs of prototype SIDSP agonists. Oligos were designed as 65 bp Y-form substrates containing 5 bp overhangs with a mixture of deoxycytosines (C) and deoxyuracils (U) as denoted in the sequences below. Asterisks denote the locations of phosphorothioate linkages. DNAs were annealed as a duplex, and then treated either with mock buffer or with UDG in UDG buffer for 10 minutes at 37 C, followed by a heat quench at 95 C for 10 minutes. Uracil DNA glycosylase inhibitor (UGI) was then added in excess to completely stop any further reaction. DNAs were then reannealed and transfected into STING-deficient U937 cells at 4 ug/mL final concentration using lipofectamine reagent. After 16 hours, cells were harvested in Trizol and RNA was purified using the Zymo Directzol™ RNA purification kit. cDNA was generated via reverse transcription using the RNA to cDNA EcoDry™ Premix (Takara Biosciences), and quantitative PCR (qPCR) was performed using primers to amplify the IFNB and GAPDH loci. IFNB transcript levels were normalized to those of GAPDH for each condition. Experiment was performed in biological triplicate and technical duplicate.

### Substrates Tested:

“CCCC*C*”:
(SEQ ID NO: 55)
CCCCC*A*GTCAGTATCTAGTGATTATCTAGACATACATCTAGTACATGT
CTAGTCAGTATCTAGTGATTA*T*CCCCC
(SEQ ID NO: 92)
/CCCCC*A*TAATCACTAGATACTGACTAGACATGTACTAGATGTATGTC
TAGATAATCACTAGATACTGAC*T*CCCCC (Note: asterisks
denote locations of phosphorothioate linkages)
“UUUU*U*” (+UDG):
(SEQ ID NO: 220)
[abasic][abasic][abasic][abasic][abasic]*A*GTCAGTA
TCTAGTGATTATCTAGACATACATCTAGTACATGTCTAGTCAGTATCTAG
TGATTA*T*[abasic][abasic][abasic][abasic][abasic]
(SEQ ID NO: 221)
/[abasic][abasic][abasic][abasic][abasic]*A*TAATCA
CTAGATACTGACTAGACATGTACTAGATGTATGTCTAGATAATCACTAGA
TACTGAC*T*[abasic][abasic][abasic][abasic][abasic]
“CCCCU” (+UDG):
(SEQ ID NO: 180)
CCCC[abasic]AGTCAGTATCTAGTGATTATCTAGACATACATCTAGTA
CATGTCTAGTCAGTATCTAGTGATTAT[abasic]CCCC
(SEQ ID NO: 181)
/CCCC[abasic]ATAATCACTAGATACTGACTAGACATGTACTAGATGT
ATGTCTAGATAATCACTAGATACTGACT[abasic]CCCC
“CCCC*U*” (+UDG):
(SEQ ID NO: 224)
CCCC[abasic]*A*GTCAGTATCTAGTGATTATCTAGACATACATCTAG
TACATGTCTAGTCAGTATCTAGTGATTA*T*[abasic]CCCC
(SEQ ID NO: 225)
/CCCC[abasic]*A*TAATCACTAGATACTGACTAGACATGTACTAGAT
GTATGTCTAGATAATCACTAGATACTGAC*T*[abasic]CCCC

From this experiment, we concluded that combining phosphorothioate linkages at the single-stranded/double-stranded boundary regions with abasic sites in the boundary does not significantly improve potency to stimulate the SIDSP over individual factors.

1. A polynucleotide construct, comprising:
(a) a first polynucleotide at least 35 nucleotides in length, wherein the first polynucleotide has a 5′ end and a 3′ end;
(b) a second polynucleotide at least 35 nucleotides in length, wherein the first polynucleotide has a 5′ end and a 3′ end;
wherein the first polynucleotide and the second polynucleotide are base-paired to form a double-stranded region of the polynucleotide construct, wherein the double-stranded region is at least 25 contiguous base pairs in length.

2. The polynucleotide construct of claim 1, further comprising:
(c) a third polynucleotide at least 35 nucleotides in length, wherein the third polynucleotide has a 5′ end and a 3′ end; wherein
(i) the first polynucleotide and the third polynucleotide are base-paired to form a second double-stranded region of the polynucleotide construct, wherein the second double-stranded region is at least 25 contiguous base pairs in length; and
(ii) the second polynucleotide and the third polynucleotide are base-paired to form a third double-stranded region of the polynucleotide construct, wherein the second double-stranded region is at least 25 contiguous base pairs in length.

3. The polynucleotide construct of claim 1, further comprising:
(c) a third polynucleotide at least 35 nucleotides in length, wherein the third polynucleotide has a 5′ end and a 3′ end; and
(d) a fourth polynucleotide at least 35 nucleotides in length, wherein the fourth polynucleotide has a 5′ end and a 3′ end, wherein
(i) the first polynucleotide and the third polynucleotide are base-paired to form a second double-stranded region of the polynucleotide construct, wherein the second double-stranded region is at least 25 contiguous base pairs in length;
(ii) the second polynucleotide and the fourth polynucleotide are base-paired to form a third double-stranded region of the polynucleotide construct, wherein the third double-stranded region is at least 25 contiguous base pairs in length; and
(iii) the third polynucleotide and the fourth polynucleotide are base-paired to form a fourth double-stranded region of the polynucleotide construct, wherein the fourth double-stranded region is at least 25 contiguous base pairs in length.

4. The polynucleotide construct of claim 1, wherein one or more of the first polynucleotide and the second polynucleotide, comprise a single stranded region at the 5′ end and/or the 3′ end.

5. The polynucleotide construct of claim 1, wherein each double-stranded region is perfectly double-stranded, and/or wherein each double stranded region is between 25 contiguous base pairs in length and 200 contiguous base pairs in length.

6.-7. (canceled)

8. The polynucleotide construct of claim 1, wherein the double stranded region is between 45 contiguous base pairs in length and 110 contiguous base pairs in length.

9.-10. (canceled)

11. The polynucleotide construct of claim 1, wherein each single stranded region is between 3 nucleotides and 7 nucleotides in length.

12. (canceled)

13. The polynucleotide construct of claim 1, wherein one or more of the following are true:
(i) each single stranded region comprises at least one pyrimidine,
(ii) each single stranded region comprises all pyrimidines,
(iii) each single stranded region comprises at least one cytosine,
(iv) each single stranded region comprises all deoxycytosines;
(v) 1, 2, 3, 4, or more of each single stranded region comprise one or more deoxyuracil residue;
(vi) each single stranded region comprise one or more deoxyuracil residue;
(vii) 1, 2, 3, 4, or more of each single stranded region comprise one or more abasic site;
(viii) each single stranded region comprise one or more abasic site.

14.-18. (canceled)

19. The polynucleotide construct of claim 13, wherein
(A) one or more deoxyuracil residues are present at the residue in the single stranded region that abuts the double stranded region,
(B) a deoxyuracil residue is present in the middle of 1, 2, 3, 4, or more of the single stranded region;
(C) one or more abasic sites are present at the residue in the single stranded region that abuts the double stranded region; and/or
(D) an abasic site is present in the middle of 1, 2, 3, 4, or more of the single stranded regions.

20.-24. (canceled)

25. The polynucleotide construct of claim 1,
(i) comprising one or more phosphorothioate linkages between nucleotides at 1, 2, 3, 4, 5, 6, 7, or 8 boundaries of the single stranded region and the double stranded region;
(ii) comprising one or more phosphorothioate linkages between nucleotides at all boundaries of the single stranded region and the double stranded region; and/or
(iii) comprising one or more phosphorothioate linkages between nucleotides within one or more of the single stranded regions.

26.-27. (canceled)

28. The polynucleotide construct of claim 1, wherein the construct does not include phosphorothioate-linkages between nucleotides in the double-stranded regions other than at the one or more boundaries of the single stranded region and the double stranded region.

29. The polynucleotide construct of claim 1, wherein
(a) the double stranded regions have a GC nucleotide content of at least 10%, 20%, 30%, 40%, 50%, or more; or
(b) the double stranded regions have a GC nucleotide content of 20% or less, 15% or less, 10% or less, 9% or less, 8% or less, 7% or less, 6% or less, 5% or less, 4% or less, 3% or less, 2% or less, 1% or less, or 0%.

30. (canceled)

31. The polynucleotide construct of claim 1, wherein:
(i) the first polynucleotide and the second polynucleotide are of equal length and are base-paired to form a perfectly double stranded region of between 55 contiguous base pairs in length and 75 contiguous base pairs;
(ii) GC content in the double stranded region is 20% or less;
(iii) the first polynucleotide comprises a single stranded region at the 5′ end and the 3′ end, wherein each single stranded region is between 3 nucleotides and 7 nucleotides in length;
(iv) the second polynucleotide comprises a single stranded region at the 5′ end and the 3′ end, wherein each single stranded region is between 3 nucleotides and 7 nucleotides in length;
(v) there are no phosphorothioate-linkages between residues that are each in the double stranded regions; and
(v) wherein one or more of the following is true:
(A) each single stranded region comprises all pyrimidines, and there is a phosphorothioate linkages between nucleotides at all boundaries of the single stranded region and the double stranded region;
(B) each single stranded region comprises one or more abasic site, wherein (i) abasic sites are present at the residue in each single stranded region that abuts the double stranded region, and/or (ii) an abasic site is present in the middle of each of the single stranded regions.

32. The polynucleotide construct of claim 1, wherein the first the polynucleotide and the second polynucleotide comprise nucleic acid sequences at least 80% identical to the nucleic acid sequence selected from the following combinations, wherein (a) underlined residues are unpaired overhangs, (b) asterisks between bases denote individual phosphorothioate (PT) linkages, and (c) abasic residues are noted in brackets:
SEQ ID NO:1-2;
SEQ ID NO:3-4;
SEQ ID NO:5-6;
SEQ ID NO:7-8;
SEQ ID NO:9-10
SEQ ID NO:11-12;
SEQ ID NO:13-14;
SEQ ID NO:15-16;
SEQ ID NO:17-18;
SEQ ID NO:19-20;
SEQ ID NO:21-22;
SEQ ID NO:23-24
SEQ ID NO:25-26;
SEQ ID NO:27-28;
SEQ ID NO:29-30;
SEQ ID NO:35-36;
SEQ ID NO: 35 and SEQ ID NO:32;
SEQ ID NO:41-42;
SEQ ID NO: 41 and SEQ ID NO:44;
SEQ ID NO: 46 and SEQ ID NO:42;
SEQ ID NO: 46 and SEQ ID NO:44;
SEQ ID NO:51 and SEQ ID NO:22;
SEQ ID NO:53- and SEQ ID NO:22;
SEQ ID NO:55 and SEQ ID NO:22;
SEQ ID NO:59 and SEQ ID NO:22;
SEQ ID NO:61 and SEQ ID NO:22;
SEQ ID NO: 59 and SEQ ID NO:36;
SEQ ID NO: 61 and SEQ ID NO:36;
SEQ ID NO: 53 and SEQ ID NO:36;
SEQ ID NO: 55 and SEQ ID NO:36;
SEQ ID NO:81 and SEQ ID NO:22;
SEQ ID NO:83 and SEQ ID NO:22;
SEQ ID NO:85 and SEQ ID NO:22;
SEQ ID NO:87 and SEQ ID NO:22;
SEQ ID NO:89 and SEQ ID NO:22;
SEQ ID NO: 51 and SEQ ID NO:92;
SEQ ID NO: 81 and SEQ ID NO:92;
SEQ ID NO: 83 and SEQ ID NO:92;
SEQ ID NO: 85 and SEQ ID NO:92;
SEQ ID NO: 87 and SEQ ID NO:92;
SEQ ID NO: 89 and SEQ ID NO:92;
SEQ ID NO: 55 and SEQ ID NO:92;
SEQ ID NO:105-106;
SEQ ID NO:107-108;
SEQ ID NO:125-126;
SEQ ID NO:127-128
SEQ ID NO:129-130;
SEQ ID NO:131-132;
SEQ ID NO:135-136;
SEQ ID NO:137-138;
SEQ ID NO:139-140;
SEQ ID NO:154-155;
SEQ ID NO:156-157;
SEQ ID NO:160-161;
SEQ ID NO:162-163;
SEQ ID NO:164-165;
SEQ ID NO:168-169;
SEQ ID NO:174-175;
SEQ ID NO:176-177;
SEQ ID NO:178-179;
SEQ ID NO:180-181;
SEQ ID NO:184-185;
SEQ ID NO:190-191;
SEQ ID NO:196-197;
SEQ ID NO:198-199;
SEQ ID NO:206-207;
SEQ ID NO:214-215;
SEQ ID NO:216-217;
SEQ ID NO:220-221;
SEQ ID NO:224-225;
SEQ ID NO:145-147 (Triplex); and
SEQ ID NO: 145, 146, 149, and 151 (Quadruplex).

33.-34. (canceled)

35. A polynucleotide comprising a nucleic acid sequence at least 80% identical to the nucleic acid sequence selected from the group consisting of SEQ ID NOS:1-30, 33-36, 41-42, 44-45, 51, 53, 55, 59, 61, 81, 83, 85, 87, 89, 92, 105-108, 125-132, 135-140, 145-147, 149, 151, 154-165, 168-169, 174-181, 184-185, 190-191, 196-199, 206-207, 214-217, 220-221, and 224-225, wherein (a) underlined residues are unpaired overhangs, (b) asterisks between bases denote individual phosphorothioate (PT) linkages, and (c) abasic residues are noted in brackets.

36. An expression vector comprising one or more of the polynucleotides of claim 35 operatively linked to a control sequence.

37. A host cell comprising the expression vector of claim 36.

38. A pharmaceutical composition comprising the polynucleotide construct of claim 1.

39. A method for treating autoimmune disorders, infections (such as viral infections), tumors, or for stimulating an immune response, comprising administering to a subject in need thereof an amount effective to treat the autoimmune disorders, infections (such as viral infections), or tumors, or for stimulating an immune response of the polynucleotide construct of claim 1.

40. (canceled)

41. A kit comprising
(a) a first polynucleotide at least 35 nucleotides in length, wherein the first polynucleotide has a 5′ end and a 3′ end;
(b) a second polynucleotide at least 35 nucleotides in length, wherein the first polynucleotide has a 5′ end and a 3′ end;
wherein the first polynucleotide and the second polynucleotide are capable of base-pairing to form a double-stranded region of at least 25 contiguous base pairs in length; and
wherein optionally one or both of the first polynucleotide and the second polynucleotide comprise a single stranded region at the 5′ end and/or the 3′ end; and
(c) optionally a third polynucleotide at least 35 nucleotides in length, wherein the third polynucleotide has a 5′ end and a 3′ end; wherein
(i) the first polynucleotide and the third polynucleotide are capable of base pairing to form a second double-stranded region of at least 25 contiguous base pairs in length; and
(ii) the second polynucleotide and the third polynucleotide are capable of base pairing to form a third double-stranded region at least 25 contiguous base pairs in length;
wherein optionally the third polynucleotide may comprise a single stranded region at the 5′ end and/or the 3′; or
(d) optionally, (i) a third polynucleotide at least 35 nucleotides in length, wherein the third polynucleotide has a 5′ end and a 3′ end; and (ii) a fourth polynucleotide at least 35 nucleotides in length, wherein the fourth polynucleotide has a 5′ end and a 3′ end, wherein
(A) the first polynucleotide and the third polynucleotide are capable of base pairing to form a second double-stranded region at least 25 contiguous base pairs in length;
(B) the second polynucleotide and the fourth polynucleotide are capable of base pairing to form a third double-stranded region at least 25 contiguous base pairs in length; and
(C) the third polynucleotide and the fourth polynucleotide are capable of base pairing to form a fourth double-stranded region of at least 25 contiguous base pairs in length
wherein the third polynucleotide and/or the fourth polynucleotide may optionally comprise a single stranded region at the 5′ end and/or the 3′ end.

42.-71. (canceled)


SEQUENCE LISTING (283 sequences)

SEQ ID NO: 1 - DNA, 45 bp, Artificial Sequence
  Synthetic oligonucleotide
aaaaaaggtcccaccagggtccacctggggtcacctgggaaaaaa

SEQ ID NO: 2 - DNA, 45 bp, Artificial Sequence
  Synthetic oligonucleotide
aaaaatcccaggtgaccccaggtggaccctggtgggacctaaaaa

SEQ ID NO: 3 - DNA, 45 bp, Artificial Sequence
  Synthetic oligonucleotide
cccccaggtcccaccagggtccacctggggtcacctgggaccccc

SEQ ID NO: 4 - DNA, 45 bp, Artificial Sequence
  Synthetic oligonucleotide
ccccctcccaggtgaccccaggtggaccctggtgggacctccccc

SEQ ID NO: 5 - DNA, 41 bp, Artificial Sequence
  Synthetic oligonucleotide
aaaaggtcccaccagggtccacctggggtcacctgggaaaa

SEQ ID NO: 6 - DNA, 41 bp, Artificial Sequence
  Synthetic oligonucleotide
aaatcccaggtgaccccaggtggaccctggtgggacctaaa

SEQ ID NO: 7 - DNA, 41 bp, Artificial Sequence
  Synthetic oligonucleotide
cccaggtcccaccagggtccacctggggtcacctgggaccc

SEQ ID NO: 8 - DNA, 41 bp, Artificial Sequence
  Synthetic oligonucleotide
ccctcccaggtgaccccaggtggaccctggtgggacctccc

SEQ ID NO: 9 - DNA, 55 bp, Artificial Sequence
  Synthetic oligonucleotide
ccccctagtgattatctagacatacatctagtacatgtctagtcagtataccccc

SEQ ID NO: 10 - DNA, 55 bp, Artificial Sequence
  Synthetic oligonucleotide
ccccctatactgactagacatgtactagatgtatgtctagataatcactaccccc

SEQ ID NO: 11 - DNA, 60 bp, Artificial Sequence
  Synthetic oligonucleotide
cccccatctagtgattatctagacatacatctagtacatgtctagtcagtatctaccccc

SEQ ID NO: 12 - DNA, 60 bp, Artificial Sequence
  Synthetic oligonucleotide
ccccctagatactgactagacatgtactagatgtatgtctagataatcactagatccccc

SEQ ID NO: 13 - DNA, 55 bp, Artificial Sequence
  Synthetic oligonucleotide
agtatctagtgattatctagacatacatctagtacatgtctagtcagtatctagt

SEQ ID NO: 14 - DNA, 55 bp, Artificial Sequence
  Synthetic oligonucleotide
actagatactgactagacatgtactagatgtatgtctagataatcactagatact

SEQ ID NO: 15 - DNA, 65 bp, Artificial Sequence
  Synthetic oligonucleotide
cccccagtatctagtgattatctagacatacatctagtacatgtctagtcagtatctagtccccc

SEQ ID NO: 16 - DNA, 65 bp, Artificial Sequence
  Synthetic oligonucleotide
cccccactagatactgactagacatgtactagatgtatgtctagataatcactagatactccccc

SEQ ID NO: 17 - DNA, 60 bp, Artificial Sequence
  Synthetic oligonucleotide
tcagtatctagtgattatctagacatacatctagtacatgtctagtcagtatctagtgat

SEQ ID NO: 18 - DNA, 60 bp, Artificial Sequence
  Synthetic oligonucleotide
atcactagatactgactagacatgtactagatgtatgtctagataatcactagatactga

SEQ ID NO: 19 - DNA, 70 bp, Artificial Sequence
  Synthetic oligonucleotide
ccccctcagtatctagtgattatctagacatacatctagtacatgtctagtcagtatctagtgatccccc

SEQ ID NO: 20 - DNA, 70 bp, Artificial Sequence
  Synthetic oligonucleotide
cccccatcactagatactgactagacatgtactagatgtatgtctagataatcactagatactgaccccc

SEQ ID NO: 21 - DNA, 75 bp, Artificial Sequence
  Synthetic oligonucleotide
cccccagtcagtatctagtgattatctagacatacatctagtacatgtctagtcagtatctagtgattatccccc

SEQ ID NO: 22 - DNA, 75 bp, Artificial Sequence
  Synthetic oligonucleotide
cccccataatcactagatactgactagacatgtactagatgtatgtctagataatcactagatactgactccccc

SEQ ID NO: 23 - DNA, 65 bp, Artificial Sequence
  Synthetic oligonucleotide
agtcagtatctagtgattatctagacatacatctagtacatgtctagtcagtatctagtgattat

SEQ ID NO: 24 - DNA, 65 bp, Artificial Sequence
  Synthetic oligonucleotide
ataatcactagatactgactagacatgtactagatgtatgtctagataatcactagatactgact

SEQ ID NO: 25 - DNA, 90 bp, Artificial Sequence
  Synthetic oligonucleotide
cccccaatgtctagtcagtatctagtgattatctagacatacatctagtacatgtctagtcagtatctagtgattatctagacatccccc

SEQ ID NO: 26 - DNA, 90 bp, Artificial Sequence
  Synthetic oligonucleotide
cccccatgtctagataatcactagatactgactagacatgtactagatgtatgtctagataatcactagatactgactagacattccccc

SEQ ID NO: 27 - DNA, 80 bp, Artificial Sequence
  Synthetic oligonucleotide
aatgtctagtcagtatctagtgattatctagacatacatctagtacatgtctagtcagtatctagtgattatctagacat

SEQ ID NO: 28 - DNA, 80 bp, Artificial Sequence
  Synthetic oligonucleotide
atgtctagataatcactagatactgactagacatgtactagatgtatgtctagataatcactagatactgactagacatt

SEQ ID NO: 29 - DNA, 100 bp, Artificial Sequence
  Synthetic oligonucleotide
acatctagtacatgtctagtcagtatctagtgattatctagacatacatctagtacatgtctagtcagtatctagtgattatctagacatggactcatcc

SEQ ID NO: 30 - DNA, 100 bp, Artificial Sequence
  Synthetic oligonucleotide
ggatgagtccatgtctagataatcactagatactgactagacatgtactagatgtatgtctagataatcactagatactgactagacatgtactagatgt

SEQ ID NO: 31-34 - skipped (4 entries)

SEQ ID NO: 35 - DNA, 75 bp, Artificial Sequence; identical to SEQ ID NO: 21
  Synthetic oligonucleotide
  misc_feature (1)(11): phosphorothioate linkages
  misc_feature (65)(75): phosphorothioate linkages

SEQ ID NO: 36 - DNA, 75 bp, Artificial Sequence; identical to SEQ ID NO: 22
  Synthetic oligonucleotide
  misc_feature (1)(11): phosphorothioate linkages
  misc_feature (65)(75): phosphorothioate linkages

SEQ ID NO: 37-40 - skipped (4 entries)

SEQ ID NO: 41 - DNA, 70 bp, Artificial Sequence
  Synthetic oligonucleotide
cccccagtcagtatctagtgattatctagacatacatctagtacatgtctagtcagtatctagtgattat

SEQ ID NO: 42 - DNA, 70 bp, Artificial Sequence
  Synthetic oligonucleotide
ataatcactagatactgactagacatgtactagatgtatgtctagataatcactagatactgactccccc

SEQ ID NO: 43 - skipped

SEQ ID NO: 44 - DNA, 70 bp, Artificial Sequence
  Synthetic oligonucleotide
cccccataatcactagatactgactagacatgtactagatgtatgtctagataatcactagatactgact

SEQ ID NO: 45 - DNA, 70 bp, Artificial Sequence
  Synthetic oligonucleotide
agtcagtatctagtgattatctagacatacatctagtacatgtctagtcagtatctagtgattatccccc

SEQ ID NO: 46 - skipped

SEQ ID NO: 47 - DNA, 70 bp, Artificial Sequence; identical to SEQ ID NO: 45
  Synthetic oligonucleotide

SEQ ID NO: 48-50 - skipped (3 entries)

SEQ ID NO: 51 - DNA, 75 bp, Artificial Sequence; identical to SEQ ID NO: 21
  Synthetic oligonucleotide
  misc_feature (35)(40): phosphorothioate linkages

SEQ ID NO: 52 - skipped

SEQ ID NO: 53 - DNA, 75 bp, Artificial Sequence; identical to SEQ ID NO: 21
  Synthetic oligonucleotide
  misc_feature (1)(2): phosphorothioate linkage
  misc_feature (74)(74): phosphorothioate linkage

SEQ ID NO: 54 - skipped

SEQ ID NO: 55 - DNA, 75 bp, Artificial Sequence; identical to SEQ ID NO: 21
  Synthetic oligonucleotide
  misc_feature (4)(6): phosphorothioate linkages
  misc_feature (70)(72): phosphorothioate linkages

SEQ ID NO: 56-58 - skipped (3 entries)

SEQ ID NO: 59 - DNA, 75 bp, Artificial Sequence; identical to SEQ ID NO: 21
  Synthetic oligonucleotide
  misc_feature (1)(6): phosphorothioate linkages
  misc_feature (70)(75): phosphorothioate linkages

SEQ ID NO: 60 - skipped

SEQ ID NO: 61 - DNA, 75 bp, Artificial Sequence; identical to SEQ ID NO: 21
  Synthetic oligonucleotide
  misc_feature (1)(4): phosphorothioate linkages
  misc_feature (71)(75): phosphorothioate linkages

SEQ ID NO: 62-80 - skipped (19 entries)

SEQ ID NO: 81 - DNA, 75 bp, Artificial Sequence; identical to SEQ ID NO: 21
  Synthetic oligonucleotide
  misc_feature (36)(39): phosphorothioate linkages

SEQ ID NO: 82 - skipped

SEQ ID NO: 83 - DNA, 75 bp, Artificial Sequence; identical to SEQ ID NO: 21
  Synthetic oligonucleotide
  misc_feature (37)(38): phosphorothioate linkage

SEQ ID NO: 84 - skipped

SEQ ID NO: 85 - DNA, 75 bp, Artificial Sequence; identical to SEQ ID NO: 21
  Synthetic oligonucleotide
  misc_feature (5)(7): phosphorothioate linkages
  misc_feature (36)(41): phosphorothioate linkages
  misc_feature (69)(71): phosphorothioate linkages

SEQ ID NO: 86 - skipped

SEQ ID NO: 87 - DNA, 75 bp, Artificial Sequence; identical to SEQ ID NO: 21
  Synthetic oligonucleotide
  misc_feature (5)(7): phosphorothioate linkages
  misc_feature (37)(40): phosphorothioate linkages
  misc_feature (69)(71): phosphorothioate linkages

SEQ ID NO: 88 - skipped

SEQ ID NO: 89 - DNA, 75 bp, Artificial Sequence; identical to SEQ ID NO: 21
  Synthetic oligonucleotide
  misc_feature (5)(7): phosphorothioate linkages
  misc_feature (37)(38): phosphorothioate linkage
  misc_feature (69)(71): phosphorothioate linkages

SEQ ID NO: 90-91 - skipped (2 entries)

SEQ ID NO: 92 - DNA, 75 bp, Artificial Sequence; identical to SEQ ID NO: 22
  Synthetic oligonucleotide
  misc_feature (5)(7): phosphorothioate linkages
  misc_feature (69)(71): phosphorothioate linkages

SEQ ID NO: 93-101 - skipped (9 entries)

SEQ ID NO: 102 - DNA, 75 bp, Artificial Sequence; identical to SEQ ID NO: 22
  Synthetic oligonucleotide

SEQ ID NO: 103-104 - skipped (2 entries)

SEQ ID NO: 105 - DNA, 75 bp, Artificial Sequence; identical to SEQ ID NO: 21
  Synthetic oligonucleotide
  misc_feature (5)(6): phosphorothioate linkage
  misc_feature (70)(71): phosphorothioate linkage

SEQ ID NO: 106 - DNA, 75 bp, Artificial Sequence; identical to SEQ ID NO: 22
  Synthetic oligonucleotide
  misc_feature (5)(6): phosphorothioate linkage
  misc_feature (70)(71): phosphorothioate linkage

SEQ ID NO: 107 - DNA, 75 bp, Artificial Sequence; identical to SEQ ID NO: 21
  Synthetic oligonucleotide
  misc_feature (6)(7): phosphorothioate linkage
  misc_feature (69)(70): phosphorothioate linkage

SEQ ID NO: 108 - DNA, 75 bp, Artificial Sequence; identical to SEQ ID NO: 22
  Synthetic oligonucleotide
  misc_feature (6)(7): phosphorothioate linkage
  misc_feature (69)(70): phosphorothioate linkage

SEQ ID NO: 109-124 - skipped (16 entries)

SEQ ID NO: 125 - DNA, 75 bp, Artificial Sequence; identical to SEQ ID NO: 21
  Synthetic oligonucleotide
  misc_feature (1)(2): phosphorothioate linkage
  misc_feature (5)(7): phosphorothioate linkages
  misc_feature (69)(71): phosphorothioate linkages
  misc_feature (74)(75): phosphorothioate linkage

SEQ ID NO: 126 - DNA, 75 bp, Artificial Sequence; identical to SEQ ID NO: 22
  Synthetic oligonucleotide
  misc_feature (1)(2): phosphorothioate linkage
  misc_feature (5)(7): phosphorothioate linkages
  misc_feature (69)(71): phosphorothioate linkages
  misc_feature (74)(75): phosphorothioate linkage

SEQ ID NO: 127 - DNA, 65 bp, Artificial Sequence; identical to SEQ ID NO: 23
  Synthetic oligonucleotide

SEQ ID NO: 128 - DNA, 65 bp, Artificial Sequence; identical to SEQ ID NO: 24
  Synthetic oligonucleotide

SEQ ID NO: 129 - DNA, 67 bp, Artificial Sequence
  Synthetic oligonucleotide
cagtcagtatctagtgattatctagacatacatctagtacatgtctagtcagtatctagtgattatc

SEQ ID NO: 130 - DNA, 67 bp, Artificial Sequence
  Synthetic oligonucleotide
cataatcactagatactgactagacatgtactagatgtatgtctagataatcactagatactgactc

SEQ ID NO: 131 - DNA, 71 bp, Artificial Sequence
  Synthetic oligonucleotide
cccagtcagtatctagtgattatctagacatacatctagtacatgtctagtcagtatctagtgattatccc

SEQ ID NO: 132 - DNA, 71 bp, Artificial Sequence
  Synthetic oligonucleotide
cccataatcactagatactgactagacatgtactagatgtatgtctagataatcactagatactgactccc

SEQ ID NO: 133-134 - skipped (2 entries)

SEQ ID NO: 135 - DNA, 81 bp, Artificial Sequence
  Synthetic oligonucleotide
ccccccccagtcagtatctagtgattatctagacatacatctagtacatgtctagtcagtatctagtgattatcccccccc

SEQ ID NO: 136 - DNA, 81 bp, Artificial Sequence
  Synthetic oligonucleotide
ccccccccataatcactagatactgactagacatgtactagatgtatgtctagataatcactagatactgactcccccccc

SEQ ID NO: 137 - DNA, 89 bp, Artificial Sequence
  Synthetic oligonucleotide
ccccccccccccagtcagtatctagtgattatctagacatacatctagtacatgtctagtcagtatctagtgattatcccccccccccc

SEQ ID NO: 138 - DNA, 89 bp, Artificial Sequence
  Synthetic oligonucleotide
ccccccccccccataatcactagatactgactagacatgtactagatgtatgtctagataatcactagatactgactcccccccccccc

SEQ ID NO: 139 - DNA, 75 bp, Artificial Sequence
  Synthetic oligonucleotide
aaaaaagtcagtatctagtgattatctagacatacatctagtacatgtctagtcagtatctagtgattataaaaa

SEQ ID NO: 140 - DNA, 75 bp, Artificial Sequence
  Synthetic oligonucleotide
aaaaaataatcactagatactgactagacatgtactagatgtatgtctagataatcactagatactgactaaaaa

SEQ ID NO: 141-144 - skipped (4 entries)

SEQ ID NO: 145 - DNA, 75 bp, Artificial Sequence
  Synthetic oligonucleotide
  misc_feature (5)(7): phosphorothioate linkages
  misc_feature (69)(71): phosphorothioate linkages
cccccagtcagtatctagtgattatctagacatacatctagtacatgtctagtcagtatgtagtgattatccccc

SEQ ID NO: 146 - DNA, 75 bp, Artificial Sequence
  Synthetic oligonucleotide
  misc_feature (5)(7): phosphorothioate linkages
  misc_feature (69)(71): phosphorothioate linkages
cccccataatcactacatactgactagacatgtactagactctgtttcaagagaataacttgagattcaaccccc

SEQ ID NO: 147 - DNA, 75 bp, Artificial Sequence
  Synthetic oligonucleotide
  misc_feature (5)(7): phosphorothioate linkages
  misc_feature (69)(71): phosphorothioate linkages
cccccttgaatctcaagttattctcttgaaacagagtatgtatgtctagataatcactagatactgacttccccc

SEQ ID NO: 148 - skipped

SEQ ID NO: 149 - DNA, 75 bp, Artificial Sequence
  Synthetic oligonucleotide
  misc_feature (5)(7): phosphorothioate linkages
  misc_feature (69)(71): phosphorothioate linkages
cccccatgtgagaatgtactgttgttcaagtcaaacttatgtatgtctagataatcactagatactgactccccc

SEQ ID NO: 150 - skipped

SEQ ID NO: 151 - DNA, 75 bp, Artificial Sequence
  Synthetic oligonucleotide
  misc_feature (5)(7): phosphorothioate linkages
  misc_feature (69)(71): phosphorothioate linkages
cccccttgaatctcaagttattctcttgaaacagagtaagtttgacttgaacaacagtacattctcacatccccc

SEQ ID NO: 152-153 - skipped (2 entries)

SEQ ID NO: 154 - DNA, 75 bp, Artificial Sequence
  Synthetic oligonucleotide
cccccagtcagcctccagtgagcatctagacatgcatctagtgcctgtccagccagcctctggtgattatccccc

SEQ ID NO: 155 - DNA, 75 bp, Artificial Sequence
  Synthetic oligonucleotide
cccccataatcaccagaggctggctggacaggcactagatgcatgtctagatgctcactggaggctgactccccc

SEQ ID NO: 156 - DNA, 75 bp, Artificial Sequence
  Synthetic oligonucleotide
ccccctgaatgaacaatcatctatagacagatacagtattatcaatgtaatgtacaatctttatgagcttccccc

SEQ ID NO: 157 - DNA, 75 bp, Artificial Sequence
  Synthetic oligonucleotide
cccccaagctcataaagattgtacattacattgataatactgtatctgtctatagatgattgttcattcaccccc

SEQ ID NO: 158-159 - skipped (2 entries)

SEQ ID NO: 160 - DNA, 75 bp, Artificial Sequence
  Synthetic oligonucleotide
cccccatatatatatatatatatatatatatatatatatatatatatatatatatatatatatatatataccccc

SEQ ID NO: 161 - DNA, 75 bp, Artificial Sequence
  Synthetic oligonucleotide
ccccctatatatatatatatatatatatatatatatatatatatatatatatatatatatatatatatatccccc

SEQ ID NO: 162 - DNA, 75 bp, Artificial Sequence
  Synthetic oligonucleotide
cccccatatatatatatatatatatacatatatatatatatatatatagatatatatatatatatatataccccc

SEQ ID NO: 163 - DNA, 75 bp, Artificial Sequence
  Synthetic oligonucleotide
ccccctatatatatatatatatatatctatatatatatatatatatatgtatatatatatatatatatatccccc

SEQ ID NO: 164 - DNA, 75 bp, Artificial Sequence
  Synthetic oligonucleotide
cccccatatactatatgatatagtatatcatatactatatgatatagtatatcatatactatatgatataccccc

SEQ ID NO: 165 - DNA, 75 bp, Artificial Sequence
  Synthetic oligonucleotide
ccccctatatcatatagtatatgatatactatatcatatagtatatgatatactatatcatatagtatatccccc

SEQ ID NO: 166-167 - skipped (2 entries)

SEQ ID NO: 168 - DNA, 75 bp, Artificial Sequence
  Synthetic oligonucleotide
  misc_feature (1)(5): Abasic sites generated from UDG treatment of deoxyuracil-containing DNA
  misc_feature (71)(75): Abasic sites generated from UDG treatment of deoxyuracil-containing DNA
nnnnnagtcagtatctagtgattatctagacatacatctagtacatgtctagtcagtatctagtgattatnnnnn

SEQ ID NO: 169 - DNA, 75 bp, Artificial Sequence
  Synthetic oligonucleotide
  misc_feature (1)(5): Abasic sites generated from UDG treatment of deoxyuracil-containing DNA
  misc_feature (71)(75): Abasic sites generated from UDG treatment of deoxyuracil-containing DNA
nnnnnataatcactagatactgactagacatgtactagatgtatgtctagataatcactagatactgactnnnnn

SEQ ID NO: 170-173 - skipped (4 entries)

SEQ ID NO: 174 - DNA, 75 bp, Artificial Sequence
  Synthetic oligonucleotide
uccccagtcagtatctagtgattatctagacatacatctagtacatgtctagtcagtatctagtgattatccccu

SEQ ID NO: 175 - DNA, 75 bp, Artificial Sequence
  Synthetic oligonucleotide
uccccataatcactagatactgactagacatgtactagatgtatgtctagataatcactagatactgactccccu

SEQ ID NO: 176 - DNA, 75 bp, Artificial Sequence
  Synthetic oligonucleotide
  misc_feature (1)(1): Abasic site generated from UDG treatment of deoxyuracil-containing DNA
  misc_feature (75)(75): Abasic site generated from UDG treatment of deoxyuracil-containing DNA
nccccagtcagtatctagtgattatctagacatacatctagtacatgtctagtcagtatctagtgattatccccn

SEQ ID NO: 177 - DNA, 75 bp, Artificial Sequence
  Synthetic oligonucleotide
  misc_feature (1)(1): Abasic site generated from UDG treatment of deoxyuracil-containing DNA
  misc_feature (75)(75): Abasic site generated from UDG treatment of deoxyuracil-containing DNA
nccccataatcactagatactgactagacatgtactagatgtatgtctagataatcactagatactgactccccn

SEQ ID NO: 178 - DNA, 75 bp, Artificial Sequence
  Synthetic oligonucleotide
ccccuagtcagtatctagtgattatctagacatacatctagtacatgtctagtcagtatctagtgattatucccc

SEQ ID NO: 179 - DNA, 75 bp, Artificial Sequence
  Synthetic oligonucleotide
ccccuataatcactagatactgactagacatgtactagatgtatgtctagataatcactagatactgactucccc

SEQ ID NO: 180 - DNA, 75 bp, Artificial Sequence
  Synthetic oligonucleotide
  misc_feature (5)(5): Abasic site generated from UDG treatment of deoxyuracil-containing DNA
  misc_feature (71)(71): Abasic site generated from UDG treatment of deoxyuracil-containing DNA
ccccnagtcagtatctagtgattatctagacatacatctagtacatgtctagtcagtatctagtgattatncccc

SEQ ID NO: 181 - DNA, 75 bp, Artificial Sequence
  Synthetic oligonucleotide
  misc_feature (5)(5): Abasic site generated from UDG treatment of deoxyuracil-containing DNA
  misc_feature (71)(71): Abasic site generated from UDG treatment of deoxyuracil-containing DNA
ccccnataatcactagatactgactagacatgtactagatgtatgtctagataatcactagatactgactncccc

SEQ ID NO: 182-183 - skipped (2 entries)

SEQ ID NO: 184 - DNA, 73 bp, Artificial Sequence
  Synthetic oligonucleotide
  misc_feature (4)(5): Int 1 2 Dideoxyribose (dSpacer)
  misc_feature (69)(70): Int 1 2 Dideoxyribose (dSpacer)
ccccagtcagtatctagtgattatctagacatacatctagtacatgtctagtcagtatctagtgattatcccc

SEQ ID NO: 185 - DNA, 73 bp, Artificial Sequence
  Synthetic oligonucleotide
  misc_feature (4)(5): Int 1 2 Dideoxyribose (dSpacer)
  misc_feature (69)(70): Int 1 2 Dideoxyribose (dSpacer)
ccccataatcactagatactgactagacatgtactagatgtatgtctagataatcactagatactgactcccc

SEQ ID NO: 186-189 - skipped (4 entries)

SEQ ID NO: 190 - DNA, 75 bp, Artificial Sequence
  Synthetic oligonucleotide
  misc_feature (5)(5): Abasic site generated from UDG treatment of deoxyuracil-containing DNA
  misc_feature (71)(71): Abasic site generated from UDG treatment of deoxyuracil-containing DNA
ttttnagtcagtatctagtgattatctagacatacatctagtacatgtctagtcagtatctagtgattatntttt

SEQ ID NO: 191 - DNA, 75 bp, Artificial Sequence
  Synthetic oligonucleotide
  misc_feature (5)(5): Abasic site generated from UDG treatment of deoxyuracil-containing DNA
  misc_feature (71)(71): Abasic site generated from UDG treatment of deoxyuracil-containing DNA
ttttnataatcactagatactgactagacatgtactagatgtatgtctagataatcactagatactgactntttt

SEQ ID NO: 192-195 - skipped (4 entries)

SEQ ID NO: 196 - DNA, 75 bp, Artificial Sequence
  Synthetic oligonucleotide
  misc_feature (5)(5): Abasic site generated from UDG treatment of deoxyuracil-containing DNA
ccccnagtcagtatctagtgattatctagacatacatctagtacatgtctagtcagtatctagtgattatccccc

SEQ ID NO: 197 - DNA, 75 bp, Artificial Sequence
  Synthetic oligonucleotide
  misc_feature (5)(5): Abasic site generated from UDG treatment of deoxyuracil-containing DNA
ccccnataatcactagatactgactagacatgtactagatgtatgtctagataatcactagatactgactccccc

SEQ ID NO: 198 - DNA, 75 bp, Artificial Sequence
  Synthetic oligonucleotide
  misc_feature (71)(71): Abasic site generated from UDG treatment of deoxyuracil-containing DNA
cccccagtcagtatctagtgattatctagacatacatctagtacatgtctagtcagtatctagtgattatncccc

SEQ ID NO: 199 - DNA, 76 bp, Artificial Sequence
  Synthetic oligonucleotide
  misc_feature (71)(71): Abasic site generated from UDG treatment of deoxyuracil-containing DNA
cccccataatcactagatactgactagacatgtactagatgtatgtctagataatcactagatactgactnccccc

SEQ ID NO: 200-205 - skipped (6 entries)

SEQ ID NO: 206 - DNA, 50 bp, Artificial Sequence
  Synthetic oligonucleotide
  misc_feature (5)(5): Abasic site generated from UDG treatment of deoxyuracil-containing DNA
  misc_feature (46)(46): Abasic site generated from UDG treatment of deoxyuracil-containing DNA
ccccnagtcagtatctagtgattatagtcagtatctagtgattatncccc

SEQ ID NO: 207 - DNA, 50 bp, Artificial Sequence
  Synthetic oligonucleotide
  misc_feature (5)(5): Abasic site generated from UDG treatment of deoxyuracil-containing DNA
  misc_feature (46)(46): Abasic site generated from UDG treatment of deoxyuracil-containing DNA
ccccnataatcactagatactgactataatcactagatactgactncccc

SEQ ID NO: 208-213 - skipped (6 entries)

SEQ ID NO: 214 - DNA, 75 bp, Artificial Sequence
  Synthetic oligonucleotide
ccuccagtcagtatctagtgattatctagacatacatctagtacatgtctagtcagtatctagtgattatccucc

SEQ ID NO: 215 - DNA, 75 bp, Artificial Sequence
  Synthetic oligonucleotide
ccuccataatcactagatactgactagacatgtactagatgtatgtctagataatcactagatactgactccucc

SEQ ID NO: 216 - DNA, 75 bp, Artificial Sequence
  Synthetic oligonucleotide
  misc_feature (3)(3): Abasic site generated from UDG treatment of deoxyuracil-containing DNA
  misc_feature (73)(73): Abasic site generated from UDG treatment of deoxyuracil-containing DNA
ccnccagtcagtatctagtgattatctagacatacatctagtacatgtctagtcagtatctagtgattatccncc

SEQ ID NO: 217 - DNA, 75 bp, Artificial Sequence
  Synthetic oligonucleotide
  misc_feature (3)(3): Abasic site generated from UDG treatment of deoxyuracil-containing DNA
  misc_feature (73)(73): Abasic site generated from UDG treatment of deoxyuracil-containing DNA
ccnccataatcactagatactgactagacatgtactagatgtatgtctagataatcactagatactgactccncc

SEQ ID NO: 218-219 - skipped (2 entries)

SEQ ID NO: 220 - DNA, 75 bp, Artificial Sequence; identical to SEQ ID NO: 168
  Synthetic oligonucleotide
  misc_feature (1)(5): Abasic sites generated from UDG treatment of deoxyuracil-containing DNA
  misc_feature (5)(7): phosphorothioate linkages
  misc_feature (69)(71): phosphorothioate linkages
  misc_feature (71)(75): Abasic sites generated from UDG treatment of deoxyuracil-containing DNA

SEQ ID NO: 221 - DNA, 75 bp, Artificial Sequence; identical to SEQ ID NO: 169
  Synthetic oligonucleotide
  misc_feature (1)(5): Abasic sites generated from UDG treatment of deoxyuracil-containing DNA
  misc_feature (5)(7): phosphorothioate linkages
  misc_feature (69)(71): phosphorothioate linkages
  misc_feature (71)(75): Abasic sites generated from UDG treatment of deoxyuracil-containing DNA

SEQ ID NO: 222-223 - skipped (2 entries)

SEQ ID NO: 224 - DNA, 75 bp, Artificial Sequence; identical to SEQ ID NO: 180
  Synthetic oligonucleotide
  misc_feature (5)(5): Abasic site generated from UDG treatment of deoxyuracil-containing DNA
  misc_feature (5)(7): phosphorothioate linkages
  misc_feature (69)(71): phosphorothioate linkages
  misc_feature (71)(71): Abasic site generated from UDG treatment of deoxyuracil-containing DNA

SEQ ID NO: 225 - DNA, 75 bp, Artificial Sequence; identical to SEQ ID NO: 181
  Synthetic oligonucleotide
  misc_feature (5)(5): Abasic site generated from UDG treatment of deoxyuracil-containing DNA
  misc_feature (5)(7): phosphorothioate linkages
  misc_feature (69)(71): phosphorothioate linkages
  misc_feature (71)(71): Abasic site generated from UDG treatment of deoxyuracil-containing DNA

SEQ ID NO: 226 - DNA, 45 bp, Artificial Sequence
  Synthetic oligonucleotide
tttttaggtcccaccagggtccacctggggtcacctgggattttt

SEQ ID NO: 227 - DNA, 45 bp, Artificial Sequence
  Synthetic oligonucleotide
ttttttcccaggtgaccccaggtggaccctggtgggacctttttt

SEQ ID NO: 228 - DNA, 45 bp, Artificial Sequence
  Synthetic oligonucleotide
gggggaggtcccaccagggtccacctggggtcacctgggaggggg

SEQ ID NO: 229 - DNA, 45 bp, Artificial Sequence
  Synthetic oligonucleotide
gggggtcccaggtgaccccaggtggaccctggtgggacctggggg

SEQ ID NO: 230 - DNA, 41 bp, Artificial Sequence
  Synthetic oligonucleotide
tttaggtcccaccagggtccacctggggtcacctgggattt

SEQ ID NO: 231 - DNA, 41 bp, Artificial Sequence
  Synthetic oligonucleotide
ttttcccaggtgaccccaggtggaccctggtgggacctttt

SEQ ID NO: 232 - DNA, 41 bp, Artificial Sequence
  Synthetic oligonucleotide
gggaggtcccaccagggtccacctggggtcacctgggaggg

SEQ ID NO: 233 - DNA, 41 bp, Artificial Sequence
  Synthetic oligonucleotide
gggtcccaggtgaccccaggtggaccctggtgggacctggg

SEQ ID NO: 234-235 - skipped (2 entries)

SEQ ID NO: 236 - DNA, 40 bp, Artificial Sequence
  Synthetic oligonucleotide
cccccacaccagggtccacctggggtcacctgggaccccc

SEQ ID NO: 237 - DNA, 40 bp, Artificial Sequence
  Synthetic oligonucleotide
ccccctcccaggtgaccccaggtggaccctggtgtccccc

SEQ ID NO: 238 - DNA, 35 bp, Artificial Sequence
  Synthetic oligonucleotide
cccccagggtccacctggggtcacctgggaccccc

SEQ ID NO: 239 - DNA, 35 bp, Artificial Sequence
  Synthetic oligonucleotide
ccccctcccaggtgaccccaggtggaccctccccc

SEQ ID NO: 240 - DNA, 30 bp, Artificial Sequence
  Synthetic oligonucleotide
cccccacacctggggtcacctgggaccccc

SEQ ID NO: 241 - DNA, 30 bp, Artificial Sequence
  Synthetic oligonucleotide
ccccctcccaggtgaccccaggtgtccccc

SEQ ID NO: 242 - DNA, 25 bp, Artificial Sequence
  Synthetic oligonucleotide
ccccctggggtcacctgggaccccc

SEQ ID NO: 243 - DNA, 25 bp, Artificial Sequence
  Synthetic oligonucleotide
ccccctcccaggtgaccccaccccc

SEQ ID NO: 244-245 - skipped (2 entries)

SEQ ID NO: 246 - DNA, 40 bp, Artificial Sequence
  Synthetic oligonucleotide
gggggacaccagggtccacctggggtcacctgggaggggg

SEQ ID NO: 247 - DNA, 40 bp, Artificial Sequence
  Synthetic oligonucleotide
gggggtcccaggtgaccccaggtggaccctggtgtggggg

SEQ ID NO: 248 - DNA, 35 bp, Artificial Sequence
  Synthetic oligonucleotide
gggggagggtccacctggggtcacctgggaggggg

SEQ ID NO: 249 - DNA, 35 bp, Artificial Sequence
  Synthetic oligonucleotide
gggggtcccaggtgaccccaggtggaccctggggg

SEQ ID NO: 250 - DNA, 30 bp, Artificial Sequence
  Synthetic oligonucleotide
gggggacacctggggtcacctgggaggggg

SEQ ID NO: 251 - DNA, 30 bp, Artificial Sequence
  Synthetic oligonucleotide
gggggtcccaggtgaccccaggtgtggggg

SEQ ID NO: 252 - DNA, 25 bp, Artificial Sequence
  Synthetic oligonucleotide
gggggtggggtcacctgggaggggg

SEQ ID NO: 253 - DNA, 25 bp, Artificial Sequence
  Synthetic oligonucleotide
gggggtcccaggtgaccccaggggg

SEQ ID NO: 254-255 - skipped (2 entries)

SEQ ID NO: 256 - DNA, 45 bp, Artificial Sequence; identical to SEQ ID NO: 3
  Synthetic oligonucleotide
  misc_feature (6)(40): phosphorothioate linkages

SEQ ID NO: 257 - DNA, 45 bp, Artificial Sequence; identical to SEQ ID NO: 4
  Synthetic oligonucleotide
  misc_feature (6)(40): phosphorothioate linkages

SEQ ID NO: 258 - DNA, 45 bp, Artificial Sequence; identical to SEQ ID NO: 3
  Synthetic oligonucleotide
  misc_feature (17)(30): phosphorothioate linkages

SEQ ID NO: 259 - DNA, 45 bp, Artificial Sequence; identical to SEQ ID NO: 4
  Synthetic oligonucleotide
  misc_feature (16)(29): phosphorothioate linkages

SEQ ID NO: 260 - skipped

SEQ ID NO: 261 - DNA, 45 bp, Artificial Sequence
  Synthetic oligonucleotide
tagtgattatctagacatacatctagtacatgtctagtcagtata

SEQ ID NO: 262 - DNA, 45 bp, Artificial Sequence
  Synthetic oligonucleotide
tatactgactagacatgtactagatgtatgtctagataatcacta

SEQ ID NO: 263 - DNA, 50 bp, Artificial Sequence
  Synthetic oligonucleotide
atctagtgattatctagacatacatctagtacatgtctagtcagtatcta

SEQ ID NO: 264 - DNA, 50 bp, Artificial Sequence
  Synthetic oligonucleotide
tagatactgactagacatgtactagatgtatgtctagataatcactagat

SEQ ID NO: 265 - skipped

SEQ ID NO: 266 - DNA, 75 bp, Artificial Sequence; identical to SEQ ID NO: 21
  Synthetic oligonucleotide
  misc_feature (6)(70): phosphorothioate linkages

SEQ ID NO: 267 - DNA, 75 bp, Artificial Sequence; identical to SEQ ID NO: 22
  Synthetic oligonucleotide
  misc_feature (6)(70): phosphorothioate linkages

SEQ ID NO: 268 - DNA, 75 bp, Artificial Sequence; identical to SEQ ID NO: 21
  Synthetic oligonucleotide
  misc_feature (11)(65): phosphorothioate linkages

SEQ ID NO: 269 - DNA, 75 bp, Artificial Sequence; identical to SEQ ID NO: 22
  Synthetic oligonucleotide
  misc_feature (11)(65): phosphorothioate linkages

SEQ ID NO: 270 - DNA, 75 bp, Artificial Sequence; identical to SEQ ID NO: 21
  Synthetic oligonucleotide
  misc_feature (1)(75): phosphorothioate linkages

SEQ ID NO: 271 - DNA, 75 bp, Artificial Sequence; identical to SEQ ID NO: 22
  Synthetic oligonucleotide
  misc_feature (1)(75): phosphorothioate linkages

SEQ ID NO: 272 - DNA, 75 bp, Artificial Sequence; identical to SEQ ID NO: 21
  Synthetic oligonucleotide
  misc_feature (16)(61): phosphorothioate linkages

SEQ ID NO: 273 - DNA, 75 bp, Artificial Sequence; identical to SEQ ID NO: 21
  Synthetic oligonucleotide
  misc_feature (26)(51): phosphorothioate linkages

SEQ ID NO: 274 - DNA, 75 bp, Artificial Sequence
  Synthetic oligonucleotide
tttttagtcagtatctagtgattatctagacatacatctagtacatgtctagtcagtatctagtgattatttttt

SEQ ID NO: 275 - DNA, 75 bp, Artificial Sequence
  Synthetic oligonucleotide
tttttataatcactagatactgactagacatgtactagatgtatgtctagataatcactagatactgactttttt

SEQ ID NO: 276 - DNA, 75 bp, Artificial Sequence
  Synthetic oligonucleotide
gggggagtcagtatctagtgattatctagacatacatctagtacatgtctagtcagtatctagtgattatggggg

SEQ ID NO: 277 - DNA, 75 bp, Artificial Sequence
  Synthetic oligonucleotide
gggggataatcactagatactgactagacatgtactagatgtatgtctagataatcactagatactgactggggg

SEQ ID NO: 278 - DNA, 75 bp, Artificial Sequence
  Synthetic oligonucleotide
ccccctgggtgaaccagcaggtgggcaaagatgcagtcctagcaatgtaatcgtcaagctttatgccgttccccc

SEQ ID NO: 279 - DNA, 75 bp, Artificial Sequence
  Synthetic oligonucleotide
cccccaacggcataaagcttgacgattacattgctaggactgcatctttgcccacctgctggttcacccaccccc

SEQ ID NO: 280 - DNA, 75 bp, Artificial Sequence
  Synthetic oligonucleotide
uuuuuagtcagtatctagtgattatctagacatacatctagtacatgtctagtcagtatctagtgattatuuuuu

SEQ ID NO: 281 - DNA, 75 bp, Artificial Sequence
  Synthetic oligonucleotide
uuuuuataatcactagatactgactagacatgtactagatgtatgtctagataatcactagatactgactuuuuu

SEQ ID NO: 282 - DNA, 50 bp, Artificial Sequence
  Synthetic oligonucleotide
ccccuagtcagtatctagtgattatagtcagtatctagtgattatucccc

SEQ ID NO: 283 - DNA, 50 bp, Artificial Sequence
  Synthetic oligonucleotide
ccccuataatcactagatactgactataatcactagatactgactucccc